(19) United States
(12) Patent Application Publication
(10) Pub. No.: US 2026/0191817 A1
(43) Pub. Date: Jul. 9, 2026

(54) FIBROBLAST MODULATORS IN STROMA-RICH TUMORS

(71) Applicant: UNIVERSITEIT GENT, Gent (BE)

(72) Inventors: Christian STEVENS, Gent (BE); Bart ROMAN, Gent (BE); Christophe MANGODT, Gent (BE); Olivier DE WEVER, Gent (BE)

(21) Appl. No.: 18/868,562

(22) PCT Filed: May 31, 2023
(86) PCT No.: PCT/EP2023/064541

§ 371 Date: Nov. 22, 2024

(30) Foreign Application Priority Data
May 31, 2022 (EP) 22176556.3

(51) Int. Cl. A61K 31/352; A61K 31/12; C07C 49/84; C07D 311/30

(52) U.S. Cl. CPC A61K 31/352; A61K 31/12; C07C 49/84; C07D 311/30

(57) ABSTRACT

The present invention relates to the field of oncology and provides compounds, compositions, uses and methods for treating cancer, in particular stroma rich tumors, by selectively targeting cancer associated fibroblasts (CAFs). CAF targeting is one strategy for enhancing immunotherapy efficacy and may improve patient response rates. In addition, by this specificity in targets and associated pathways, the damaging of normal tissue is reduced.

### FIELD OF THE INVENTION

The present invention relates to the field of oncology and provides compounds, compositions, uses and methods for treating cancer, in particular stroma rich tumors, by selectively targeting cancer associated fibroblasts (CAFs). CAF targeting is one strategy for enhancing immunotherapy efficacy and may improve patient response rates. In addition, by this specificity in targets and associated pathways, the damaging of normal tissue is reduced.

### BACKGROUND TO THE INVENTION

Despite major advances in oncology, cancer remains a leading cause of death worldwide. In 2020, it accounted for nearly 10 million fatalities or nearly 17% of global mortality. The most common cancers in 2020 in terms of new cases were breast (2.26 million cases), lung (2.21 million cases) and colorectal cancer (1.93 million cases). In terms of mortality, lung (1.80 million deaths), colorectal (916 000 deaths) and liver cancer (830 000 deaths) are the leading cancer types. Breast cancer also remains a world-wide issue, with 685 000 deaths per annum.

A cancer is an abnormal growing mass composed of different cell types. The cancer cells form an ecosystem with their tumor microenvironment (TME) or stroma: other cell types and acellular matrix that can exert protumoral or antitumoral actions. The TME is a dynamic and complex organization of cellular and acellular components sculpted by chemical and physical interactions between cells of the tumor and of the neighboring structures. It encompasses elements of the vascular system, immune cells (e.g. T- and B-lymphocytes, natural killer (NK) cells and macrophages) and stromal cells (including pericytes and fibroblasts), as well as the acellular extracellular matrix (ECM). The ECM functions as a scaffold for cells in the TME: it provides structural support and modulates cell motility, proliferation and differentiation. It consists of a network of fibrous proteins, glycoproteins, proteoglycans and polysaccharides with diverse physical and biochemical properties, and is also rich in growth factors.

The TME is an essential and dynamic compartment that has a profound impact on all steps of tumor progression and on the therapeutic response. In a healthy state, the TME can help protect and reduce the invasiveness of tumor cells. However, cross-talks between tumor cells and components of the TME can result in an opposite reaction, which promotes tumor development, growth, drug resistance, immunosuppression and metastasis. A recent insight in the oncology field is, therefore, that cancer cell-centric therapeutic paradigms are generally not sufficient to eradicate malignancies. In order to improve cancer survival rates, there is an urgent unmet need for a range of therapeutic approaches that target the various cell types and processes that are the drivers of the protumoral events in the TME.

Among the stromal cells that populate the tumor microenvironment, cancer-associated fibroblasts (CAFs) are the most abundant and are critically involved in cancer progression. They comprise various fibroblast populations of distinct cellular origins that become activated during tumorigenesis (i.e. protumoral CAFs). CAFs regulate the biology of tumor cells and other stromal cells via cell-cell contacts, the release of numerous regulatory factors (e.g., transforming growth factor-β (TGF-β), vascular endothelial growth factor (VEGF), interleukin-6 (IL-6) and CXC-chemokine ligand (CXCL12)) and the synthesis and remodeling of the extracellular matrix. CAFs are drivers of protumoral events such as growth, drug resistance, angiogenesis, immunosuppression and metastasis.

The ECM is the noncellular component of the TME. Collagen is the most significant constituent of the ECM and its crosslinking is mediated primarily by lysyl oxidase (LOX), which is an amine oxidase that is frequently overexpressed in cancers. Expression of matrix metalloproteinases (MMPs) is also upregulated. These enzymes proteolytically degrade ECM components, which is important in the regulation of the ECM composition and enables the release of ECM-bound soluble factors, such as growth factors.

Besides being a supportive structure, the ECM is also a dynamic compartment responsible for the promotion of the malignant phenotype of cancer cells. Increased deposition of structural components (such as collagen, fibronectin and proteoglycans) and secretion of crosslinking enzymes (e.g. transglutaminases (TGMs) and lysyl oxidases (LOXs)) by CAFs cause the formation of a denser and stiffer TME and tumor in a process called desmoplasia. The physiological stiffness of natural tissues varies from 0.1 kPa in brain tissue to 40 kPa in the bone. In cancer patients, the stromal stiffness is around 5 kPa in the invasive regions, compared to 400 Pa in normal, non-invasive stroma. The higher stiffness of tumors is correlated with the amount of fibroblasts in the stroma and with advanced stages of carcinomas, therapy resistance, immunosuppression, a higher metastasis rate and poor patient prognosis. At present, fibroblast modulators as fitting therapeutic options in tumors with an important fibroblast presence (CAFs) are under investigation.

An essential requirement for any CAF modulator to be of practical therapeutic use in human cancer patients is pharmacological selectivity for CAFs with respect to fibroblasts and other related cell types implicated in normal physiology. Because of this required pharmacological selectivity, the development of oncology treatments that target CAFs has faced significant obstacles. Due to the poor specificity in targets and associated pathways, targeting CAFs without damaging normal tissue remains an unanswered challenge in the field.

It is the aim of the present invention to provide compounds for selectively targeting CAFs and thereby improving therapeutic outcome of cancer treatments.

### SUMMARY OF THE INVENTION

Accordingly, in a first aspect, the present invention provides a compound of Formula I or a salt, hydrate, or solvate thereof, for use in the treatment of invasive lobular breast carcinoma, tumors having a Tumor-Stroma Ratio (TSR) of >50%; and/or pancreatic ductal adenocarcinoma,

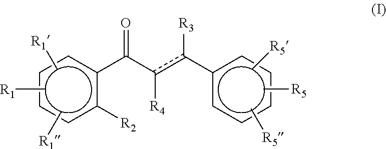

wherein
[symbol] represents a single or double bond;
each of said R₁, R₁′ and R₁″ is independently selected from the group consisting of —H, -halo, —CF₃, —OCF₃, and —OC₁₋₃alkyl;
R₂ is selected from the group consisting of —H, -halo, —CF₃, —OCF₃, and —OC₁₋₃alkyl;
R₃ is selected from the group consisting of —H, -halo, —C₁₋₃alkyl and —CF₃;
or R₂ and R₃ together with the C atoms to which they are attached form a 6-membered aromatic or non-aromatic heterocycle;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, and —OC₁₋₃alkyl, —C₁₋₃alkyl-OH and —OC₁₋₃alkyl-OH;
wherein at least one of said R₁, R₁′ and R₁″ is —OC₁₋₃alkyl or —CF₃;
wherein when R₂ and R₃ do not form a heterocycle then [symbol] represents a double bond.

In a specific embodiment, the compounds for use as defined herein are those wherein:
when R₂ and R₃ form a 6-membered aromatic or non-aromatic heterocycle, then said compound is represented by formula (II)

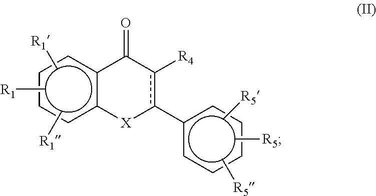

wherein X is selected from the group consisting of O, S, NH or N—C₁₋₃alkyl.

In another specific embodiment, the compounds, in particular of Formula I or II, for use as defined herein are those wherein:
[symbol] represents a single or double bond;
each of said R₁, R₁′ and R₁″ is independently selected from the group consisting of —H, —CF₃ and —OC₁₋₂alkyl;
R₂ is —H;
R₃ is selected from the group consisting of —H, and -Me;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆, and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is a 6-membered heterocycle having from 1 to 3 heteroatoms selected from O and N; wherein each of said Het₁ is optionally substituted with from 1 to 3 substituents selected from the group consisting of —C₁₋₂alkyl, and —C₁₋₂alkyl-OH;
wherein at least one of said R₁, R₁° and R₁″ is —OC₁₋₂alkyl;
wherein when R₂ and R₃ do not form a heterocycle then [symbol] represents a double bond.

In yet a further specific embodiment, the compounds for use as defined herein are those wherein: [symbol] represents a single or double bond;
each of said R₁, R₁′ and R₁″ is independently selected from the group consisting of —H, —CF₃ and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆, and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is selected from morpholinyl and piperazinyl; wherein each of said Het₁ is optionally substituted with from 1 to 3 substituents selected from the group consisting of —C₁₋₂alkyl, and —C₁₋₂alkyl-OH;
wherein at least one of said R₁, R₁′ and R₁″ is —OCH₃;
wherein when R₂ and R₃ do not form a heterocycle then [symbol] represents a double bond.

In a further embodiment, the present invention provides a compound, in particular of Formula I or II, for use as defined herein wherein:
[symbol] represents a single or double bond;
each of said R₁, R₁′ and R₁″ is independently selected from the group consisting of —H, and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, and -halo;
wherein at least one of said R₁, R₁′ and R₁″ is —OCH₃;
wherein when R₂ and R₃ do not form a heterocycle then [symbol] represents a double bond.

In yet a further embodiment, the present invention provides a compound, in particular of Formula I or II, for use as defined herein wherein:
each of said R₁, R₁′ and R₁″ is independently selected from the group consisting of —H, and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —F and —Cl;
wherein at least one of said R₁, R₁′ and R₁″ is —OCH₃;
wherein when R₂ and R₃ do not form a heterocycle then [symbol] represents a double bond.

The compounds for use as defined herein may in a specific embodiment be represented by any one of formula (Ia), (IIa) or (IIb)

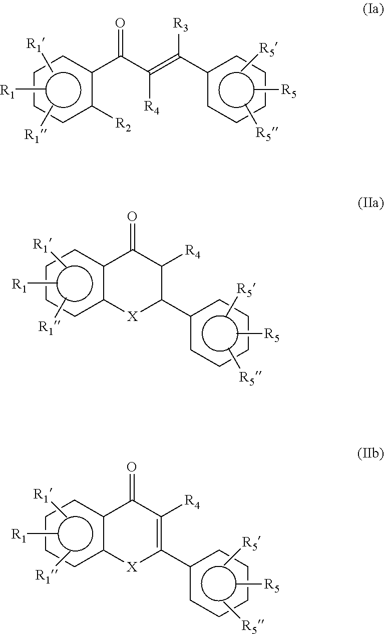

The present invention also provides a compound for use as defined herein, wherein said compound or a salt, hydrate, or solvate thereof is selected from the list comprising:

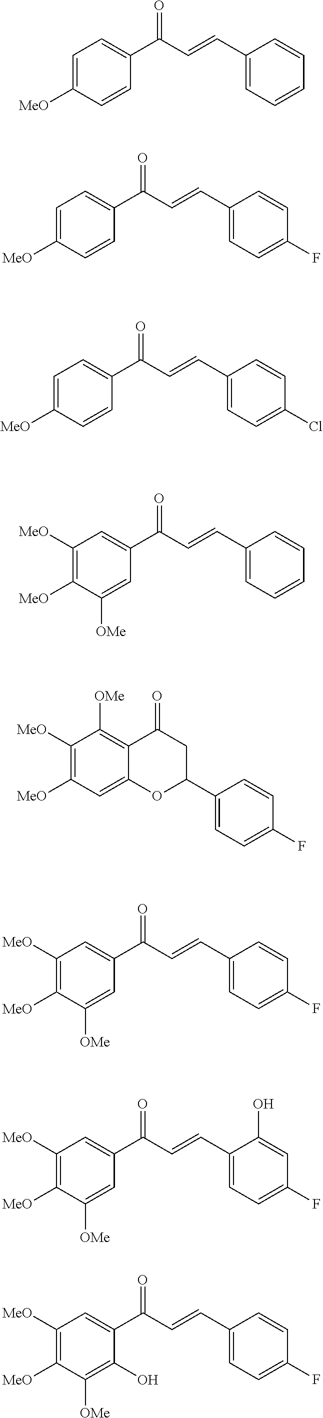

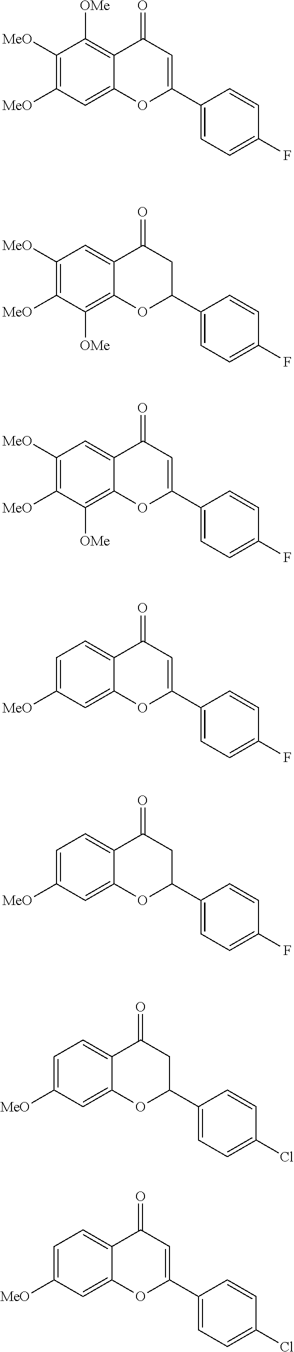

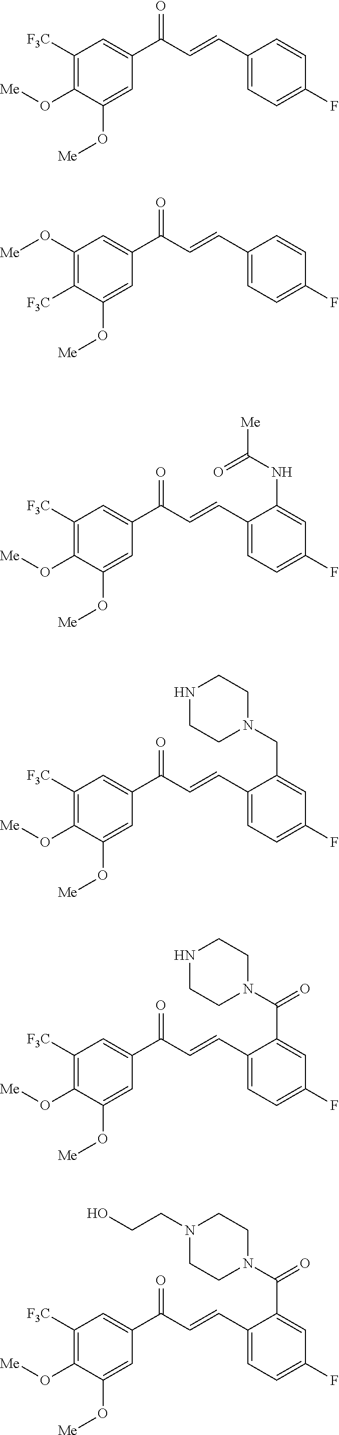 [SMILES: COc1cc(C(=O)/C=C/c2ccc(F)cc2)cc(C(F)(F)F)c1OC ; COc1cc(C(=O)/C=C/c2ccc(F)cc2)cc(OC)c1C(F)(F)F ; COc1cc(C(=O)/C=C/c2ccc(F)cc2NC(C)=O)cc(C(F)(F)F)c1OC ; COc1cc(C(=O)/C=C/c2ccc(F)cc2CN2CCNCC2)cc(C(F)(F)F)c1OC ; COc1cc(C(=O)/C=C/c2ccc(F)cc2C(=O)N2CCNCC2)cc(C(F)(F)F)c1OC ; COc1cc(C(=O)/C=C/c2ccc(F)cc2C(=O)N2CCN(CCO)CC2)cc(C(F)(F)F)c1OC]

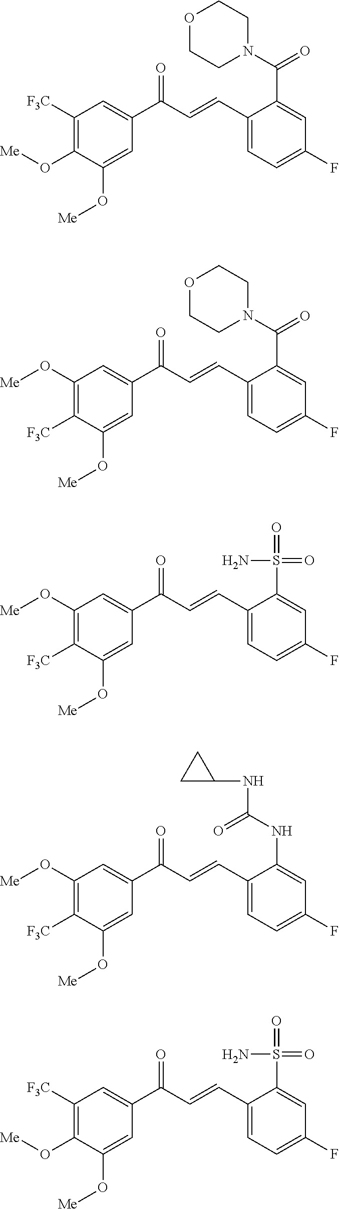 [SMILES: COc1cc(C(=O)/C=C/c2ccc(F)cc2C(=O)N2CCOCC2)cc(C(F)(F)F)c1OC ; COc1cc(C(=O)/C=C/c2ccc(F)cc2C(=O)N2CCOCC2)cc(OC)c1C(F)(F)F ; COc1cc(C(=O)/C=C/c2ccc(F)cc2S(N)(=O)=O)cc(OC)c1C(F)(F)F ; COc1cc(C(=O)/C=C/c2ccc(F)cc2NC(=O)NC2CC2)cc(OC)c1C(F)(F)F ; COc1cc(C(=O)/C=C/c2ccc(F)cc2S(N)(=O)=O)cc(C(F)(F)F)c1OC]

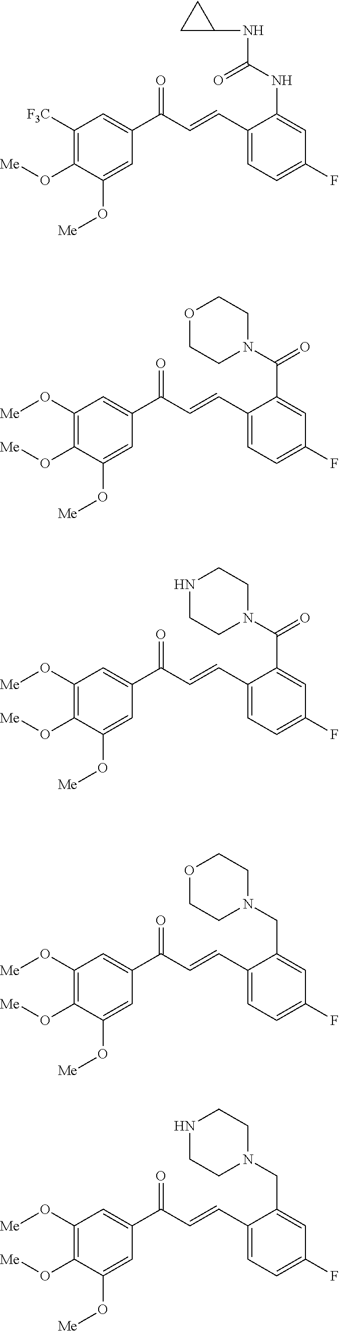 [SMILES: COc1cc(C(=O)/C=C/c2ccc(F)cc2NC(=O)NC2CC2)cc(C(F)(F)F)c1OC ; COc1cc(C(=O)/C=C/c2ccc(F)cc2C(=O)N2CCOCC2)cc(OC)c1OC ; COc1cc(C(=O)/C=C/c2ccc(F)cc2C(=O)N2CCNCC2)cc(OC)c1OC ; COc1cc(C(=O)/C=C/c2ccc(F)cc2CN2CCOCC2)cc(OC)c1OC ; COc1cc(C(=O)/C=C/c2ccc(F)cc2CN2CCNCC2)cc(OC)c1OC]

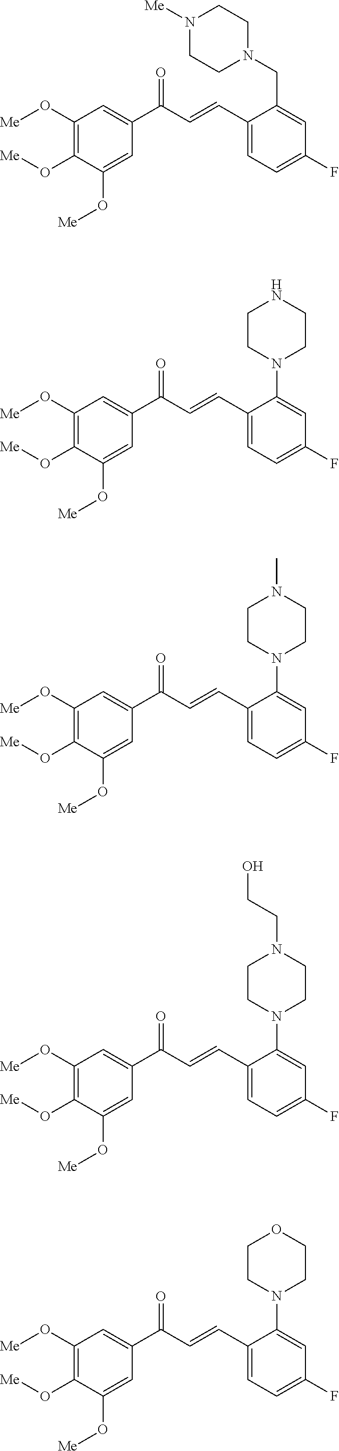 [SMILES: COc1cc(C(=O)/C=C/c2ccc(F)cc2CN2CCN(C)CC2)cc(OC)c1OC ; COc1cc(C(=O)/C=C/c2ccc(F)cc2N2CCNCC2)cc(OC)c1OC ; COc1cc(C(=O)/C=C/c2ccc(F)cc2N2CCN(C)CC2)cc(OC)c1OC ; COc1cc(C(=O)/C=C/c2ccc(F)cc2N2CCN(CCO)CC2)cc(OC)c1OC ; COc1cc(C(=O)/C=C/c2ccc(F)cc2N2CCOCC2)cc(OC)c1OC]

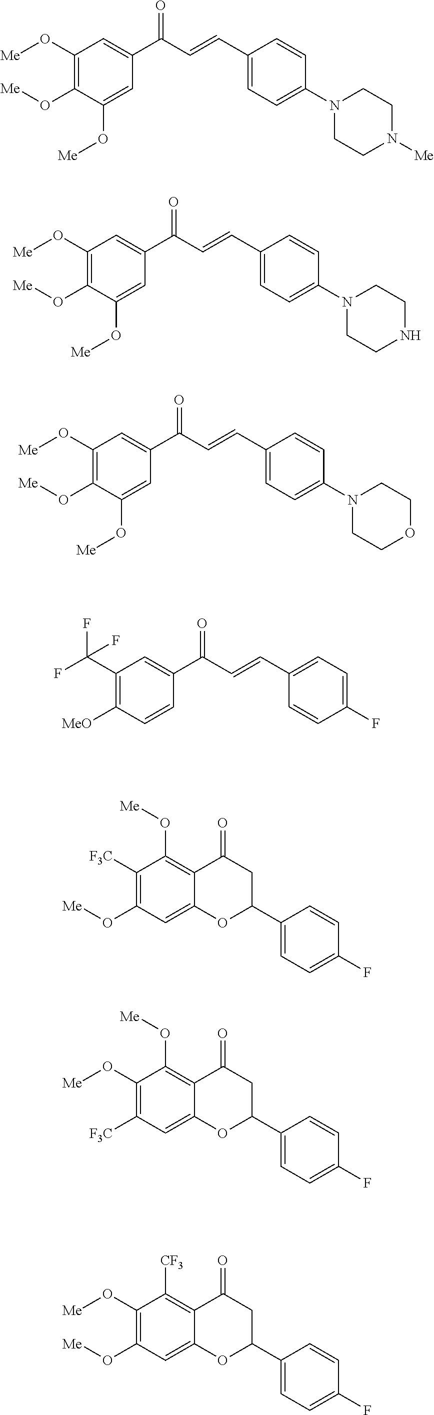

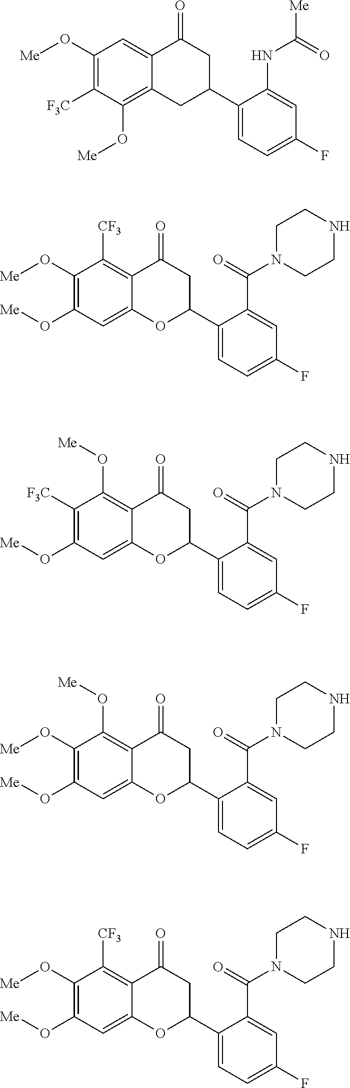 [SMILES: COc1cc2c(c(OC)c1C(F)(F)F)CC(c1ccc(F)cc1NC(C)=O)CC2=O ; COc1cc2c(c(C(F)(F)F)c1OC)C(=O)CC(c1ccc(F)cc1C(=O)N1CCNCC1)O2 ; COc1cc2c(c(OC)c1C(F)(F)F)C(=O)CC(c1ccc(F)cc1C(=O)N1CCNCC1)O2 ; COc1cc2c(c(OC)c1OC)C(=O)CC(c1ccc(F)cc1C(=O)N1CCNCC1)O2 ; COc1cc2c(c(C(F)(F)F)c1OC)C(=O)CC(c1ccc(F)cc1C(=O)N1CCNCC1)O2]

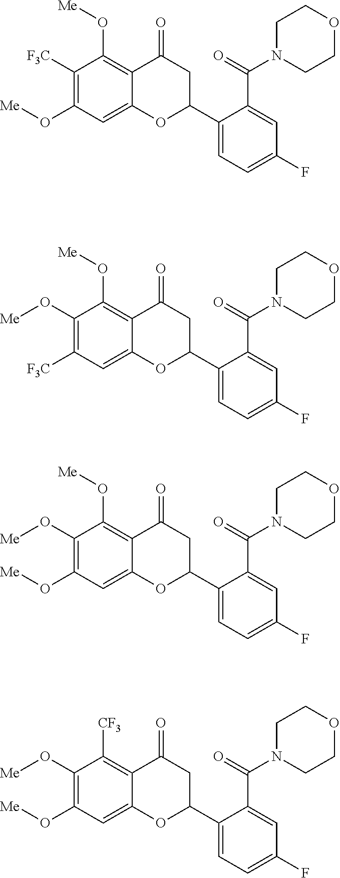 [SMILES: COc1cc2c(c(OC)c1C(F)(F)F)C(=O)CC(c1ccc(F)cc1C(=O)N1CCOCC1)O2 ; COc1c(C(F)(F)F)cc2c(c1OC)C(=O)CC(c1ccc(F)cc1C(=O)N1CCOCC1)O2 ; COc1cc2c(c(OC)c1OC)C(=O)CC(c1ccc(F)cc1C(=O)N1CCOCC1)O2 ; COc1cc2c(c(C(F)(F)F)c1OC)C(=O)CC(c1ccc(F)cc1C(=O)N1CCOCC1)O2]

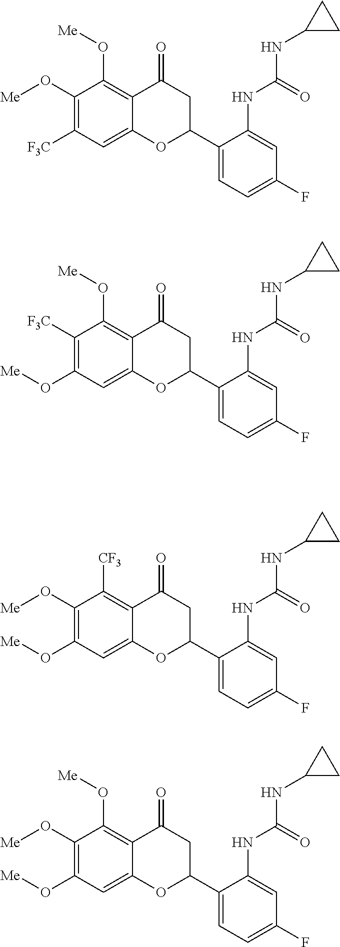

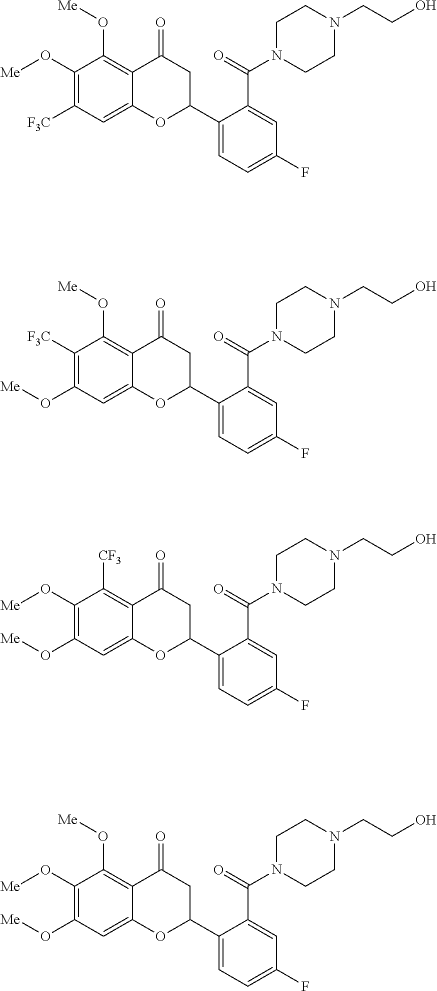 [SMILES: COc1c(C(F)(F)F)cc2c(c1OC)C(=O)CC(c1ccc(F)cc1C(=O)N1CCN(CCO)CC1)O2 ; COc1cc2c(c(OC)c1C(F)(F)F)C(=O)CC(c1ccc(F)cc1C(=O)N1CCN(CCO)CC1)O2 ; COc1cc2c(c(C(F)(F)F)c1OC)C(=O)CC(c1ccc(F)cc1C(=O)N1CCN(CCO)CC1)O2 ; COc1cc2c(c(OC)c1OC)C(=O)CC(c1ccc(F)cc1C(=O)N1CCN(CCO)CC1)O2]

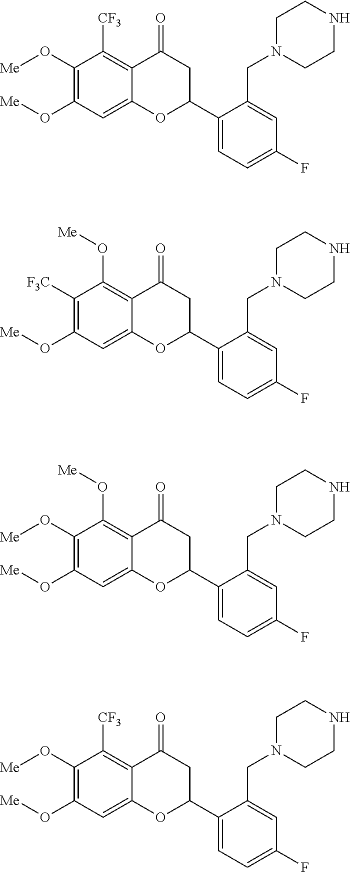 [SMILES: COc1cc2c(c(C(F)(F)F)c1OC)C(=O)CC(c1ccc(F)cc1CN1CCNCC1)O2 ; COc1cc2c(c(OC)c1C(F)(F)F)C(=O)CC(c1ccc(F)cc1CN1CCNCC1)O2 ; COc1cc2c(c(OC)c1OC)C(=O)CC(c1ccc(F)cc1CN1CCNCC1)O2 ; COc1cc2c(c(C(F)(F)F)c1OC)C(=O)CC(c1ccc(F)cc1CN1CCNCC1)O2]

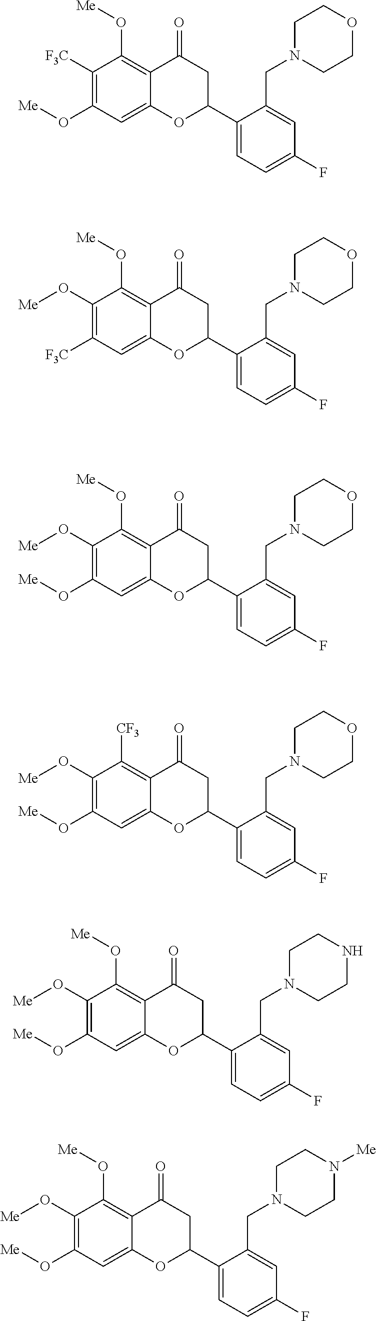 [SMILES: COc1cc2c(c(OC)c1C(F)(F)F)C(=O)CC(c1ccc(F)cc1CN1CCOCC1)O2 ; COc1c(C(F)(F)F)cc2c(c1OC)C(=O)CC(c1ccc(F)cc1CN1CCOCC1)O2 ; COc1cc2c(c(OC)c1OC)C(=O)CC(c1ccc(F)cc1CN1CCOCC1)O2 ; COc1cc2c(c(C(F)(F)F)c1OC)C(=O)CC(c1ccc(F)cc1CN1CCOCC1)O2 ; COc1cc2c(c(OC)c1OC)C(=O)CC(c1ccc(F)cc1CN1CCNCC1)O2 ; COc1cc2c(c(OC)c1OC)C(=O)CC(c1ccc(F)cc1CN1CCN(C)CC1)O2]

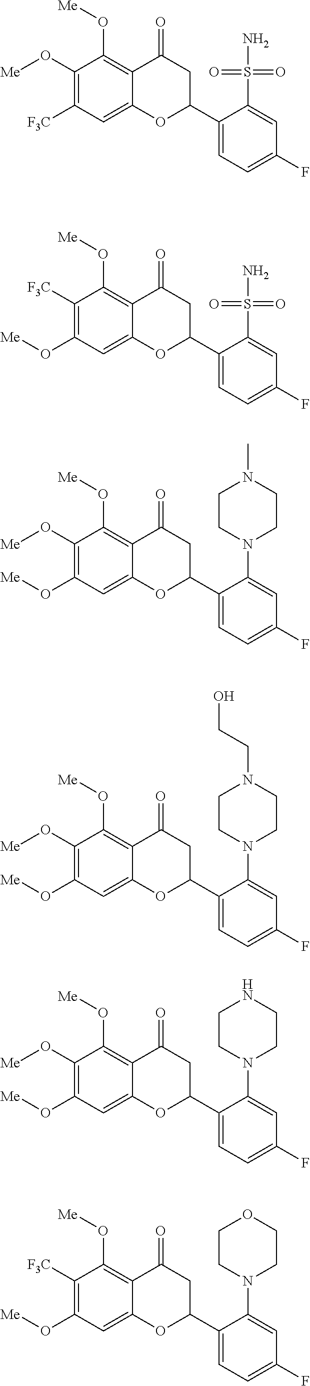

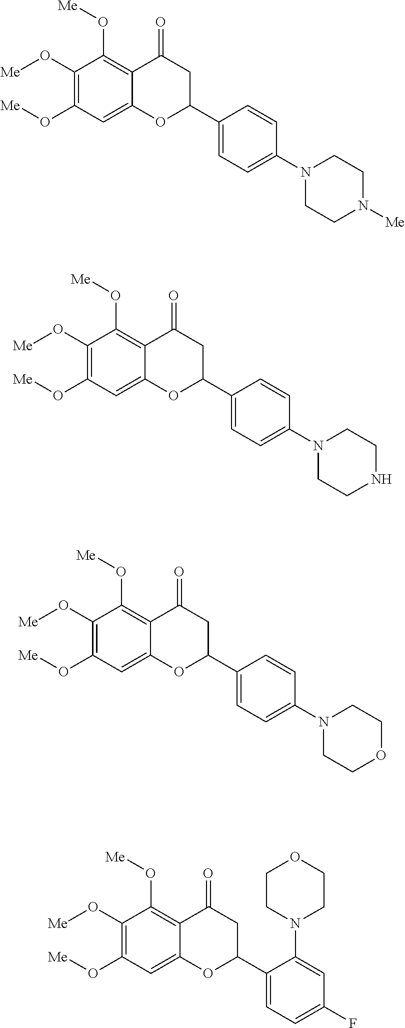 [SMILES: COc1cc2c(c(OC)c1OC)C(=O)CC(c1ccc(N3CCN(C)CC3)cc1)O2 ; COc1cc2c(c(OC)c1OC)C(=O)CC(c1ccc(N3CCNCC3)cc1)O2 ; COc1cc2c(c(OC)c1OC)C(=O)CC(c1ccc(N3CCOCC3)cc1)O2 ; COc1cc2c(c(OC)c1OC)C(=O)CC(c1ccc(F)cc1N1CCOCC1)O2]

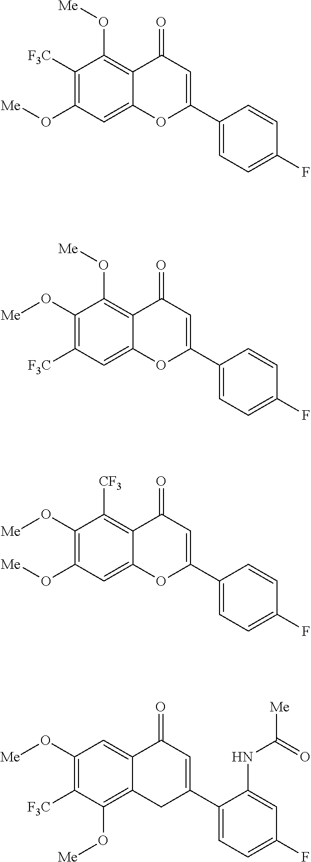 [SMILES: COc1cc2oc(-c3ccc(F)cc3)cc(=O)c2c(OC)c1C(F)(F)F ; COc1c(C(F)(F)F)cc2oc(-c3ccc(F)cc3)cc(=O)c2c1OC ; COc1cc2oc(-c3ccc(F)cc3)cc(=O)c2c(C(F)(F)F)c1OC ; COc1cc2c(c(OC)c1C(F)(F)F)CC(c1ccc(F)cc1NC(C)=O)=CC2=O]

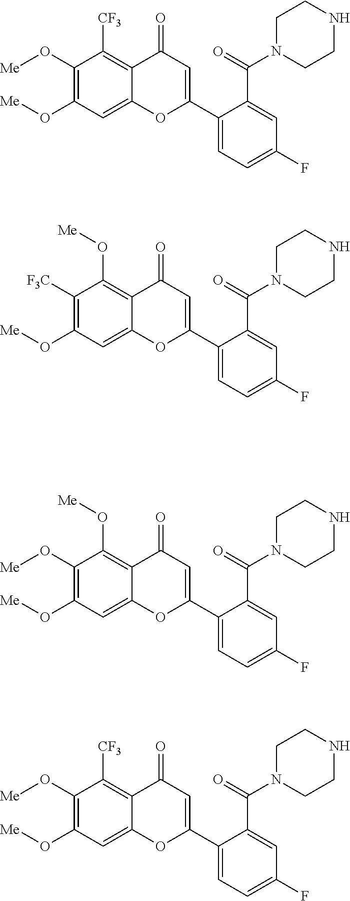 [SMILES: COc1cc2oc(-c3ccc(F)cc3C(=O)N3CCNCC3)cc(=O)c2c(C(F)(F)F)c1OC ; COc1cc2oc(-c3ccc(F)cc3C(=O)N3CCNCC3)cc(=O)c2c(OC)c1C(F)(F)F ; COc1cc2oc(-c3ccc(F)cc3C(=O)N3CCNCC3)cc(=O)c2c(OC)c1OC ; COc1cc2oc(-c3ccc(F)cc3C(=O)N3CCNCC3)cc(=O)c2c(C(F)(F)F)c1OC]

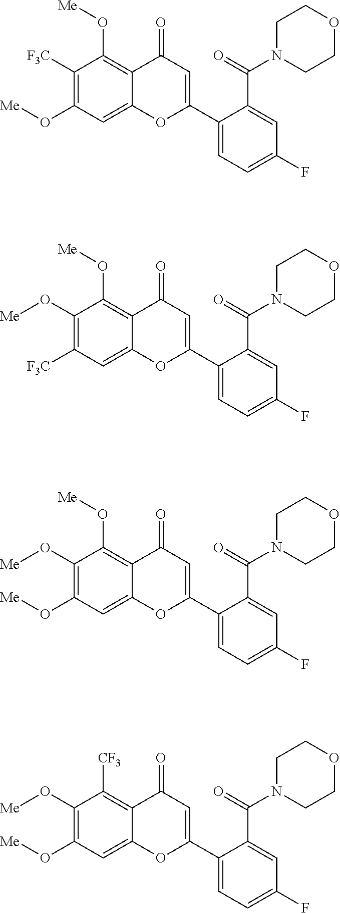 [SMILES: COc1cc2oc(-c3ccc(F)cc3C(=O)N3CCOCC3)cc(=O)c2c(OC)c1C(F)(F)F ; COc1c(C(F)(F)F)cc2oc(-c3ccc(F)cc3C(=O)N3CCOCC3)cc(=O)c2c1OC ; COc1cc2oc(-c3ccc(F)cc3C(=O)N3CCOCC3)cc(=O)c2c(OC)c1OC ; COc1cc2oc(-c3ccc(F)cc3C(=O)N3CCOCC3)cc(=O)c2c(C(F)(F)F)c1OC]

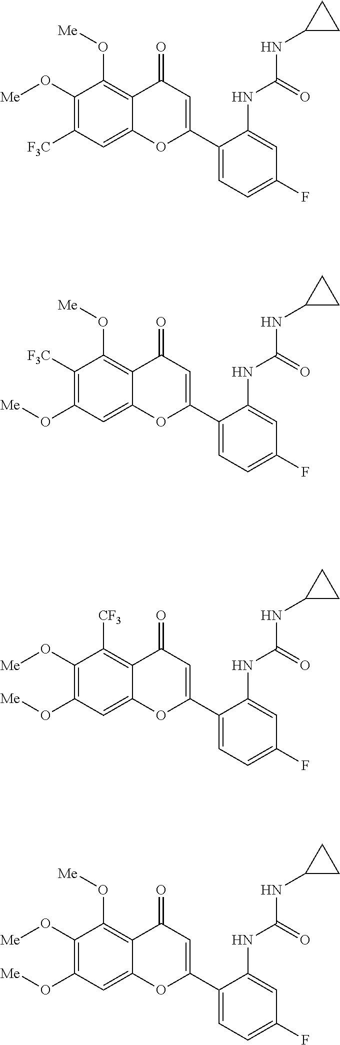 [SMILES: COc1c(C(F)(F)F)cc2oc(-c3ccc(F)cc3NC(=O)NC3CC3)cc(=O)c2c1OC ; COc1cc2oc(-c3ccc(F)cc3NC(=O)NC3CC3)cc(=O)c2c(OC)c1C(F)(F)F ; COc1cc2oc(-c3ccc(F)cc3NC(=O)NC3CC3)cc(=O)c2c(C(F)(F)F)c1OC ; COc1cc2oc(-c3ccc(F)cc3NC(=O)NC3CC3)cc(=O)c2c(OC)c1OC]

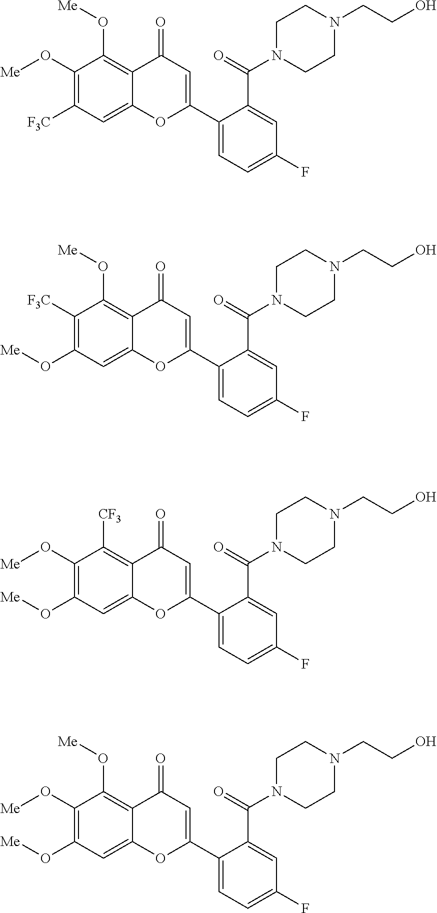 [SMILES: COc1c(C(F)(F)F)cc2oc(-c3ccc(F)cc3C(=O)N3CCN(CCO)CC3)cc(=O)c2c1OC ; COc1cc2oc(-c3ccc(F)cc3C(=O)N3CCN(CCO)CC3)cc(=O)c2c(OC)c1C(F)(F)F ; COc1cc2oc(-c3ccc(F)cc3C(=O)N3CCN(CCO)CC3)cc(=O)c2c(C(F)(F)F)c1OC ; COc1cc2oc(-c3ccc(F)cc3C(=O)N3CCN(CCO)CC3)cc(=O)c2c(OC)c1OC]

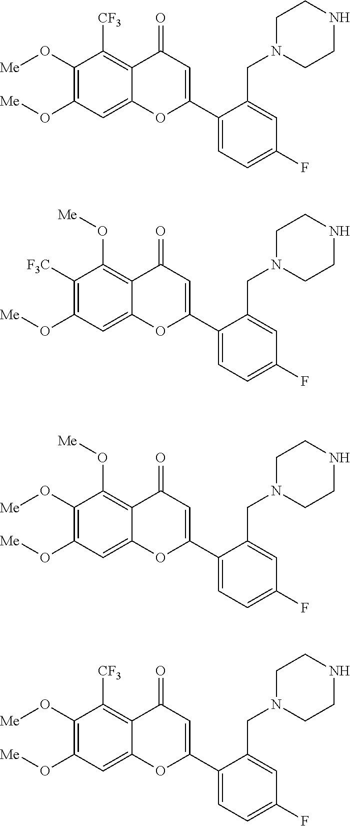

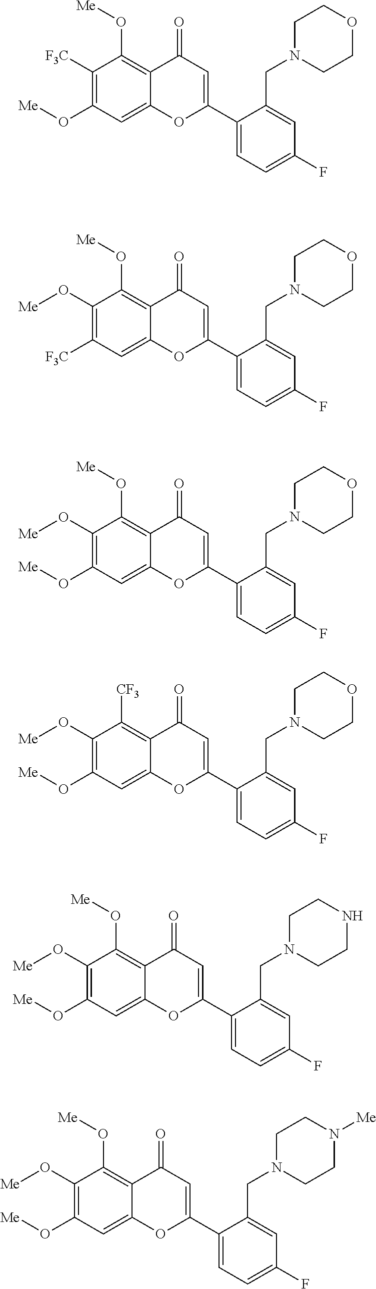

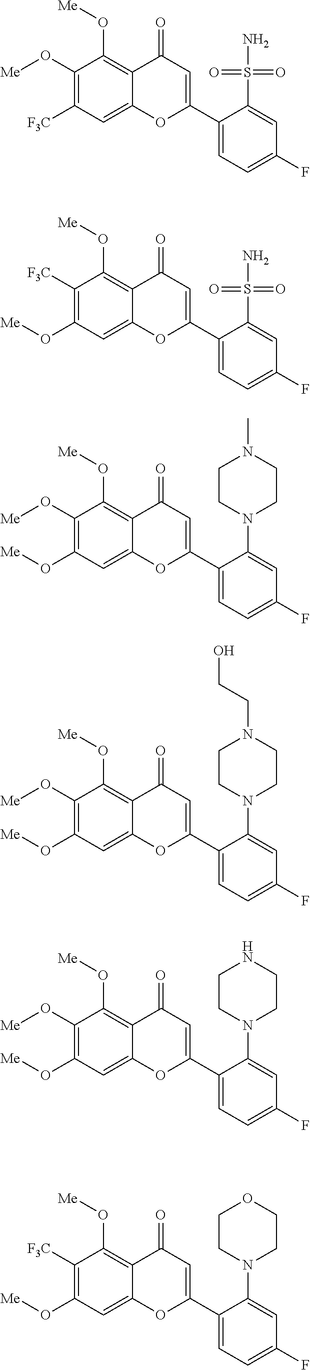

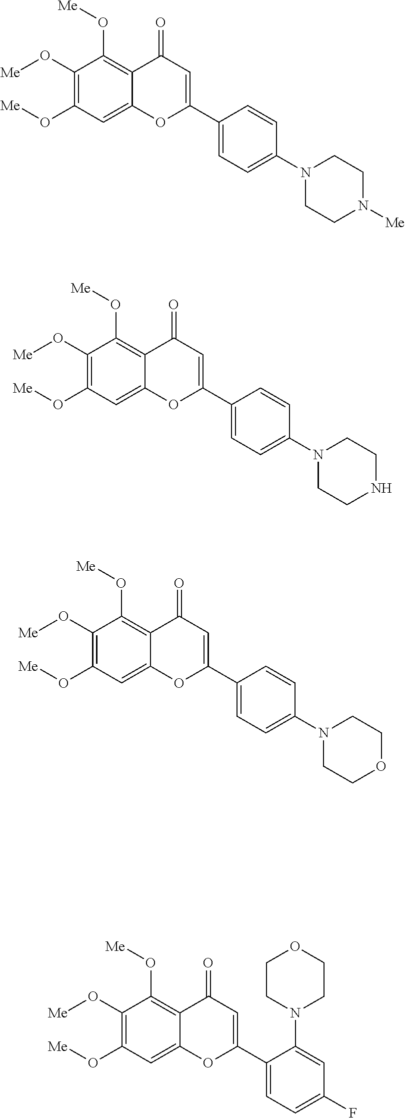 [SMILES: COc1cc2oc(-c3ccc(N4CCN(C)CC4)cc3)cc(=O)c2c(OC)c1OC ; COc1cc2oc(-c3ccc(N4CCNCC4)cc3)cc(=O)c2c(OC)c1OC ; COc1cc2oc(-c3ccc(N4CCOCC4)cc3)cc(=O)c2c(OC)c1OC ; COc1cc2oc(-c3ccc(F)cc3N3CCOCC3)cc(=O)c2c(OC)c1OC]

In another aspect, the present invention provides a compound or a salt, hydrate, or solvate thereof, selected from the list comprising:

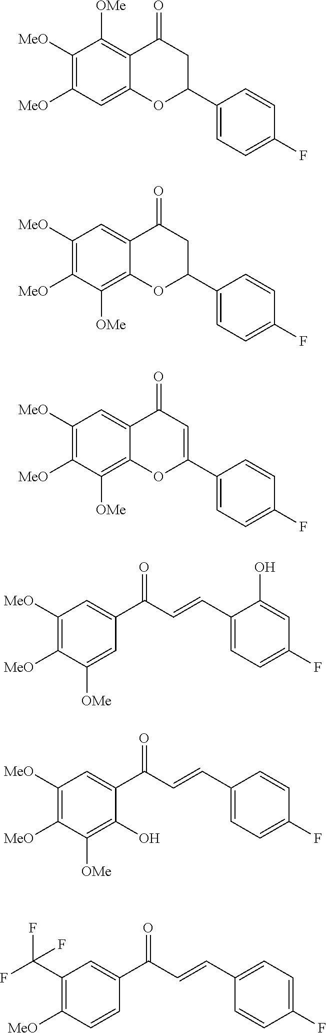

The present invention also provides a pharmaceutical composition comprising a compound as defined herein and a pharmaceutically acceptable excipient.

In a further embodiment, the present invention provides a compound or a composition as defined herein; for use in human or veterinary medicine; in particular for use in the treatment of cancer; more particular for use in preventing, treating or reducing tumor-associated desmoplasia in a subject having cancer, even more in particular for use in the treatment of invasive lobular breast carcinoma, tumors having a Tumor-Stroma Ratio (TSR) of >50%; and/or pancreatic ductal adenocarcinoma.

The present invention also provides a method for the prevention, reduction and/or treatment of tumor-associated desmoplasia in a subject having cancer, in particular for preventing or treating invasive lobular breast carcinoma, tumors having a Tumor-Stroma Ratio (TSR) of >50%; and/or pancreatic ductal adenocarcinoma; said method comprising administering to a subject in need thereof a therapeutically effective amount of a compound or a pharmaceutical composition as defined herein.

In yet a further aspect, the present invention provides a compound for use as an anti-invasive compound of tumor cells; said compound being represented by any one of formula (IIa) or (IIb)

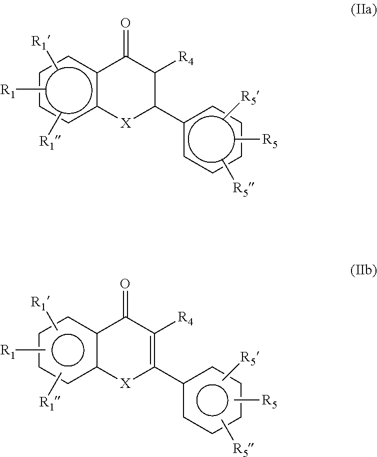

wherein
each of said R₁, R₁′ and R₁″ is independently selected from the group consisting of —H, -halo, —CF₃, —OCF₃, and —OC₁₋₃alkyl;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, —OC₁₋₃alkyl, —C₁₋₃alkyl-OH and —OC₁₋₃alkyl-OH;
wherein at least one of said R₁, R₁′ and R₁″ is —OC₁₋₃alkyl or —CF₃.

### DETAILED DESCRIPTION OF THE DRAWINGS

With specific reference to the figures, it is to be noted that the particulars shown are by way of example and for purposes of illustrative discussion of the different embodiments of the present invention. The description taken with the figures make it apparent to those skilled in the art how the several forms of the invention may be embodied in practice.

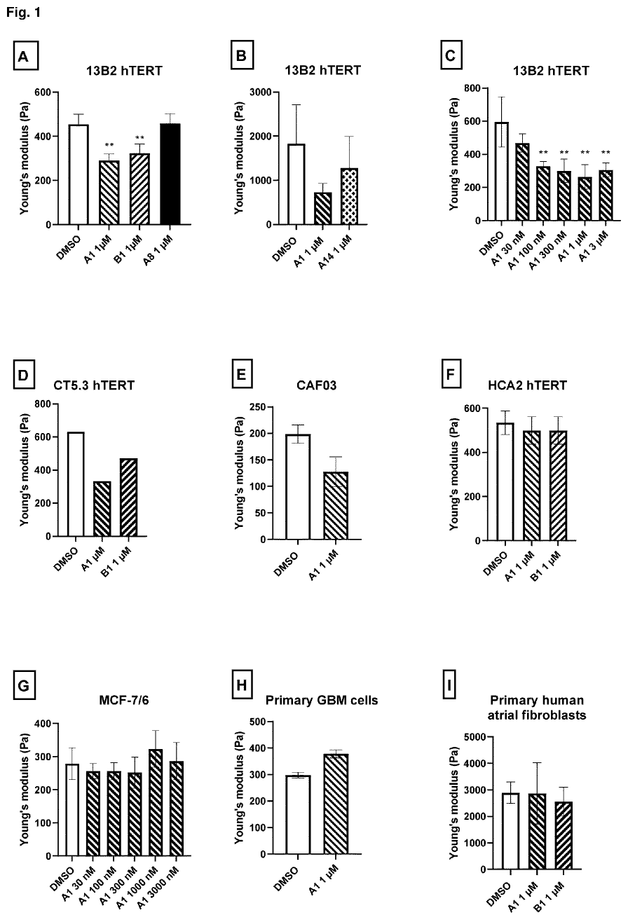

FIG. 1. Stiffness measurements of spheroids treated with A1, A8, A14 or B1 for 48 h during spheroid formation. A-C: 13B2 hTERT breast CAFs, D: CT5.3 hTERT colon CAFs, E: CAF03 glioblastoma CAFs, F: HCA2 hTERT dermal fibroblasts, G::MCF-7/6 breast cancer cells, H: primary glioblastoma (GBM) cells, I: primary human atrial fibroblasts. Data are expressed as mean±SD from three technical replicates. *P<0.05, **P<0.01.

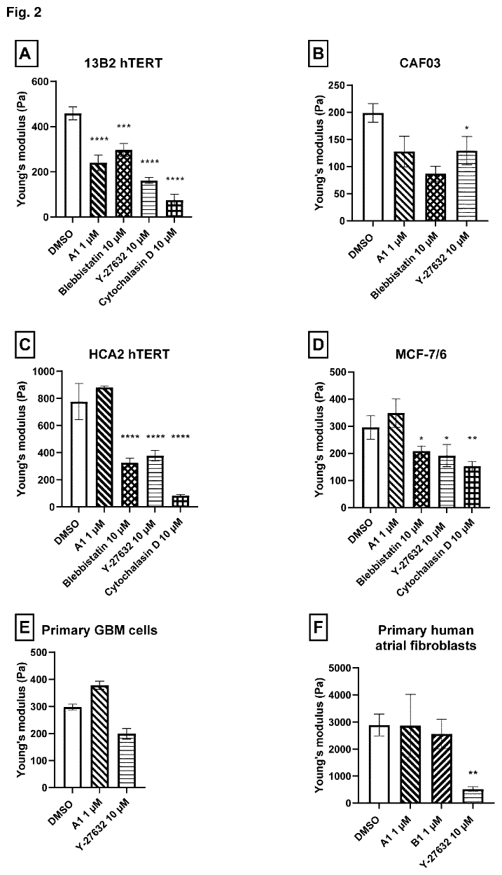

FIG. 2. Stiffness measurements of spheroids treated with 1 μM A1, 1 μM B1, 10 μM blebbistatin, 10 μM cytochalasin D or 10 μM Y-27632 for 48 h during spheroid formation. A: 13B2 hTERT breast CAFs, B: CAF03 glioblastoma CAFs, C: HCA2 hTERT dermal fibroblasts, D::MCF-7/6 breast cancer cells, E: primary glioblastoma cells, F: primary human atrial fibroblasts. Data are expressed as mean±SD from three technical replicates. *P<0.05, **P<0.01, ***P<0.001, ****P<0.0001.

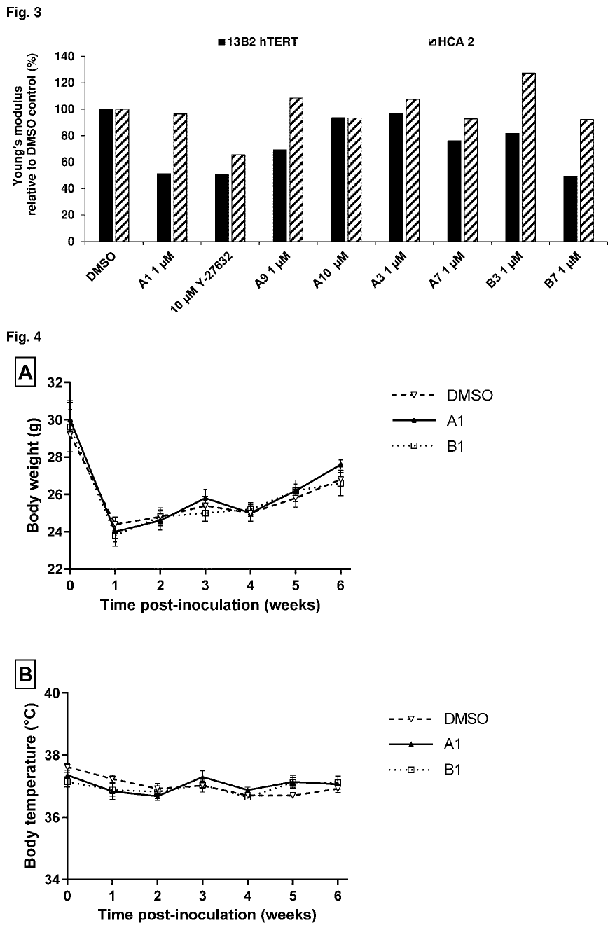

FIG. 3. Further selectivity data for compounds of the invention: comparison of stiffness measurements of spheroids of 13B2 hTERT breast CAFs and HCA2 hTERT dermal fibroblasts, treated with 1 μM A1, 1 μM A9, 1 μM A10, 1 μM A3, 1 μM A7, 1 μM B3, 1 μM B7 or 10 μM Y-27632 for 48 h during spheroid formation. Data are expressed as mean±SD from three technical replicates.

FIG. 4. 4T1 Animal model: tolerability of treatment with A1 or B1 versus DMSO control. A: Body weight; B: body temperature.

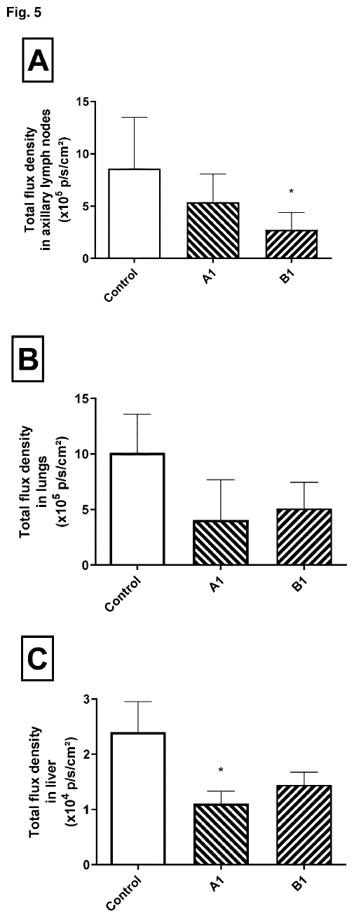

FIG. 5. 4T1 Animal model: reduction of metastatic burden on treatment with A1 or B1, compared to DMSO. A: Axillary lymph nodes ex vivo bioluminescence; B: Lung ex vivo bioluminescence; C: Liver ex vivo bioluminescence.

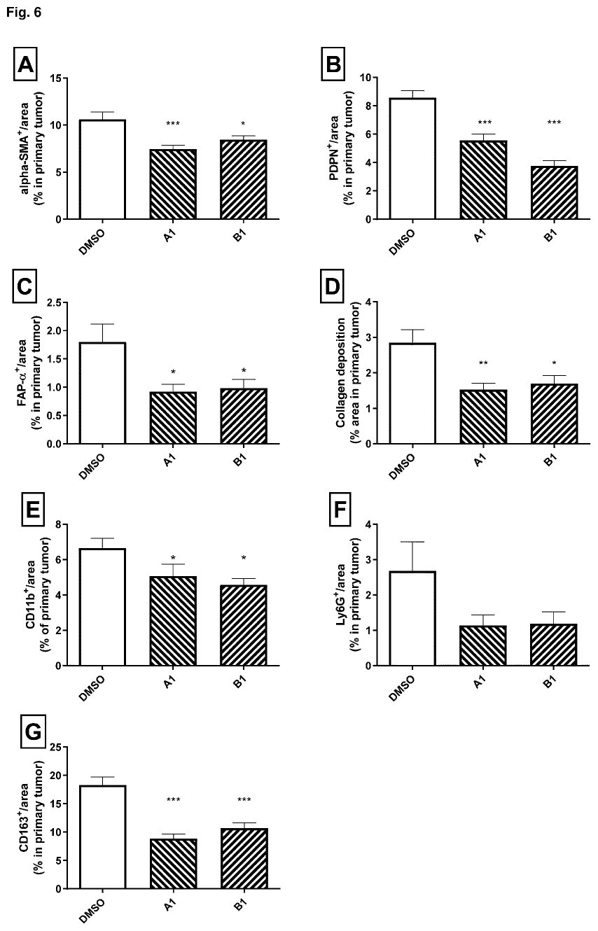

FIG. 6. 4T1 Animal model: immunohistological stainings of primary tumor material on treatment with A1 or B1, compared to control animals. A: α-SMA; B: PDPN; C: FAP-α; D: Masson's trichrome staining (collagen deposition); E: CD11b; F: Ly6G; G: CD163. *P<0.05, **P<0.01, ***P<0.001.

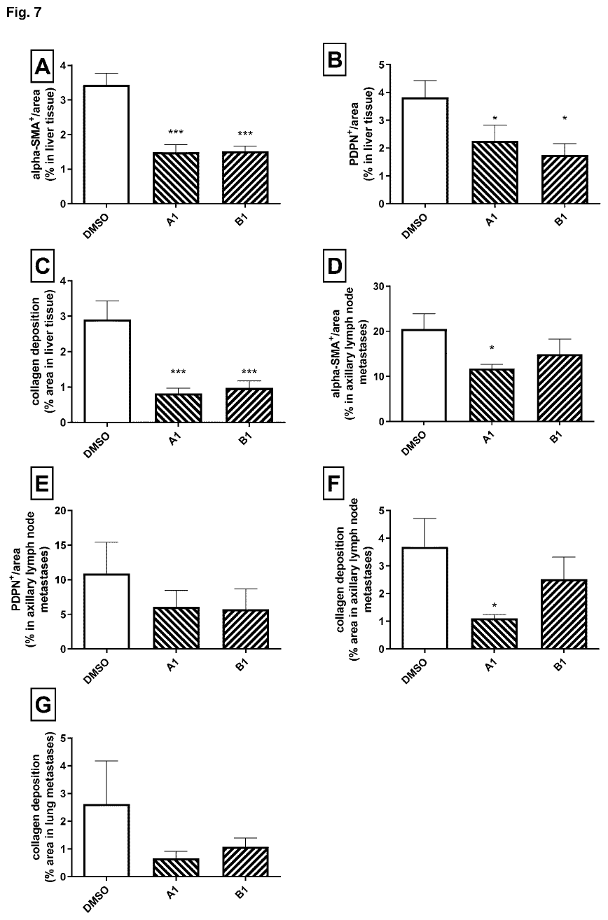

FIG. 7. 4T1 Animal model: immunohistochemical staining of target organs for metastasis on treatment with A1 or B1, compared to control animals. A: α-SMA, liver; B: PDPN, liver; C: Masson's trichrome staining (collagen deposition), liver; D: α-SMA, axillary lymph nodes; E: PDPN, axillary lymph nodes; F: Masson's trichrome staining, axillary lymph nodes; G: Masson's trichrome staining, lung. *P<0.05. **P<0.01. ***P<0.001.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. As used in the specification and the claims, the singular forms “a”, “an”, and “the” include plural referents unless the context clearly dictates otherwise. By way of example, “a compound” means one compound or more than one compound. Throughout the description and claims of this specification the word “comprise” and other forms of the word, such as “comprising” and “comprises,” means including but not limited to, and is not intended to exclude, for example, other additives, components, integers, or steps. As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus 10% of the particular value or term. The terms described above and others used in the specification are well understood to those in the art. All references, and teachings specifically referred to, cited in the present specification are hereby incorporated by reference in their entirety.

The present invention provides CAF (cancer-associated fibroblast) modulating compounds and more particular their use in treating cancer, more specific their use in reducing stiffness of the tumor microenvironment (TME) (also referred to as desmoplasia).

Fibroblasts represent one of the most abundant cell types in the stroma, and are often defined by a combination of their morphology, tissue position, and lack of lineage markers for epithelial cells, endothelial cells, and leukocytes.

Like fibroblasts in the stroma, cancer-associated fibroblasts (CAFs) are the main component in the tumor microenvironment (TME). Many of the features defined for normal fibroblasts also apply to CAFs. The expression “cancer-associated fibroblast” is used as an umbrella term to describe a complex group of mesenchymal-like cells. In Han et al (Han, Liu, and Yin 2020) it is even considered as the generic label for “all the fibroblasts found within and surrounding tumor tissues”. Next to their elongated morphology, the simplest characterization is a list of negative markers. CAFs are cells negative for epithelial, endothelial, and leukocyte markers and all mutations found within cancer cells. The latter point is important because it excludes cancer cells that have undergone an epithelial to mesenchymal transition (EMT). To make the interpretation narrower, lineage exclusion is typically combined with the presence of a mesenchymal marker, often vimentin. It has become clear that there are subtypes of CAFs with each their specific markers. In practice, any mesenchymal cell derived from a tumor that fulfills the criteria described above is considered a CAF.

The compounds of the invention are unique in that they are selective modulators of CAFs, thereby not or less targeting fibroblasts and other related cell types implicated in normal physiology. As such, the compounds of the invention can be used to target CAFs without damaging normal tissue. It was shown that the compounds provided herein functionally inhibit CAFs at nanomolar concentrations which is highly desired for active pharmaceutical ingredients.

It is well established that an increased tumor and ECM stiffness is associated with a negative prognosis as it promotes tumor growth, progression, therapy resistance, immunosuppression and metastasis. Stiffness is chiefly regulated by the action of the CAFs in the TME. Stiffness modulation is, therefore, a fitting read-out of activity in a screen for CAF modulators. To assess stiffness modulation, the stiffness of spheroids of CAF cells treated with test compounds was interrogated using atomic force microscopy (AFM) indentation and compared with untreated cells. AFM measurement of the stiffness (elasticity) of cells and spheroids is a technique well-known to those skilled in the art. AFM measures the force required to create a given deflection, this read-out is also known as the Young's modulus. AFM is widely used in biology and cancer research: altered physicochemical properties of live cells serve as a read-out of altered complex physiological processes.

Selectivity for CAFs with respect to normal fibroblasts and other cell types was assessed by performing an identical experiment with normal fibroblasts and other cell types, where compounds were considered selective for CAFs if they only modulate CAF stiffness.

The tumor stroma can limit access of therapeutic agents to their target tissues in three ways: fibrosis, high interstitial pressure, and degradation of drugs by stromal enzymes. The buildup of a rigid ECM (fibrosis) around and throughout a tumor creates a physical barrier that reduces diffusion of therapeutic agents to cancer cells. Similarly, the tumor stroma can limit access and actions of immunocytes in two ways: fibrosis, creating a physical barrier that reduces infiltration of immunocytes, and chemical and physical cues that favor protumoral, immunosuppressive immunocyte populations and induces polarization or differentiation of immunocytes to immunosuppressive phenotypes.

The present invention provides compounds as defined herein for use in treating cancer by reducing said rigid ECM and immunosuppressive cues/polarization/differentiation, in particular in cancers having a high and/or dense stromal content. In cancers for which the quantitative content of fibroblasts can vary between patients, a convenient way to determine the amount of fibroblasts, that produce the components of desmoplastic stroma, is a determination of the stromal content. Cancers with a high stromal content, e.g. with a tumor-stroma ratio (TSR) of 40% or more, in particular of 50% or more, more in particular of 60% or more, even more in particular of 70% or more, are gastric cancer, colorectal cancer, cervical cancer, triple-negative breast cancer, head and neck cancer, non-small cell lung cancer, liver cancer, glioblastoma and esophageal cancer. In these cancers, a higher stromal content, i.e. a higher TSR, correlates with the amount of cancer-associated fibroblasts (CAFs) and a worse prognosis, and thus identify a suitable population for fibroblast-targeted therapies. As used herein, “tumors having a particular Tumor-Stroma Ratio (TSR)” refers to cancers having a high stromal content, in particular a cancer selected from the group consisting of gastric cancer, colorectal cancer, cervical cancer, triple-negative breast cancer, head and neck cancer, non-small cell lung cancer, liver cancer, glioblastoma and esophageal cancer.

The amount of stroma in a patient's tumor can be easily assessed by a person skilled in the art on haematoxylin-eosin (HE)-stained sections, and its assessment is clinically applied (Wu et al, 2016). The tumor-stroma ratio (TSR) is defined as the proportion of stroma relative to tumor cells and has been recognized as a prognostic factor for various solid tumors. For example, a TSR score of 20% corresponds to a section where 20% of the analyzed area consisted of stroma and 80% of the area consisted of tumor cells. The analysis of TSR thus can be easily replicated and routinely analyzed. The TSR was validated by international research groups and showed to be a robust parameter with good interobserver agreement. The UNITED study (Uniform Noting for International Application of the Tumor-stroma ratio as Easy Diagnostic tool) is being developed to bring the TSR to current guidelines for clinical implementation. In more detail, TSR is determined from invasive parts of tumor where the most stroma is present, as areas with a high amount of stroma are decisive for prognosis. As described in detail by Kemi et al. 2018, the part of tumor with most stroma is identified using a low magnification (total magnification 10-50×). A single vision field at a total magnification of 100× with tumor cells present at all four corners of the vision field is used for analysis. Excluded from the analysis are smooth muscle, mucin and necrosis. The remaining area consists of tumor cells and stroma and is used as the area under analysis. The area of stroma compared to the whole area under analysis is estimated and scored by 10% intervals using human assessment or computer-assisted analysis. As stated earlier, a score of 20% means that 20% of the area under analysis consists of stroma and 80% consists of tumor cells.

When using the TSR during diagnosis and therapy decision making, a 50% ratio is defined as the cut of point. Patients are divided into stroma-poor (proportion of stroma ≤50%) cases and stroma-rich (proportion of stroma >50%) cases. HE-stained TSR classes have been shown to distinguish stroma-rich, high-risk patients (TSR >50%) in gastric cancer, colorectal cancer, cervical cancer, triple-negative breast cancer, head and neck cancer, non-small cell lung cancer, liver cancer, glioblastoma and esophageal cancer. From the previous paragraphs, it follows that in these cancers, a TSR of >50% indicates patients with fibroblast-driven tumor progression, and thus a target population for treatment with the compounds of the present invention.

In addition to the above list of cancers, there are other cancer types in which (protumoral) CAFs are abundantly present regardless of the TSR status. These cancer types are pancreatic ductal adenocarcinoma (PDAC) and invasive lobular breast cancer (ILC). PDAC and ILC tumors show elevated levels of CAF markers such as platelet-derived growth factor receptor-beta, α-smooth muscle actin, fibroblast activation protein-α and fibroblast-specific protein 1/S100A4. Patients presenting with these cancers thus also form a target population for treatment with the compounds of the present invention.

An essential requirement for any CAF modulator to be of practical therapeutic use in human cancer patients is pharmacological selectivity for CAFs with respect to fibroblasts and other related cell types implicated in normal physiology. An example of such a related cell type are smooth muscle cells. Cancer-associated fibroblasts share a majority of druggable pathways and markers with normal fibroblasts and these related cell types. Normal fibroblasts fulfill an important role as structural components and in wound healing. They are critical in the immune response to a tissue injury: fibroblasts from different anatomical sites in the body express many genes that code for immune mediators and proteins. Smooth muscle cells contribute substantially to the regulation of blood pressure and blood flow to vascular beds, and to the phasic contraction of the digestive tract.

Accordingly, in a first aspect, the present invention provides a compound of Formula I or a salt, hydrate, or solvate thereof, for use in the treatment of invasive lobular breast carcinoma, tumors having a Tumor-Stroma Ratio (TSR) of >50%; and/or pancreatic ductal adenocarcinoma,

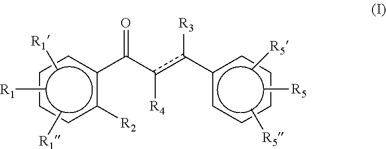

wherein
[symbol] represents a single or double bond;
each of said R₁, R₁′ and R₁″ is independently selected from the group consisting of —H, -halo, —CF₃, —OCF₃, and —OC₁₋₃alkyl;
R₂ is selected from the group consisting of —H, -halo, —CF₃, —OCF₃, and —OC₁₋₃alkyl;
R₃ is selected from the group consisting of —H, -halo, —C₁₋₃alkyl and —CF₃;
or R₂ and R₃ together with the C atoms to which they are attached form a 6-membered aromatic or non-aromatic heterocycle;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, —OC₁₋₃alkyl, —C₁₋₃alkyl-OH and —OC₁₋₃alkyl-OH;
wherein at least one of said R₁, R₁′ and R₁″ is —OC₁₋₃alkyl or —CF₃;
wherein when R₂ and R₃ do not form a heterocycle then [symbol] represents a double bond.

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 3 carbon atoms. Alkyl groups may be linear or branched and may be substituted as indicated herein. When a subscript is used herein following a carbon atom, the subscript refers to the number of carbon atoms that the named group may contain. C₁₋₃ alkyl includes all linear, branched, or cyclic alkyl groups with between 1 and 3 carbon atoms, and thus includes methyl, ethyl, n-propyl, c-propyl and i-propyl.

The term “optionally substituted alkyl” refers to an alkyl group optionally substituted with one or more substituents (for example 1 to 3 substituents, for example 1, 2, or 3 substituents or 1 to 2 substituents) at any available point of attachment. Non-limiting examples of such substituents includes oxo, amides, amines, aromatic and non-aromatic heterocycles and the like.

The term “halo” or “halogen” as a group or part of a group is generic for fluoro, chloro, bromo, or iodo. In a particular embodiment, halo is fluoro or chloro; preferably fluoro.

The terms “heterocyclyl”, “heterocyle” or “heterocyclo” as used herein by itself or as part of another group refer to aromatic or non-aromatic, cyclic groups (for example, 5 to 6 member monocyclic ring systems) which have from 1 to 3, such as 1, 2 or 3 heteroatoms in the carbon atom-containing ring. Said heteroatoms may be selected from nitrogen atoms, oxygen atoms and/or sulfur atoms, where the nitrogen and sulfur heteroatoms may optionally be oxidized and the nitrogen heteroatoms may optionally be quaternized. In a particular embodiment, said heteroatoms are selected from N and O atoms. The heterocyclic group may be attached at any heteroatom or carbon atom of the ring where valence allows. An optionally substituted heterocycle refers to a heterocycle having optionally one or more substituents (for example 1 to 3 substituents, or for example 1, 2, or 3), such as selected from —C₁₋₃alkyl, —OC₁₋₃alkyl, —C₁₋₃alkyl-OH and —OC₁₋₃alkyl-OH. Exemplary heterocyclic groups include piperazinyl and morpholinyl.

Where in the context of the present invention R₂ and R₃ together with the C atoms to which they are attached form a 6-membered aromatic or non-aromatic heterocycle, this results in the formation of a compound as defined by any one of formula (IIa) or (IIb):

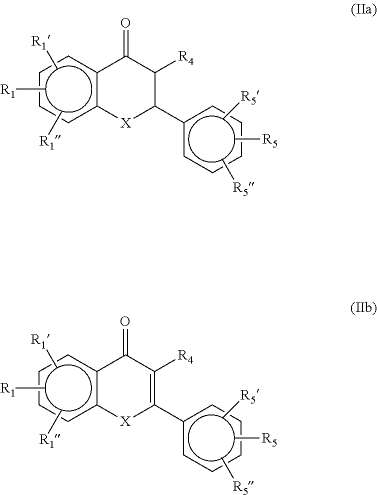

The term “oxo” as used herein refers to the group ═O.

The term “alkoxy” or “alkyloxy” as used herein refers to a radical having the Formula —OR^{b} wherein R^{b} is alkyl. Preferably, alkoxy is C₁₋₃ alkoxy, C₁₋₂ alkoxy, or methoxy. Non-limiting examples of suitable alkoxy include methoxy, ethoxy, propoxy, or isopropoxy.

The term “solvate” refers to an aggregate of a compound with one or more solvent molecules. As used herein, a “hydrate” is a compound formed by the hydration, i.e. addition of water or of the elements of water (i.e. H and OH) to a molecular entity. The compounds of this invention can further be provided in the form of pharmaceutically acceptable salts. As used herein, the term pharmaceutical acceptable salts or complexes refers to appropriate salts or complexes of the active compounds according to the present invention which retain the desired biological activity of the parent compound and exhibit limited toxicological effects to normal cells. Non limiting examples of such salts are acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others.

The compounds provided herein may be prepared using the reaction routes and synthesis schemes as described below, employing the techniques available in the art using starting materials that are readily available.

In a specific embodiment, the present invention provides a compound of Formula I or a salt, hydrate, or solvate thereof, for use in the treatment of invasive lobular breast carcinoma, tumors having a Tumor-Stroma Ratio (TSR) of >50%; and/or pancreatic ductal adenocarcinoma,

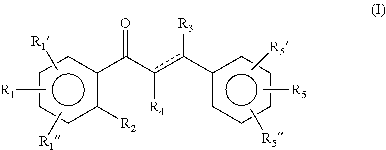

wherein one or more of the following applies:
[symbol] represents a single or double bond;
each of said R₁, R₁′ and R₁″ is independently selected from the group consisting of —H, -halo, —CF₃, —OCF₃, and —OC₁₋₃alkyl; in particular-H, —CF₃ and —OC₁₋₂alkyl; more in particular —H and —OCH₃;
R₂ is selected from the group consisting of —H, -halo, —CF₃, —OCF₃, and —OC₁₋₃alkyl; in particular —H;
R₃ is selected from the group consisting of —H, -halo, —C₁₋₃alkyl and —CF₃; in particular —H and -Me;
or R₂ and R₃ together with the C atoms to which they are attached form a 6-membered aromatic or non-aromatic heterocycle;
R₄ is selected from the group consisting of —H, -halo, —CF₃, —OCF₃, and —OC₁₋₃alkyl; in particular —H
each of said R₅, R₅′ and R₅″ is independently selected from the group consisting of —H, —OH —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁; in particular each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁; more in particular each of said R₅, R₅′ and R₅″ is independently selected from the group consisting of —H, —OH, and -halo, such as —Cl and —F;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr, in particular R₆ and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, and —OC₁₋₃alkyl, —C₁₋₃alkyl-OH and —OC₁₋₃alkyl-OH; in particular a 6-membered heterocycle having from 1 to 3 heteroatoms selected from O and N; wherein each of said Het₁ is optionally substituted with from 1 to 3 substituents selected from the group consisting of —C₁₋₂alyl, and —C₁₋₂alkyl-OH; more in particular morpholinyl and piperazinyl each optionally substituted with from 1 to 3 —C₁₋₂alkyl, and —C₁₋₂alkyl-OH,
wherein at least one of said R₁, R₁′ and R₁″ is —OC₁₋₃alkyl or —CF₃; in particular —OC₁₋₂alkyl; more in particular —OCH₃,
wherein when R₂ and R₃ do not form a heterocycle then [symbol] represents a double bond.

In a specific embodiment, the compounds for use as defined herein are those wherein:
when R₂ and R₃ form a 6-membered aromatic or non-aromatic heterocycle, then said compound is represented by formula (II)

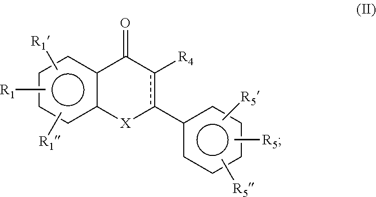

wherein X is selected from the group consisting of O, S, NH or N—C₁₋₃alkyl.

In another specific embodiment, the compounds, in particular of Formula I or II, for use as defined herein are those wherein:
[symbol] represents a single or double bond;
each of said R₁, R₁′ and R₁″ is independently selected from the group consisting of —H, —CF₃ and —OC₁₋₂alkyl;
R₂ is —H;
R₃ is selected from the group consisting of —H, and -Me;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆, and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is a 6-membered heterocycle having from 1 to 3 heteroatoms selected from O and N; wherein each of said Het₁ is optionally substituted with from 1 to 3 substituents selected from the group consisting of —C₁₋₂alkyl, and —C₁₋₂alkyl-OH;
wherein at least one of said R₁, R₁′ and R₁″ is —OC₁₋₂alkyl;
wherein when R₂ and R₃ do not form a heterocycle then [symbol] represents a double bond.

In yet a further specific embodiment, the compounds, in particular of Formula I or II, for use as defined herein are those wherein:
[symbol] represents a single or double bond;
each of said R₁, R₁′ and R₁″ is independently selected from the group consisting of —H, —CF₃ and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆ and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is selected from morpholinyl and piperazinyl; wherein each of said Het₁ is optionally substituted with from 1 to 3 substituents selected from the group consisting of —C₁₋₂alkyl, and —C₁₋₂alkyl-OH;
wherein at least one of said R₁, R₁′ and R₁″ is —OCH₃;
wherein when R₂ and R₃ do not form a heterocycle then [symbol] represents a double bond.

In a further embodiment, the present invention provides a compound, in particular of Formula I or II, for use as defined herein wherein:
[symbol] represents a single or double bond;
each of said R₁, R₁′ and R₁″ is independently selected from the group consisting of —H, - and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, and -halo;
wherein at least one of said R₁, R₁′ and R₁″ is —OCH₃;
wherein when R₂ and R₃ do not form a heterocycle then [symbol] represents a double bond.

In yet a further embodiment, the present invention provides a compound, in particular of Formula I or II, for use as defined herein wherein:
each of said R₁, R₁′ and R₁″ is independently selected from the group consisting of —H—and —OCH₃;
R₂ is —H;
R₃ is —H;
or R₂ and R₃ together with the C atoms to which they are attached form a 6-membered aromatic or non-aromatic heterocycle, R₄ is —H;
each of said R₅, R₅′ and R₅″ is independently selected from the group consisting of —H, —OH, —F and —Cl;
wherein at least one of said R₁, R₁′ and R₁″ is —OCH₃;
wherein when R₂ and R₃ do not form a heterocycle then [symbol] represents a double bond.

It was moreover found that combinations of one or more methoxy groups on one aromatic ring of the molecule and one or more halogens, with or without a hydroxyl group, on the other aromatic ring of the molecule, are advantageous for the desired activity.

Accordingly, the present invention provides a compound of Formula I or II for use as defined herein wherein:
each of said R₁, R₁′ and R₁″ is independently selected from the group consisting of —H—and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —F and —Cl;
wherein at least one of said R₁, R₁′ and R₁″ is —OCH₃; and wherein R₅ is —OH.
wherein when R₂ and R₃ do not form a heterocycle then [symbol] represents a double bond.

The compounds for use as defined herein may in a specific embodiment be represented by any one of formula (Ia), (Ila) or (IIb)), including the R-groups and substituents as defined herein for Formulas I and II resp.

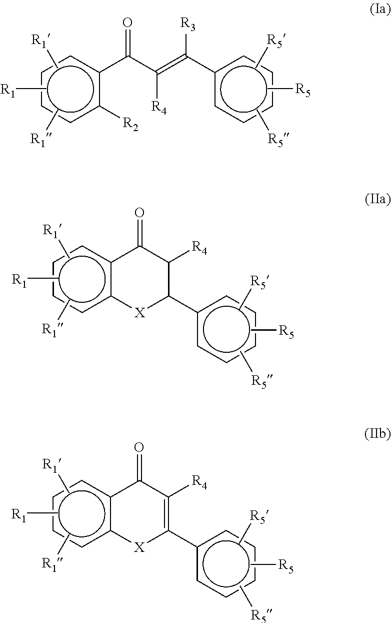

The present invention also provides a compound or a salt, hydrate, or solvate thereof, for use as defined herein, wherein said compound is selected from the list comprising:

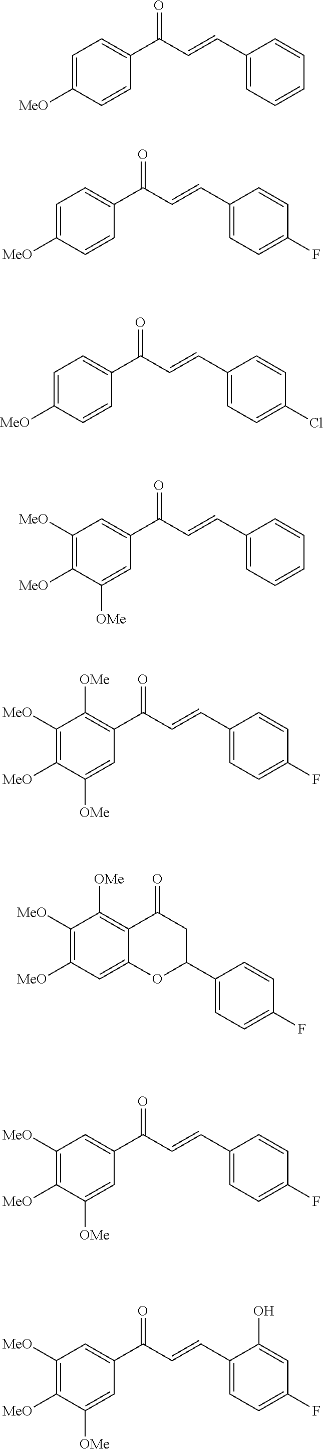

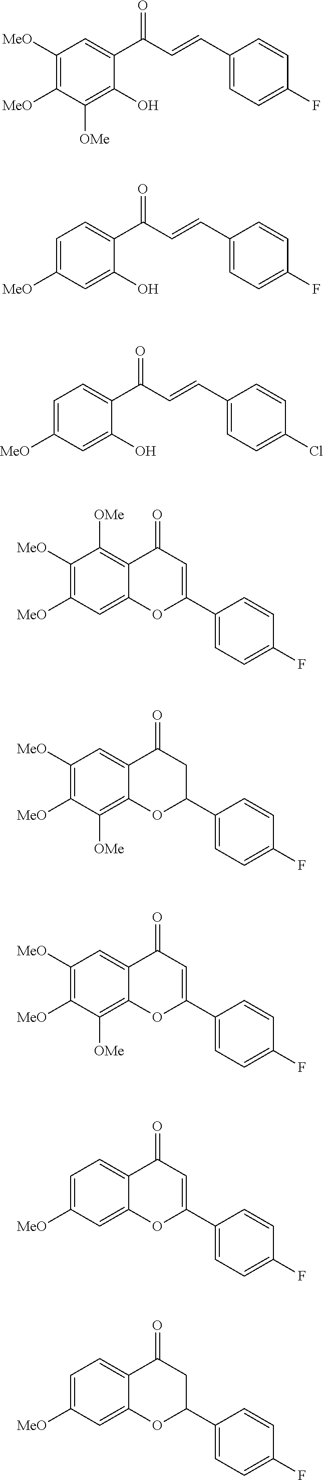

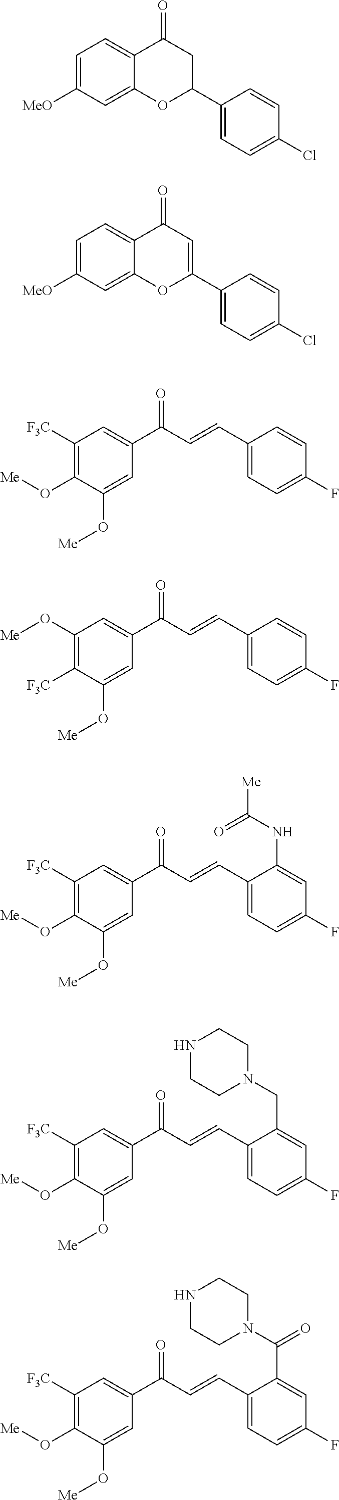

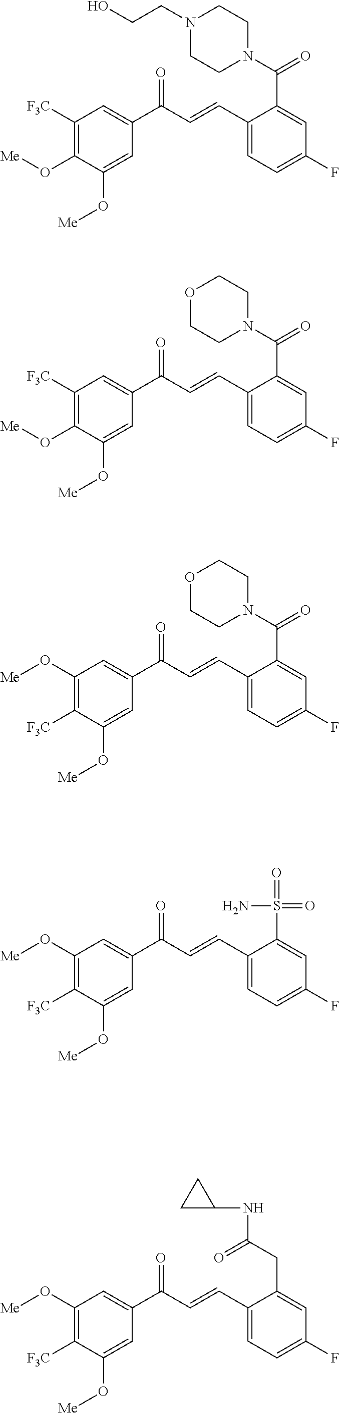 [SMILES: COc1cc(C(=O)/C=C/c2ccc(F)cc2C(=O)N2CCN(CCO)CC2)cc(C(F)(F)F)c1OC ; COc1cc(C(=O)/C=C/c2ccc(F)cc2C(=O)N2CCOCC2)cc(C(F)(F)F)c1OC ; COc1cc(C(=O)/C=C/c2ccc(F)cc2C(=O)N2CCOCC2)cc(OC)c1C(F)(F)F ; COc1cc(C(=O)/C=C/c2ccc(F)cc2S(N)(=O)=O)cc(OC)c1C(F)(F)F ; COc1cc(C(=O)/C=C/c2ccc(F)cc2CC(=O)NC2CC2)cc(OC)c1C(F)(F)F]

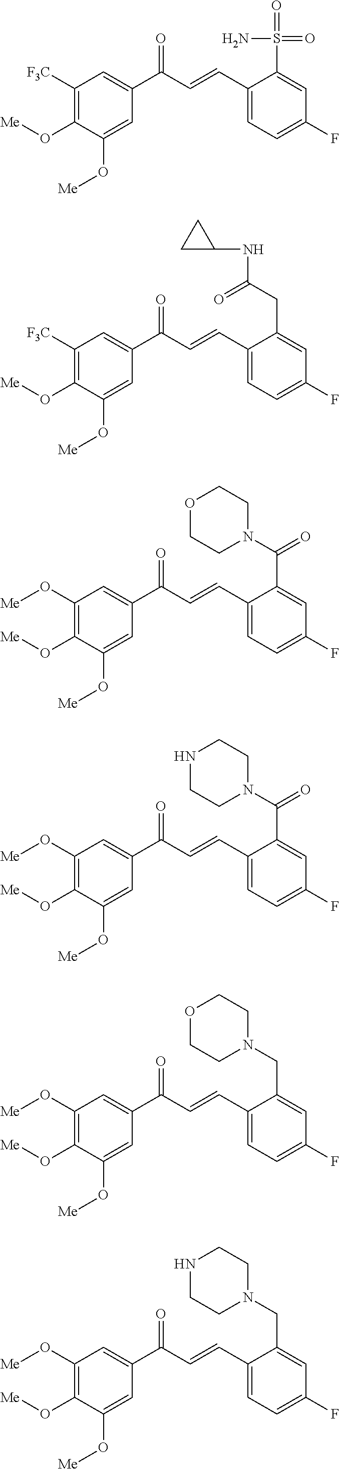 [SMILES: COc1cc(C(=O)/C=C/c2ccc(F)cc2S(N)(=O)=O)cc(C(F)(F)F)c1OC ; COc1cc(C(=O)/C=C/c2ccc(F)cc2CC(=O)NC2CC2)cc(C(F)(F)F)c1OC ; COc1cc(C(=O)/C=C/c2ccc(F)cc2C(=O)N2CCOCC2)cc(OC)c1OC ; COc1cc(C(=O)/C=C/c2ccc(F)cc2C(=O)N2CCNCC2)cc(OC)c1OC ; COc1cc(C(=O)/C=C/c2ccc(F)cc2CN2CCOCC2)cc(OC)c1OC ; COc1cc(C(=O)/C=C/c2ccc(F)cc2CN2CCNCC2)cc(OC)c1OC]

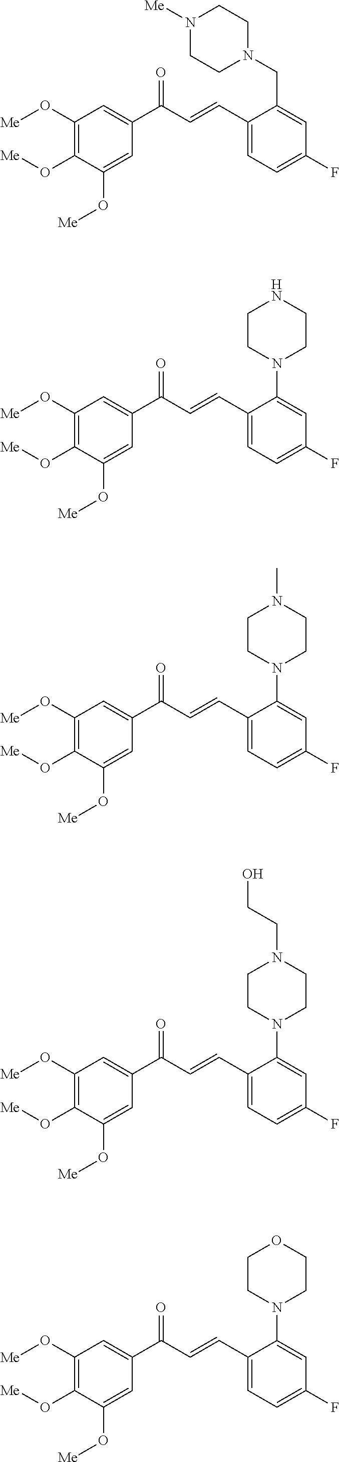 [SMILES: COc1cc(C(=O)/C=C/c2ccc(F)cc2CN2CCN(C)CC2)cc(OC)c1OC ; COc1cc(C(=O)/C=C/c2ccc(F)cc2N2CCNCC2)cc(OC)c1OC ; COc1cc(C(=O)/C=C/c2ccc(F)cc2N2CCN(C)CC2)cc(OC)c1OC ; COc1cc(C(=O)/C=C/c2ccc(F)cc2N2CCN(CCO)CC2)cc(OC)c1OC ; COc1cc(C(=O)/C=C/c2ccc(F)cc2N2CCOCC2)cc(OC)c1OC]

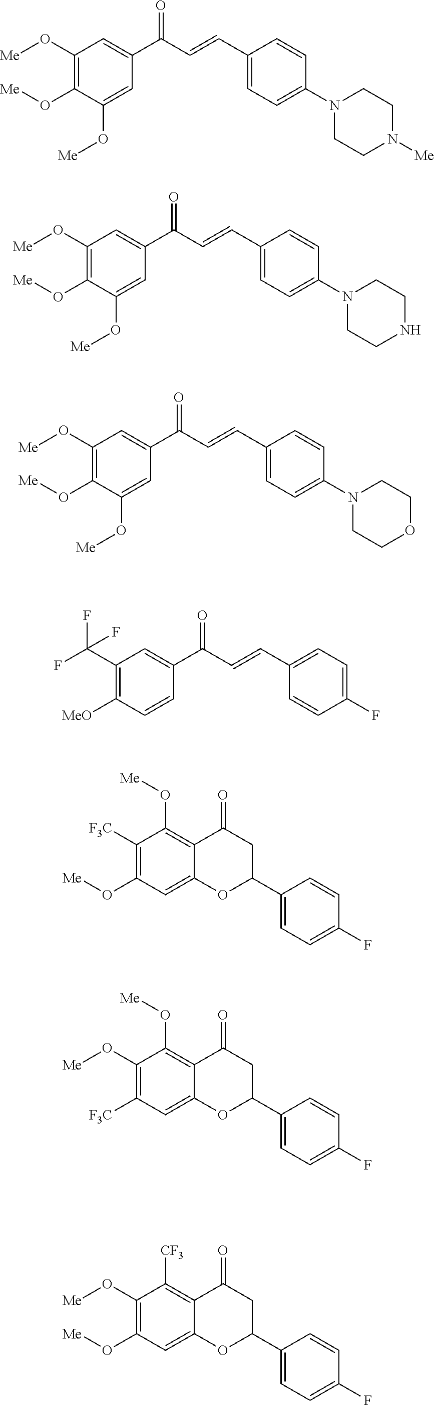

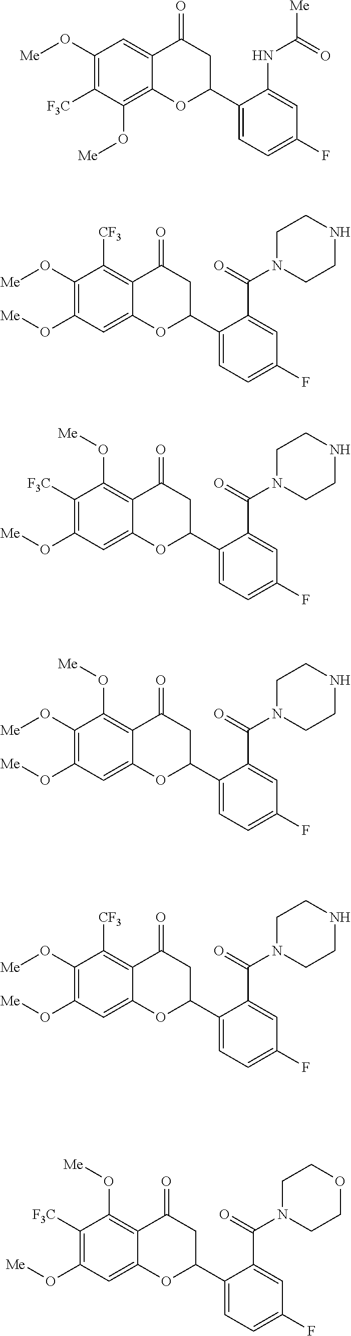 [SMILES: COc1cc2c(c(OC)c1C(F)(F)F)OC(c1ccc(F)cc1NC(C)=O)CC2=O ; COc1cc2c(c(C(F)(F)F)c1OC)C(=O)CC(c1ccc(F)cc1C(=O)N1CCNCC1)O2 ; COc1cc2c(c(OC)c1C(F)(F)F)C(=O)CC(c1ccc(F)cc1C(=O)N1CCNCC1)O2 ; COc1cc2c(c(OC)c1OC)C(=O)CC(c1ccc(F)cc1C(=O)N1CCNCC1)O2 ; COc1cc2c(c(C(F)(F)F)c1OC)C(=O)CC(c1ccc(F)cc1C(=O)N1CCNCC1)O2 ; COc1cc2c(c(OC)c1C(F)(F)F)C(=O)CC(c1ccc(F)cc1C(=O)N1CCOCC1)O2]

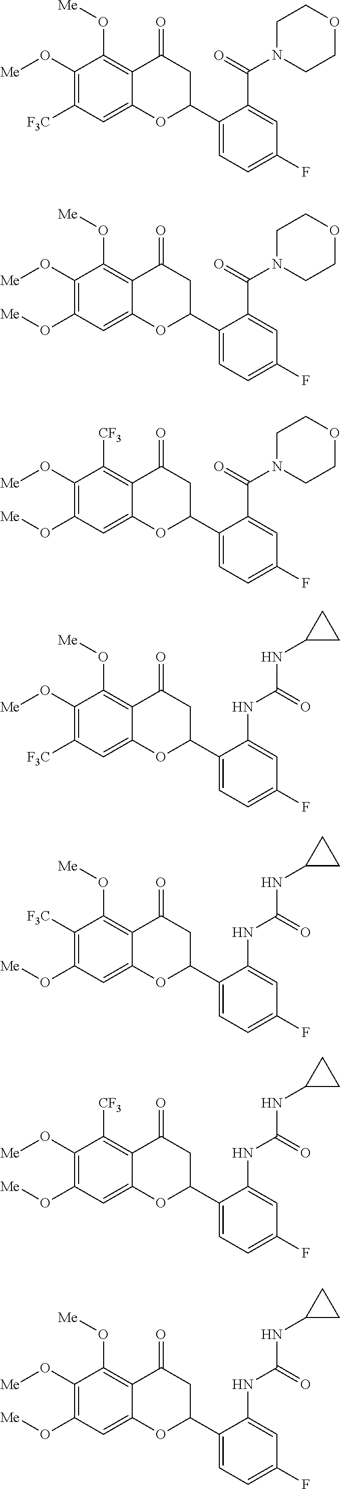

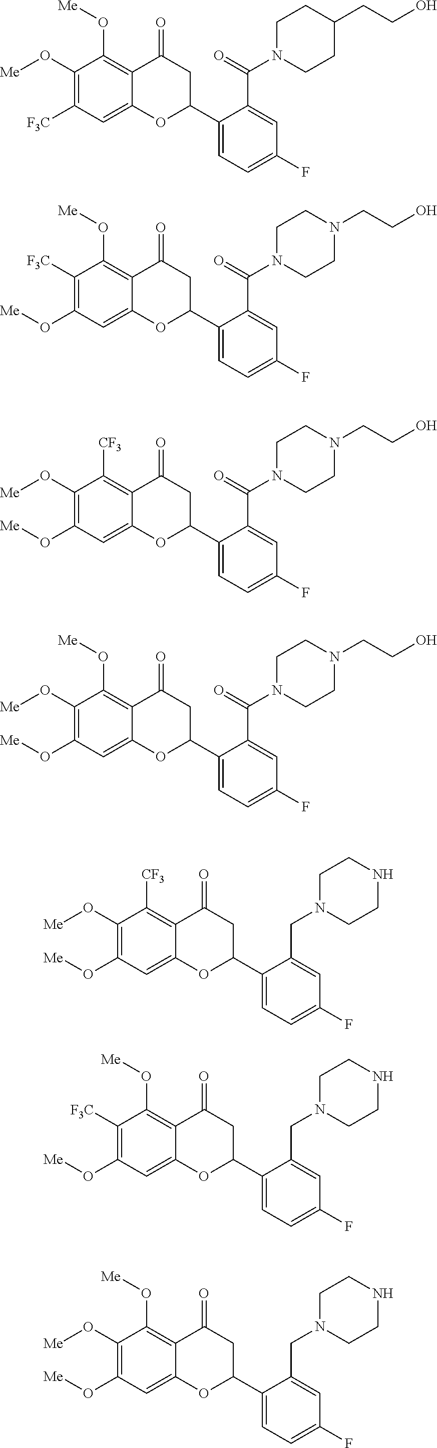

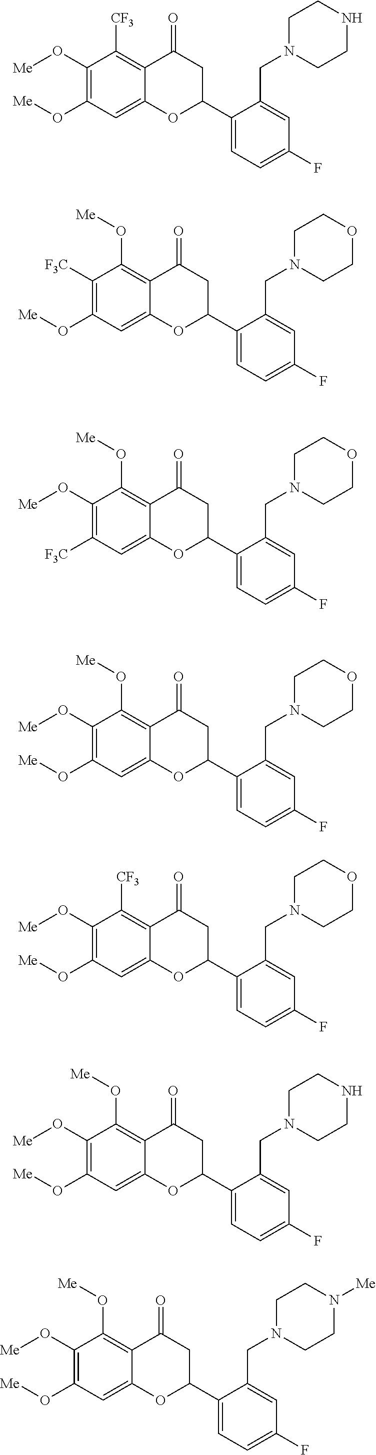

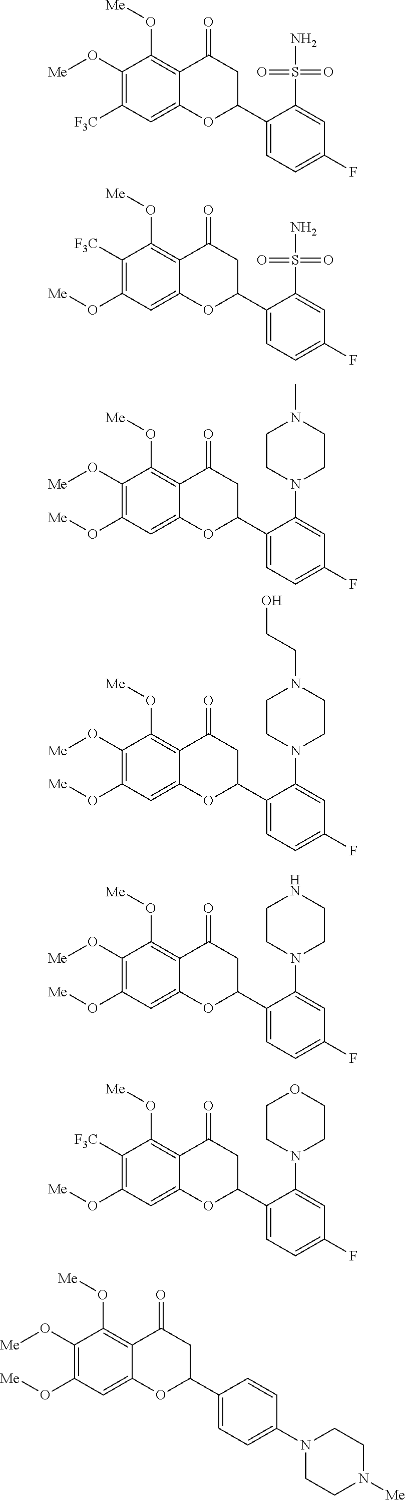

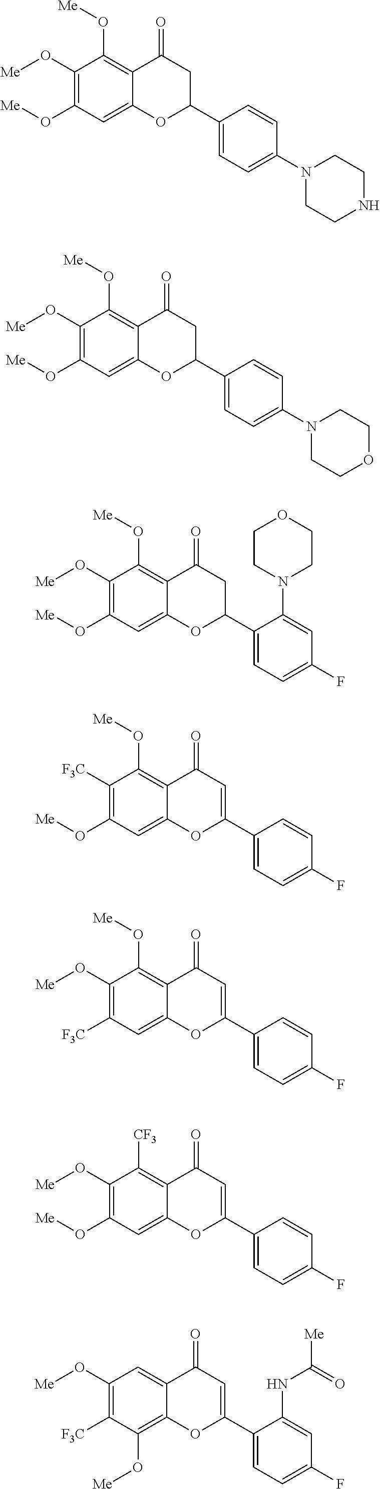

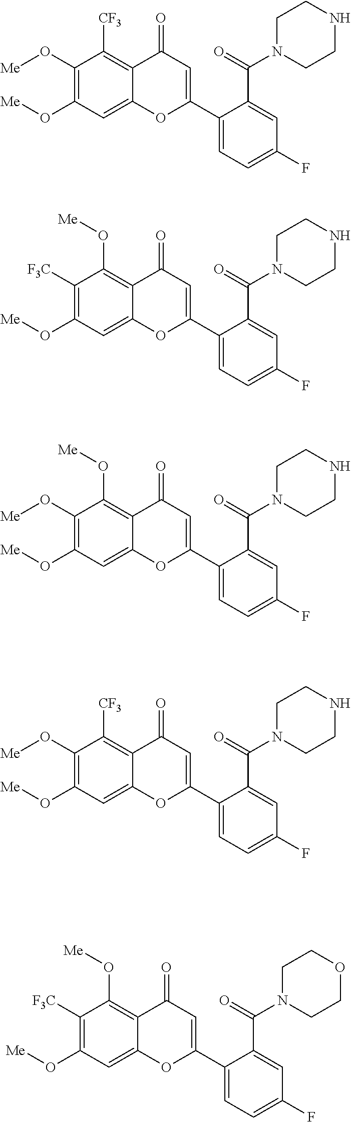 [SMILES: COc1cc2oc(-c3ccc(F)cc3C(=O)N3CCNCC3)cc(=O)c2c(C(F)(F)F)c1OC ; COc1cc2oc(-c3ccc(F)cc3C(=O)N3CCNCC3)cc(=O)c2c(OC)c1C(F)(F)F ; COc1cc2oc(-c3ccc(F)cc3C(=O)N3CCNCC3)cc(=O)c2c(OC)c1OC ; COc1cc2oc(-c3ccc(F)cc3C(=O)N3CCNCC3)cc(=O)c2c(C(F)(F)F)c1OC ; COc1cc2oc(-c3ccc(F)cc3C(=O)N3CCOCC3)cc(=O)c2c(OC)c1C(F)(F)F]

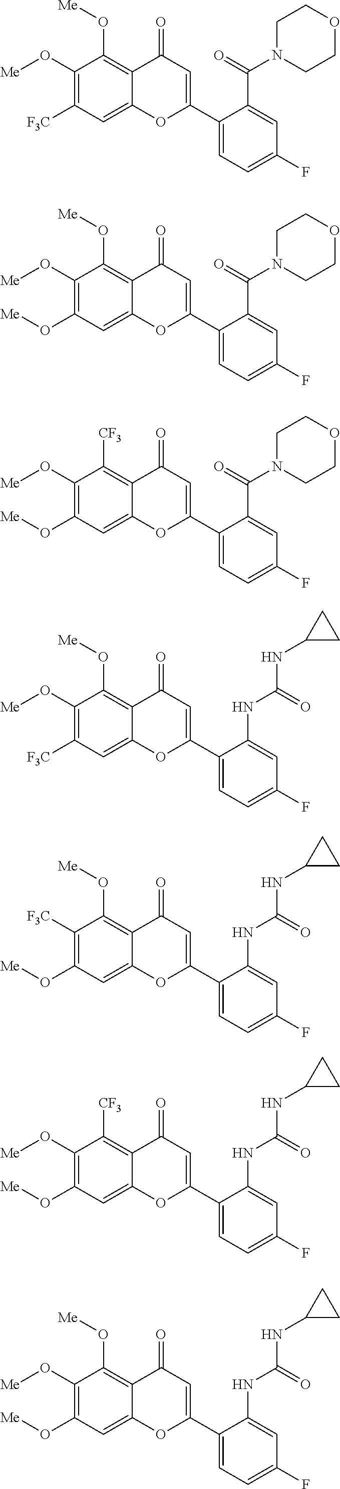

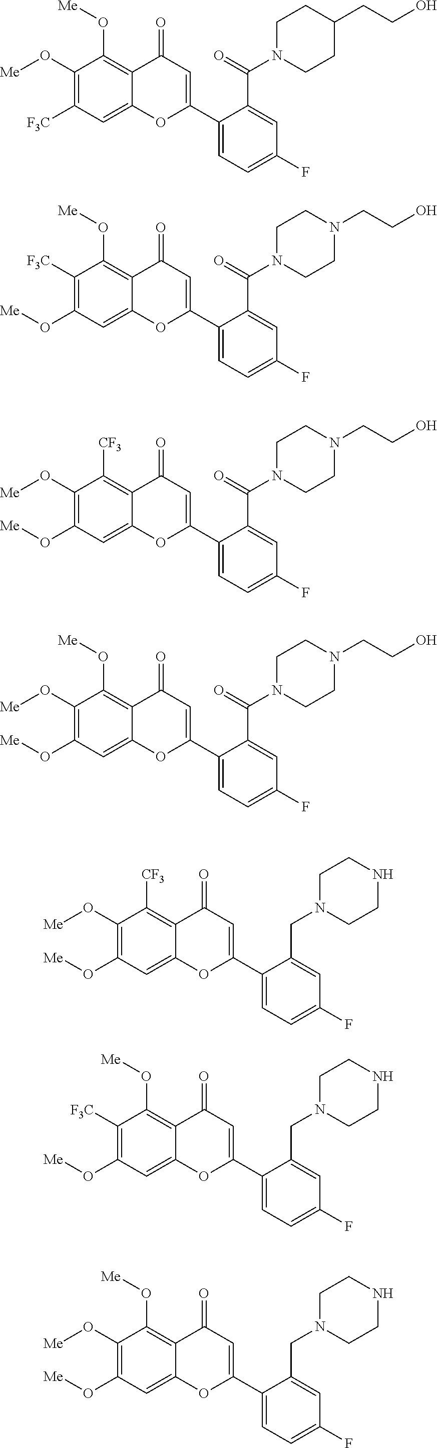

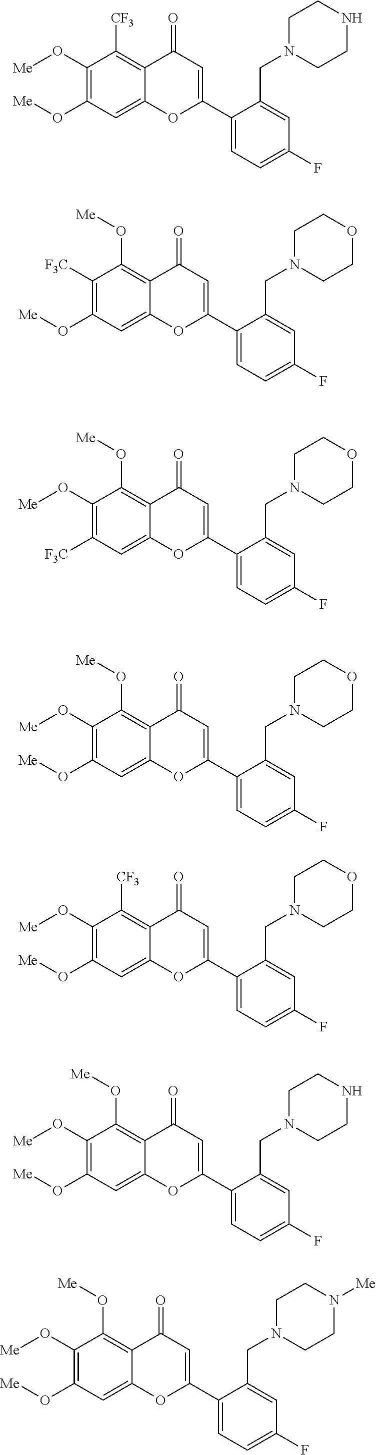

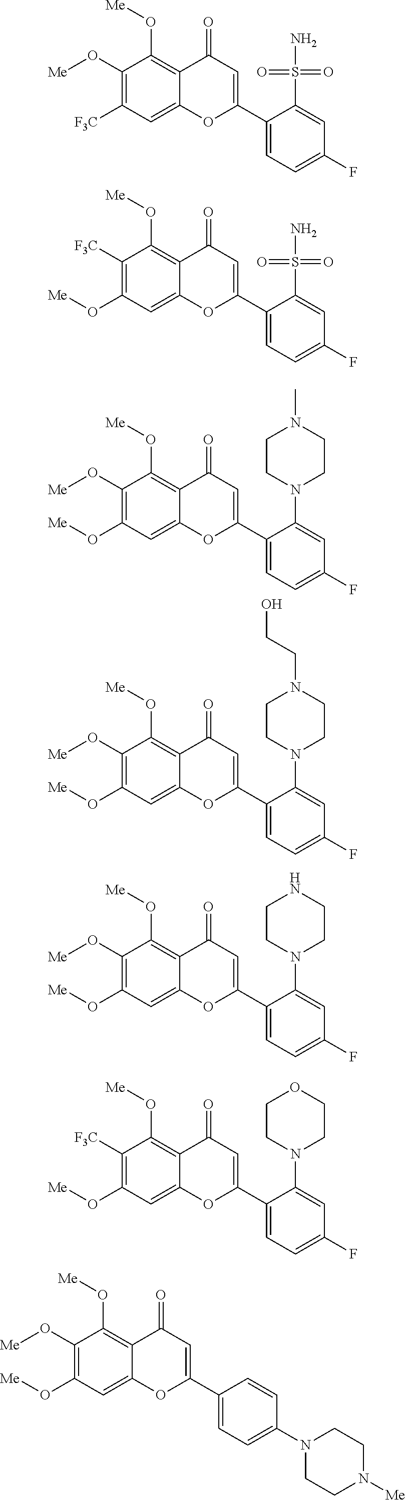

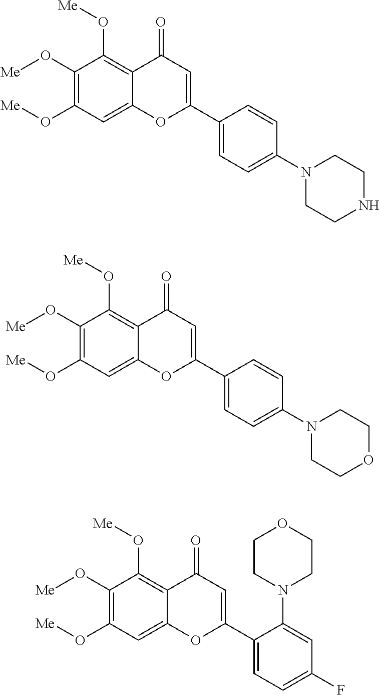 [SMILES: COc1cc2oc(-c3ccc(N4CCNCC4)cc3)cc(=O)c2c(OC)c1OC ; COc1cc2oc(-c3ccc(N4CCOCC4)cc3)cc(=O)c2c(OC)c1OC ; COc1cc2oc(-c3ccc(F)cc3N3CCOCC3)cc(=O)c2c(OC)c1OC]

In another aspect, the present invention provides a compound or a salt, hydrate, or solvate thereof, wherein the compound is selected from the list comprising:

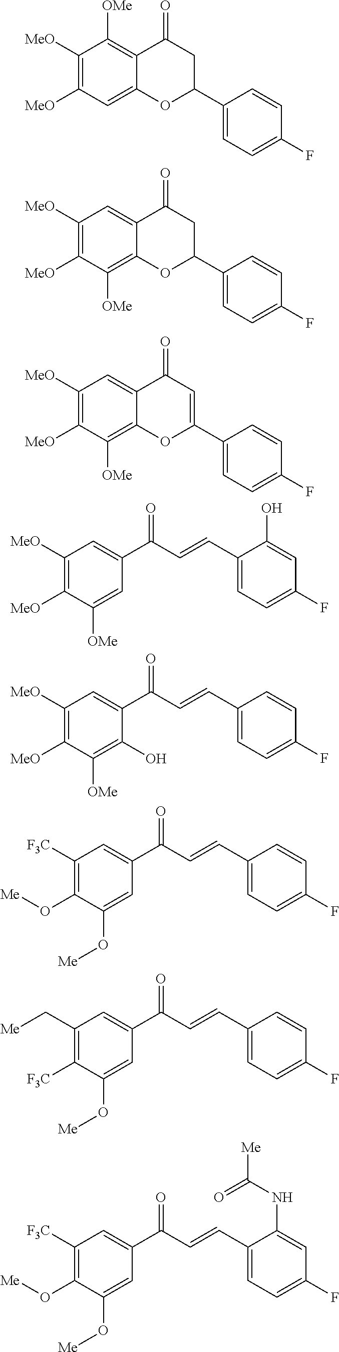

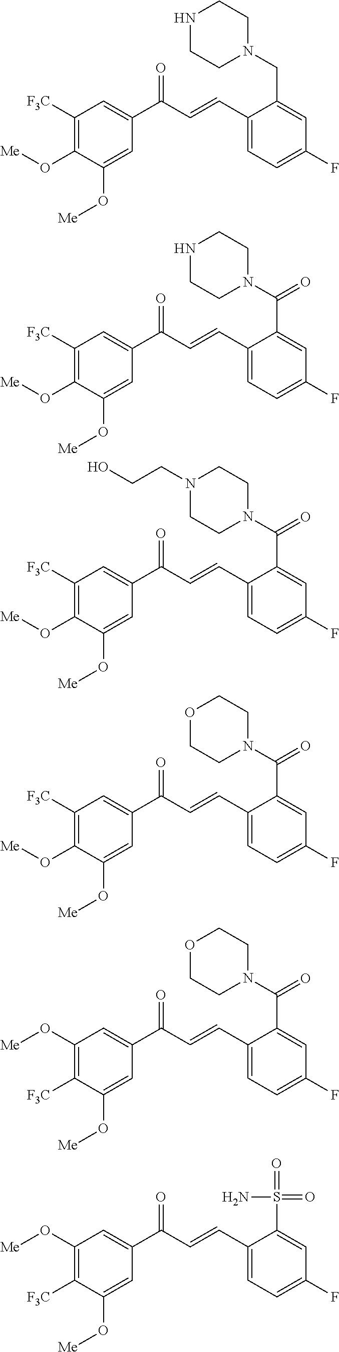

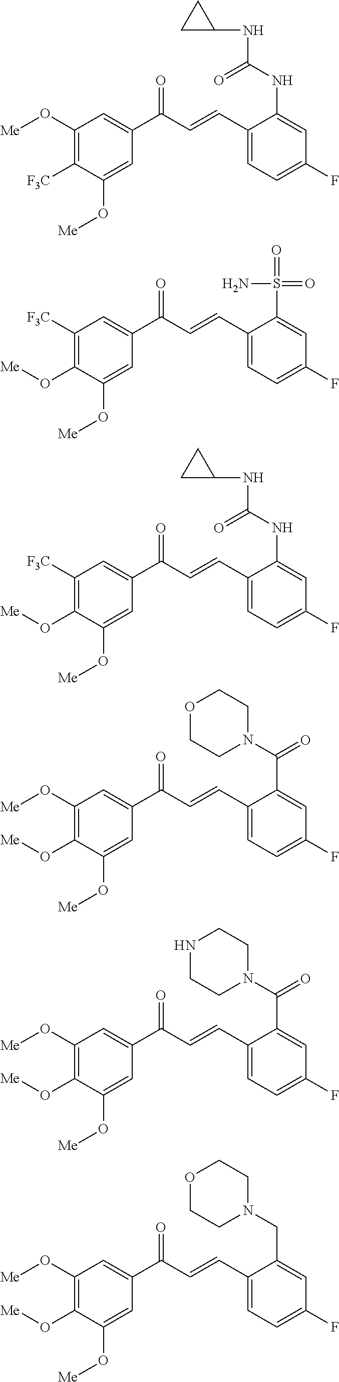 [SMILES: COc1cc(C(=O)/C=C/c2ccc(F)cc2NC(=O)NC2CC2)cc(OC)c1C(F)(F)F ; COc1cc(C(=O)/C=C/c2ccc(F)cc2S(N)(=O)=O)cc(C(F)(F)F)c1OC ; COc1cc(C(=O)/C=C/c2ccc(F)cc2NC(=O)NC2CC2)cc(C(F)(F)F)c1OC ; COc1cc(C(=O)/C=C/c2ccc(F)cc2C(=O)N2CCOCC2)cc(OC)c1OC ; COc1cc(C(=O)/C=C/c2ccc(F)cc2C(=O)N2CCNCC2)cc(OC)c1OC ; COc1cc(C(=O)/C=C/c2ccc(F)cc2CN2CCOCC2)cc(OC)c1OC]

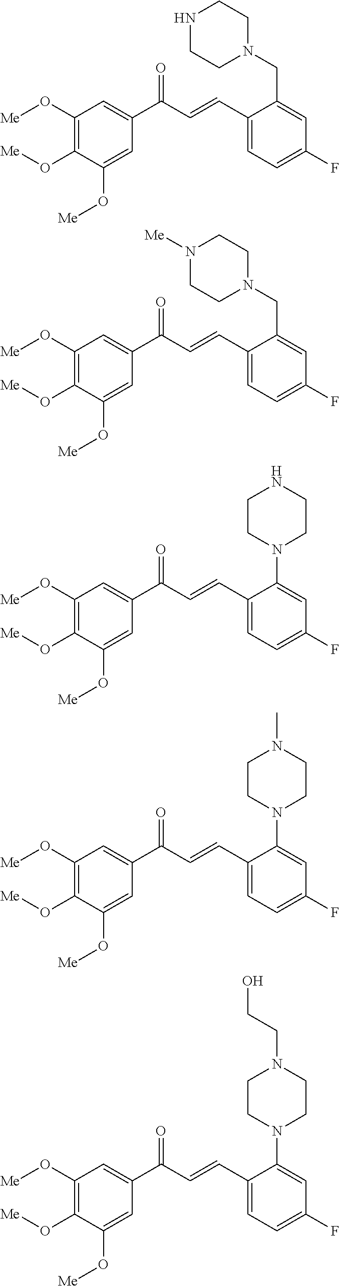 [SMILES: COc1cc(C(=O)/C=C/c2ccc(F)cc2CN2CCNCC2)cc(OC)c1OC ; COc1cc(C(=O)/C=C/c2ccc(F)cc2CN2CCN(C)CC2)cc(OC)c1OC ; COc1cc(C(=O)/C=C/c2ccc(F)cc2N2CCNCC2)cc(OC)c1OC ; COc1cc(C(=O)/C=C/c2ccc(F)cc2N2CCN(C)CC2)cc(OC)c1OC ; COc1cc(C(=O)/C=C/c2ccc(F)cc2N2CCN(CCO)CC2)cc(OC)c1OC]

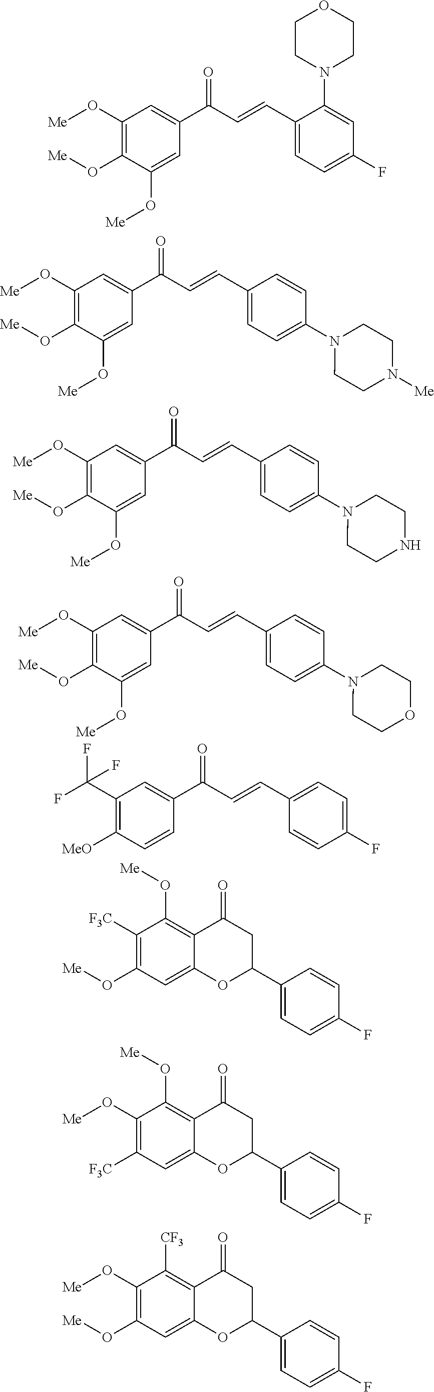

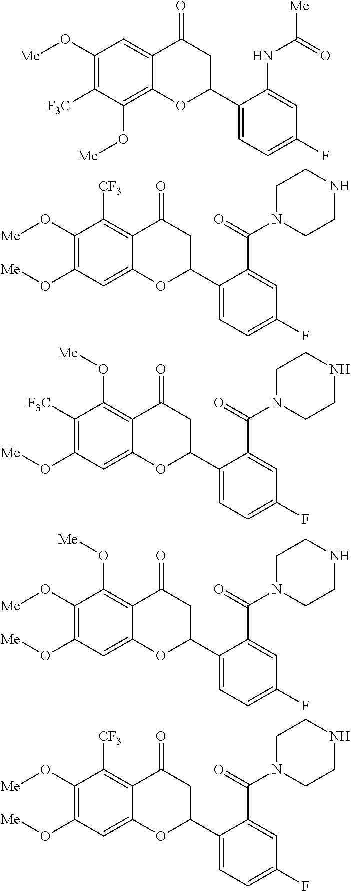

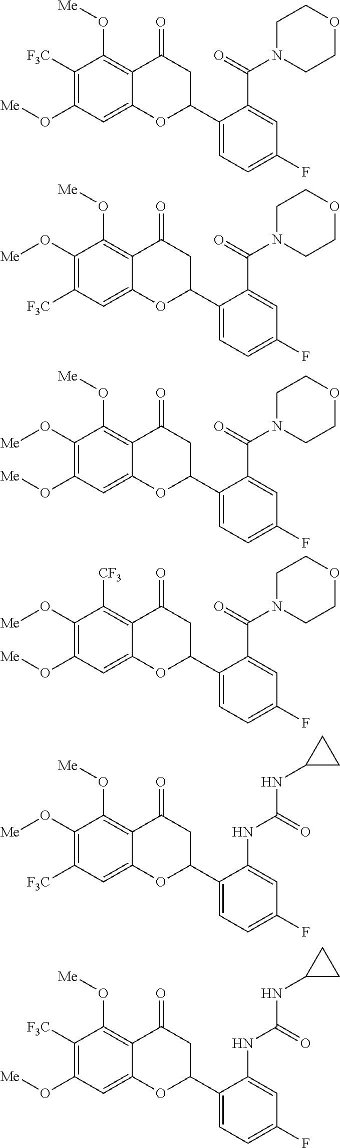

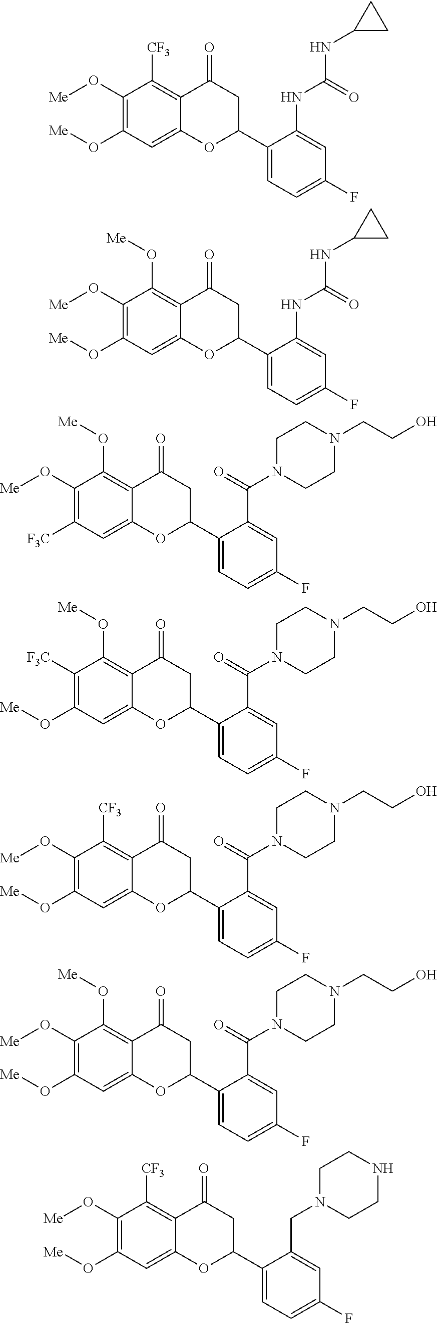

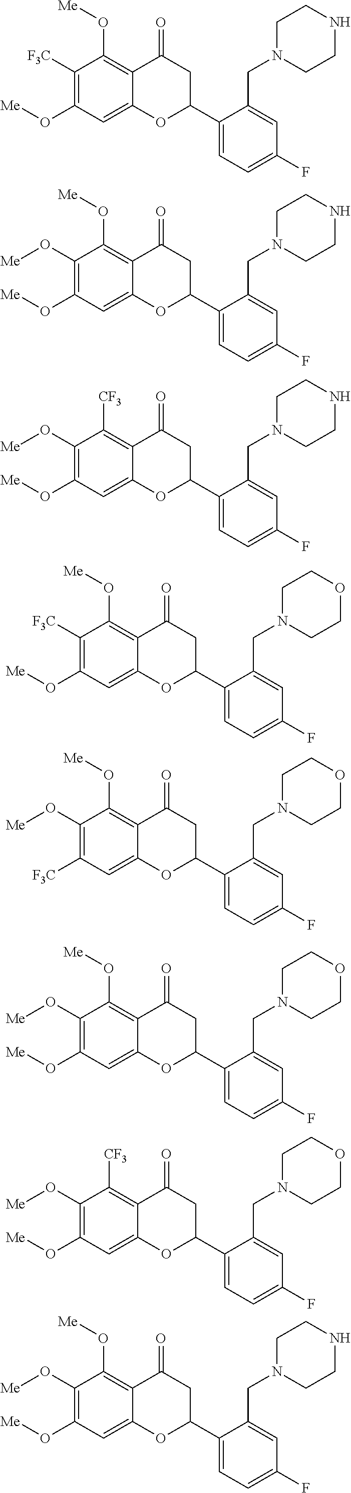

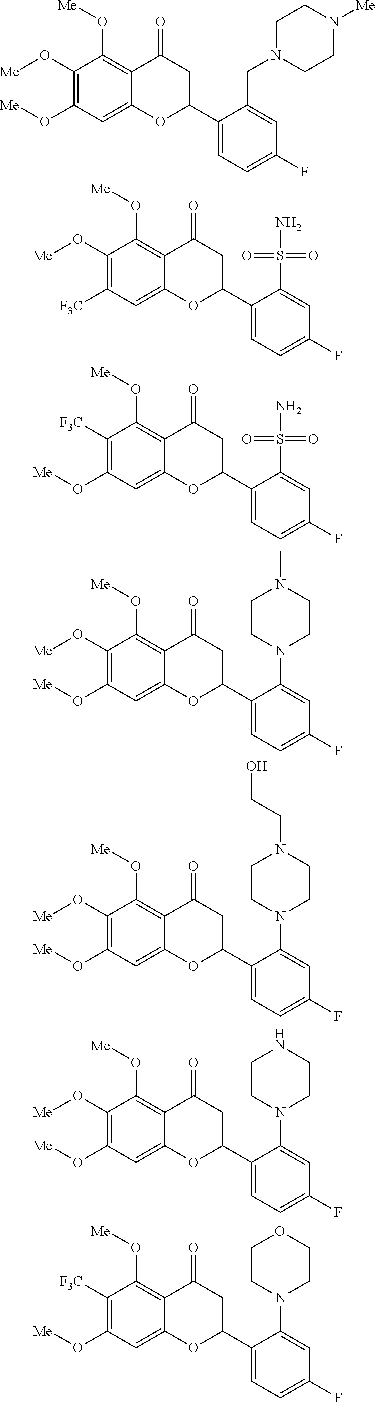

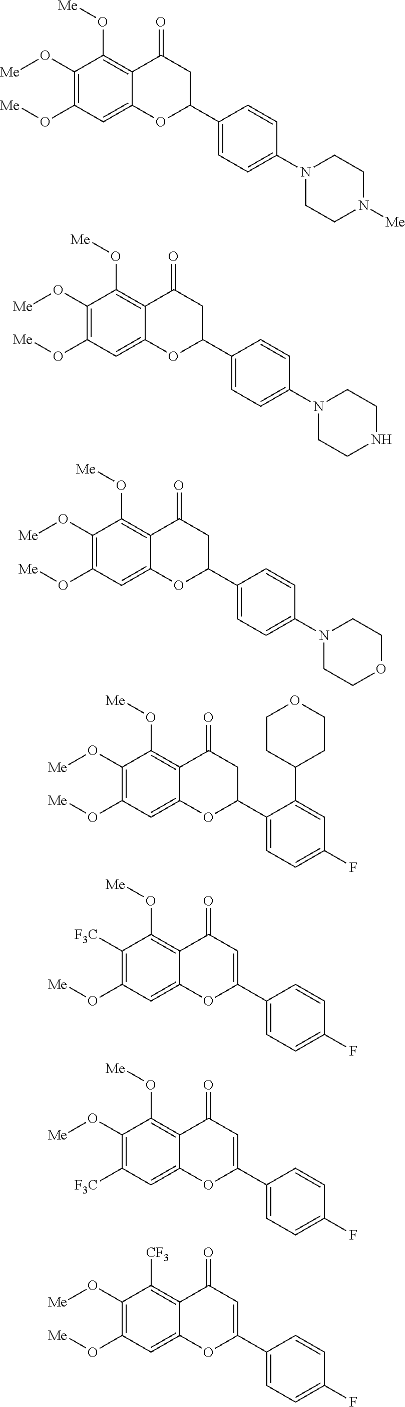

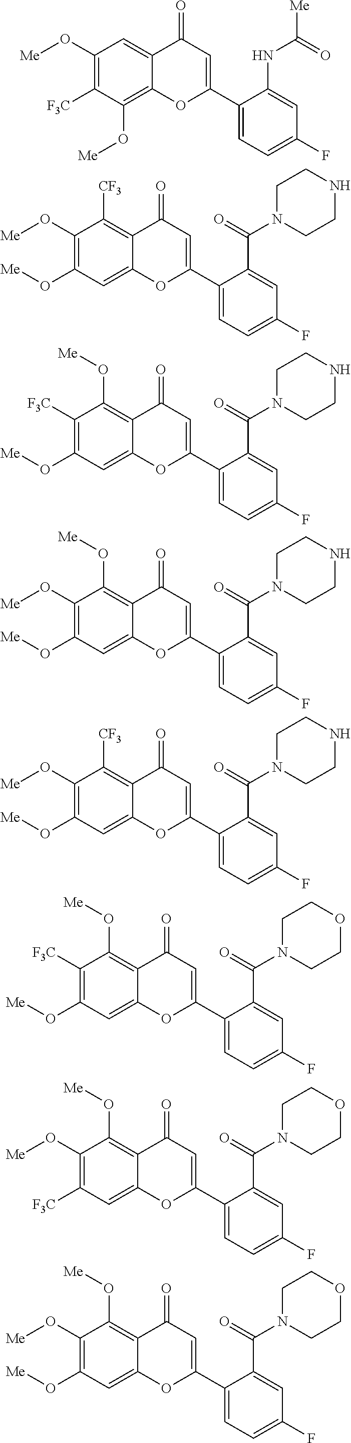

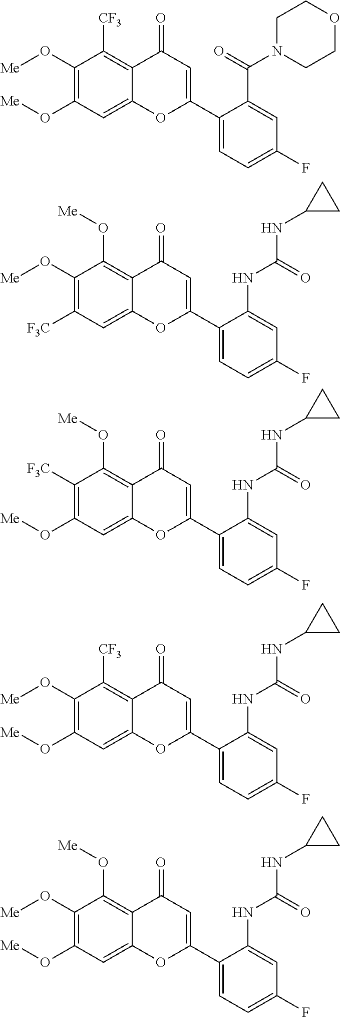

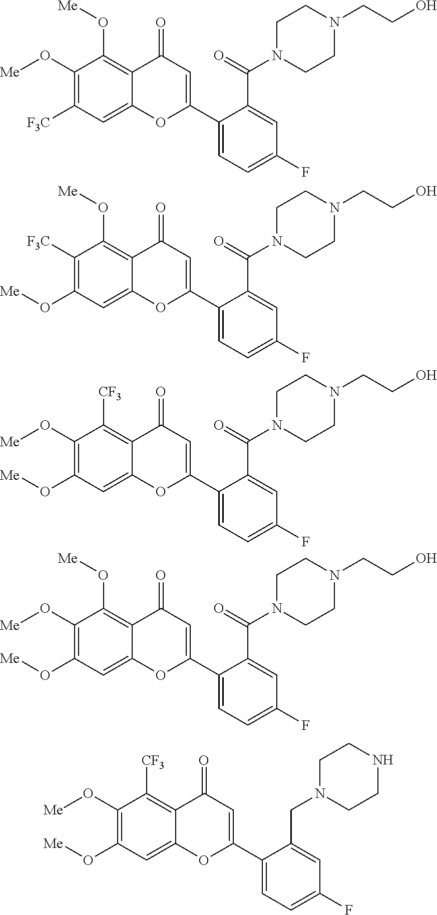

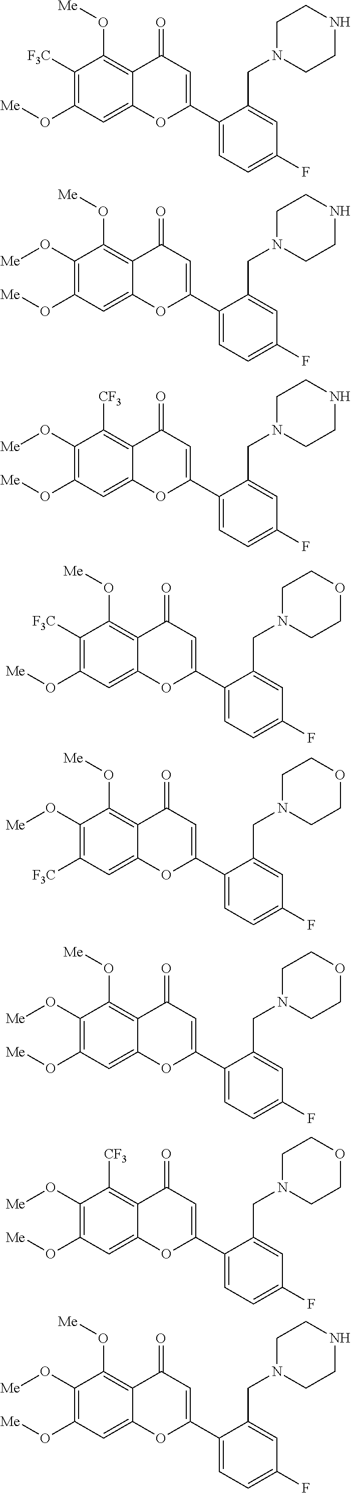

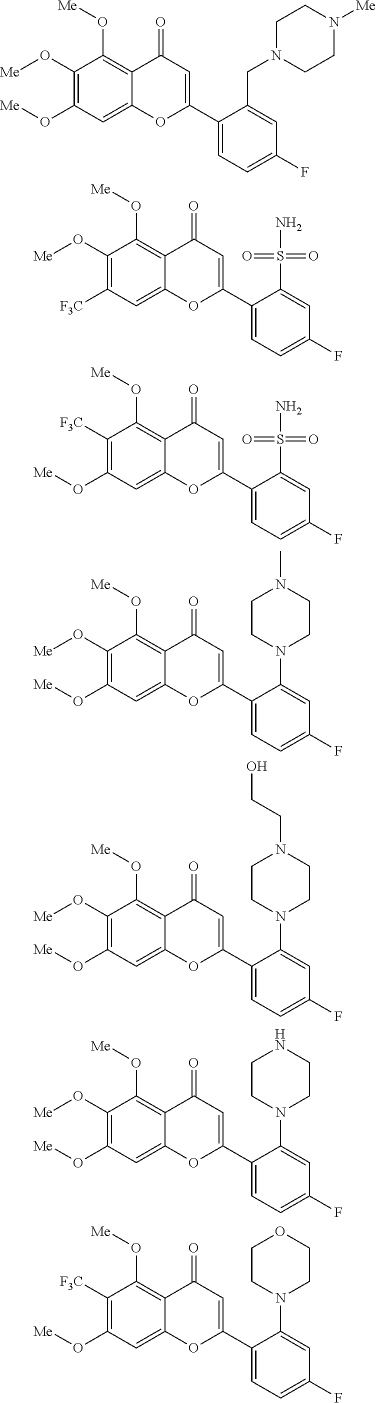

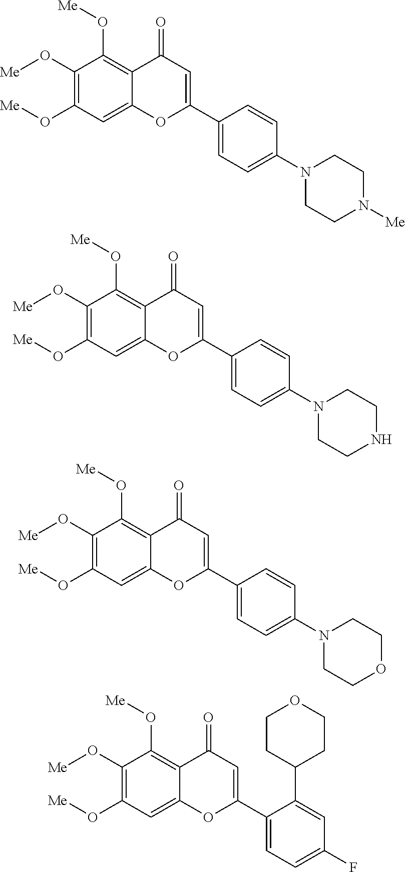

In a very specific embodiment, the present invention provides a compound or a salt, hydrate, or solvate thereof, wherein the compound is selected from the list comprising:

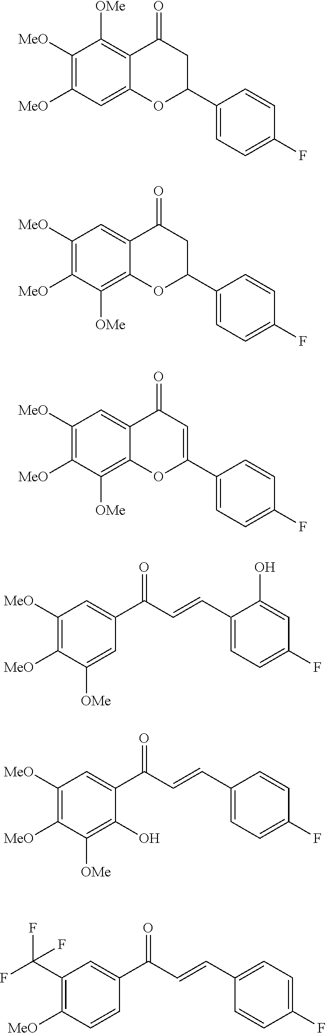

In order to use a compound of the formulas as provided herein for the therapeutic treatment (including prophylactic treatment) of a subject, such as an animal or a human, it is normally formulated in accordance with standard pharmaceutical practice as a pharmaceutical composition. The present invention hence also provides a pharmaceutical composition comprising a compound as defined herein and a pharmaceutically acceptable excipient.

The compounds of this invention may be incorporated into formulations for all routes of administration including for example, oral, topical and parenteral including intravenous, intramuscular, eye or ocular, intraperitoneal, intrabuccal, transdermal and in suppository form. In general, it is preferable to administer the pharmaceutical composition in orally administrable form, but for treatment of a number of conditions, a number of other formulations may be administered via a topical, parenteral, intravenous, intramuscular, transdermal, buccal, subcutaneous, suppository or other route, including an eye or ocular route. Intravenous and intramuscular formulations are preferably administered in sterile saline. Small modifications of the present compounds to render them more soluble in water or other vehicle, for example, may be easily accomplished by minor modifications (salt formulation, esterification, etc.) which are well known to skilled person in the art. It is also well within the skill of those skilled in the art to modify the route of administration and dosage regimen of a particular compound in order to manage the pharmacokinetics of the present compounds for maximum beneficial effect to the patient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Aqueous suspensions generally contain the compound in finely powdered form together with one or more suspending agents, such as sodium carboxymethylcellulose. methylcellulose, hydroxypropylmethylcellulose, sodium alginate, polyvinyl-pyrrolidone, gum tragacanth and gum acacia; dispersing or wetting agents such as lecithin or condensation products of an alkylen oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Additional excipients such as sweetening, flavoring and coloring agents, may also be present in the pharmaceutical composition.

The pharmaceutical composition of the invention may also be in the form of an oil-in-water emulsion.

The pharmaceutical composition may also be in the form of a sterile injectable aqueous or oily suspension, which may be formulated according to known procedures using one or more of the appropriate dispersing or wetting agents and suspending agents. A sterile injectable preparation may also be a sterile injectable solution or suspension in a non-toxic parenterally-acceptable diluent or solvent.

For example, a formulation intended for oral administration to humans may contain, for example, from 0.5 mg to 2 g of the compound of the invention with an appropriate and convenient amount of excipients which may vary from about 5 to about 98 percent by weight of the total composition. Dosage unit forms will generally contain about 1 mg to about 500 mg of the active compound. Suitable information on routes of administration and dosage regimes are provided e.g. in Chapter 25.3 in Volume 5 of Comprehensive Medicinal Chemistry (Corwin Hansch; Chairman of Editorial Board), Pergamon Press 1990, which is incorporated herein by reference.

The size of the dose for therapeutic or prophylactic purposes of a compound of the formulas presented herein will naturally vary according to the nature and severity of the conditions, the age and sex of the animal or patient and the route of administration, according to well known principles of medicine.

In a further embodiment, the present invention provides a compound or a composition as defined herein; for use in human or veterinary medicine; in particular for use in the treatment of cancer; more particular for use in preventing, treating or reducing tumor associated desmoplasia, even more in particular for use in the treatment of invasive lobular breast carcinoma, tumors having a Tumor-Stroma Ratio (TSR) of >50%; and/or pancreatic ductal adenocarcinoma.

The present invention also provides a method for the prevention and/or treatment of cancer; more particular for use in preventing, treating or reducing tumor associated desmoplasia, even more in particular for the treatment of invasive lobular breast carcinoma, tumors having a Tumor-Stroma Ratio (TSR) of >50%; and/or pancreatic ductal adenocarcinoma; said method comprising administering to a subject in need thereof a therapeutically effective amount of a compound or a pharmaceutical composition as defined herein.

In a further embodiment, the compounds of the invention are of particular interest to use in combination therapies for cancer, in particular in combination with cytotoxic agents or immune modulating agents, in particular with immune cell activating agents, such as immune checkpoint inhibitors (for example PD1 or PDL1 pathway inhibitors; in particular anti-PD1 or anti-PDL1 antibodies). Said combination therapy may be administered to the patient separately from the compounds of the invention, or simultaneously, whichever is most appropriate.

In another embodiment, compounds of the formula IIa and IIb have been demonstrated herein to exert anti-invasive activity, more specific these compounds inhibit invasion of tumor cells into the surrounding tissue thereby reducing (the risk of) metastasis.

Accordingly in yet a further aspect, the present invention provides a compound for use as an anti-invasive compound of tumor cells; said compound being represented by any one of formula (IIa) or (IIb)

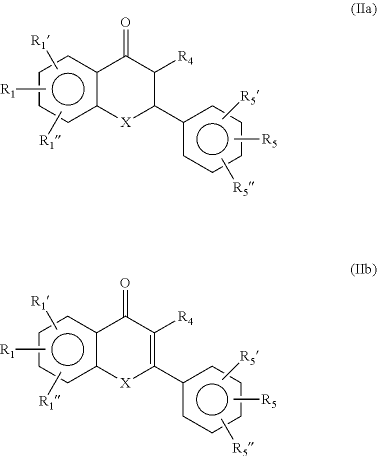

wherein
each of said R₁, R₁′ and R₁″ is independently selected from the group consisting of —H, -halo, —CF₃, —OCF₃, and —OC₁₋₃alkyl;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, —OC₁₋₃alkyl, —C₁₋₃alkyl-OH and —OC₁₋₃alkyl-OH;

The spread of cancer from its tissue of origin and its subsequent growth in other organs is the most life-threatening aspect of the disease. This process is called metastasis, and requires cancer cells to survive and proliferate outside their tissue of origin. The first crucial step in this process is the invasion of cancer cells into tissue surrounding the tumor and the vasculature.

The compounds of the present invention are capable to inhibit said first step and thereby prevent and/or reduce cancer cell dissemination and metastasis formation. As such, the invention relates to the compounds of formula Ila and IIb described herein for use in treating cancer by inhibiting the invasion of tumor cells into surrounding or adjacent tissue, cells and/or their entry into the circulatory system. Hence the compounds of the invention are especially useful for inhibiting or stopping tumor spread.

In a specific aspect, the compounds of formula IIa and IIb of the invention are used as an anti-invasive compound. “Anti-invasive” refers to the ability to inhibit invasion of the tumor or cancer cell into surrounding tissue (within or outside the organ of origin). For determining whether a compound exhibits an anti-invasive behavior the chick heart invasion assay as provided herein or as described in Bracke et al., 2014, may be applied. This screening method confronts cancer cells with a fragment of normal tissue, so as not to neglect the contribution of the host tissue in the micro-ecosystem that governs tumor behavior. The interaction between the cancer cells and the normal tissue is evaluated histologically and classified along a 5-grades subjective scale. Grades III and IV are typical for invasion, while grades 0, I and II correspond to absence of invasion. Compounds that inhibit invasion of confronting cancer cells i.e. from III/IV to 0/I/II, are determined as anti-invasive. Possible alternative assays generally known in the art are the type I collagen invasion assay and the Matrigel invasion assay.

### EXAMPLES

### Materials and Methods

### 1 Synthetic Procedures and Physicochemical Properties

### Reaction Procedures for the Synthesis of Chalcones

### General Protocol: Synthesis of Chalcones

A solution of the appropriate acetophenone (10 mmol) and LiOH·H₂O (10 mol %) in 10 mL of absolute ethanol is stirred at the appropriate temperature for 10 min (for reactions at 40° C., the bulb is equipped with a reflux condenser). Then, the appropriate benzaldehyde (10 mmol, 1 equiv.) is added, and the system is protected from the atmosphere with a cork stopper. Reaction progress is monitored (LCMS). During the reaction, the chalcone may precipitate. Upon attaining maximum conversion grade, the reaction mixture is quenched with 15 mL of 1% hydrochloric acid.

If the chalcone has precipitated, it is isolated by means of filtration. To remove residual amounts of benzaldehyde, the residue is washed thoroughly with water until the filtrate turns clear. The chalcone can be crystallized in absolute ethanol to obtain high-purity crystals.

If the chalcone has formed a separate oily liquor at the bottom of the bulb, it can be extracted from the mixture with diethyl ether. The organic phase is subsequently washed with brine (2×) and dried over MgSO₄, upon which the solution is concentrated in vacuo. Purification of the thus obtained residue can be performed through crystallization in absolute ethanol.

### 3-(4-Fluorophenyl)-1-(3,4,5-trimethoxyphenyl)prop2-en-1-one A1

This substance was prepared according to the general protocol described above.

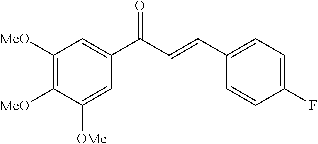 [SMILES: COc1cc(C(=O)/C=C/c2ccc(F)cc2)cc(OC)c1OC]

¹H NMR (CDCl₃, 300 MHz, ppm): δ=3.94 (s, 3H), 3.95 (s, 6H), 7.11 (dd, 1H, J=8.5 Hz, J=8.5 Hz), 7.28 (s, 2H), 7.42 (d, 1H, J=15.4 Hz), 7.64 (dd, 2H, J=8.5 Hz, J=5.2 Hz), 7.78 (d, 1H, J=15.4 Hz); ¹³C NMR (CDCl₃, 75 MHz, ppm): δ=56.3, 61.0, 106.0, 116.1 (d, J=21.9 Hz), 121.3, 130.4 (d, J=8.1 Hz), 131.2 (d, J=3.5 Hz), 133.4, 142.51, 143.4, 153.2, 164.0 (d, J=251.5 Hz), 188.9; ¹⁹F NMR (CDCl₃, 282 MHz, ppm): δ=(−)109.57-(−)109.35 (m); IR (ATR, cm⁻¹): v=1124, 1157, 1227, 1338, 1504, 1580, 1599, 1611, 1660 (C═O); MS (ES+): m/z (%): 317.1 ([M+H]⁺, 100), 318.1 (18), 339.1 (8), 655.2 (8); MP (° C.): 124-125. Yield: 69%, off-white needles (from EtOH); MW=316.32.

### 3-(4-Chlorophenyl)-1-(4-methoxyphenyl)prop2-en-1-one A2

This substance was prepared according to the general protocol described above.

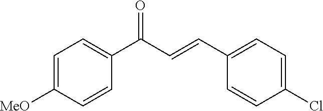 [SMILES: COc1ccc(C(=O)/C=C/c2ccc(Cl)cc2)cc1]

¹H NMR (CDCl₃, 300 MHz, ppm): δ=3.83 (s, 3H), 6.89 (d, 2H, J=8.7 Hz), 7.30 (d, 2H, J=8.7 Hz), 7.41 (d, 1H, J=16.9 Hz), 7.49 (d, 2H, J=8.1 Hz), 7.69 (d, 1H, J=16.9 Hz), 7.99 (d, 2H, J=8.7 Hz);

¹³C NMR (CDCl₃, 75 MHz, ppm): δ=55.6, 113.9, 122.3, 129.2, 129.4, 130.8, 133.6, 136.0, 142.4, 163.5, 188.4. MP (° C.): 127-130° C. Yield: 85%, white needles (from EtOH); MW=272.73.

### 3-(4-Fluorophenyl)-1-phenvlprop2-en-1-one A3

This substance was prepared according to the general protocol described above.

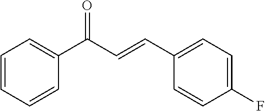 [SMILES: O=C(/C=C/c1ccc(F)cc1)c1ccccc1]

¹H NMR (CDCl₃, 300 MHz, ppm): δ=7.08-7.12 (m, 2H), 7.43-7.52 (m, 3H), 7.56-7.66 (m, 3H), 7.76 (d, 1H, J=15.8 Hz), 7.99-8.01 (m, 2H); ¹³C NMR (CDCl₃, 75 MHz, ppm): δ=116.0 (d, J=21.7 Hz), 121.7, 128.3, 128.5, 130.2 (d, J=8.1 Hz), 131.0 (d, J=3.6 Hz), 132.7, 138.0, 143.4, 164.0 (d, J=251.9 Hz), 190.2. MP (° C.): 86-88° C. Yield: 92%, yellow needles (from EtOH); MW=226.25.

### 1-(4-Fluorophenyl)-3-(3,4,5-trimethoxyphenyl)prop-2-en-1-one A4

This substance was prepared according to the general protocol described above.

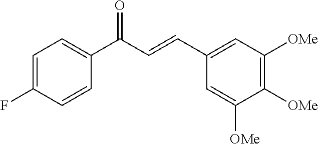 [SMILES: COc1cc(/C=C/C(=O)c2ccc(F)cc2)cc(OC)c1OC]

¹H NMR (CDCl₃, 300 MHz, ppm): δ=3.80 (s, 3H), 3.92 (s, 6H), 7.21 (s, 2H), 7.32 (dd, 2H, J=8.9 Hz, J=8.9 Hz), 7.75 (d, 1H, J=15.7 Hz), 7.85 (d, 1H, J=15.7 Hz), 8.23 (dd, 2H, J=8.9 Hz, J=5.6 Hz); ¹³C NMR (CDCl₃, 75 MHz, ppm): δ=56.7, 60.8, 107.3, 116.5 (d, J=21.9 Hz), 121.8, 131.5, 132.3 (d, J=9.2 Hz), 135.9 (J=2.7 Hz), 141.7, 145.7, 154.7, 166.5 (d, J=252.3 Hz), 188.5. MP (° C.): 102-104. Yield: 85%, white needles (from EtOH); MW=316.32.

### 3-(4-Fluorophenyl)-1-(4-methoxyphenyl)prop2-en-1-one A5

This substance was prepared according to the general protocol described above.

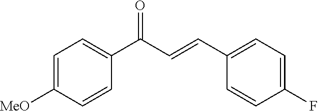 [SMILES: COc1ccc(C(=O)/C=C/c2ccc(F)cc2)cc1]

¹H NMR (CDCl₃, 300 MHz, ppm): δ=3.96 (s, 3H), 6.97 (d, 2H, J=8.5 Hz), 7.11 (dd, 2H, J=8.5 Hz, J=8.5 Hz), 7.49 (d, 1H, J=15.7 Hz), 7.64 (dd, 2H, J=8.5 Hz, J=5.2 Hz), 7.72 (d, 1H, J=15.7 Hz), 8.03 (d, 2H, J=8.5 Hz); ¹³C NMR (CDCl₃, 75 MHz, ppm): δ=56.0, 114.5, 116.3 (d, J=21.9 Hz), 122.4, 130.9, 131.4, 131.5 (d, J=8.5 Hz), 132.0 (d, J=3.5 Hz), 142.3, 163.7, 163.8 (d, J=248.7 Hz), 187.8. MP (° C.): 126-127° C. Yield: 91%, white needles (from EtOH); MW=256.28.

### 3-(Phenyl)-1-(4-methoxyphenyl)prop2-en-1-one A6

This substance was prepared according to the general protocol described above.

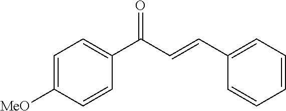 [SMILES: COc1ccc(C(=O)/C=C/c2ccccc2)cc1]

¹H NMR (CDCl₃, 300 MHz, ppm): δ=3.86 (s, 3H), 6.98 (d, 2H, J=8.5 Hz), 7.33-7.43 (m, 3H), 7.52 (d, 1H, J=15.8 Hz), 7.63 (dd, 2H, J=7.4 Hz, J=3.5 Hz), 7.81 (d, 1H, J=15.8 Hz), 8.04 (d, 2H, J=8.5 Hz); ¹³C NMR (CDCl₃, 75 MHz, ppm): δ=55.3, 113.7, 121.8, 128.1, 128.7, 130.1, 130.6, 130.9, 134.9, 143.7, 163.3, 188.5. Yield: 88%, white needles (from EtOH); MW=238.29.

### 3-Phenyl-1-(3,4,5-trimethoxyphenyl)prop2-en-1-one A7

This substance was prepared according to the general protocol described above.

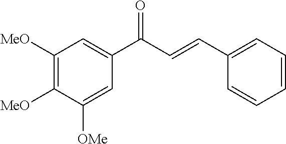 [SMILES: COc1cc(C(=O)/C=C/c2ccccc2)cc(OC)c1OC]

¹H NMR (CDCl₃, 300 MHz, ppm): δ=3.93 (s, 3H), 3.94 (s, 6H), 7.28 (s, 2H), 7.36-7.50 (m, 3H), 7.48 (d, 1H, J=15.8 Hz), 7.58-7.72 (m, 2H), 7.81 (d, 1H, J=15.8 Hz); ¹³C NMR (CDCl₃, 75 MHz, ppm): 8=56.4, 61.0, 106.1, 121.8, 128.4, 129.0, 130.6, 133.5, 134.9, 142.5, 144.9, 153.2, 189.2. Yield: 89%, yellow needles (from EtOH); MW=298.34.

### 1,3-Diphenvlprop2-en-1-one A8

This substance was prepared according to the general protocol described above.

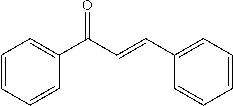 [SMILES: O=C(/C=C/c1ccccc1)c1ccccc1]

¹H NMR (CDCl₃, 300 MHz, ppm): δ=7.39-7.46 (m, 3H), 7.46-7.62 (m, 4H), 7.62-7.69 (m, 2H), 7.76-7.89 (m, 1H), 8.00-8.05 (m, 2H); ¹³C NMR (CDCl₃, 75 MHz, ppm): δ=122.3, 128.7, 128.8, 129.1, 130.8, 133.0, 135.1, 138.4, 145.0, 190.7. Yield: 92%, white needles (from EtOH); MW=224.30.

3-(4-Fluorophenyl)-1-(4-methoxy-3-(trifluoromethyl)phenyl)prop-2-en-1-one A14

This substance was prepared according to the general protocol described above.

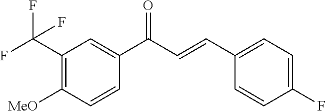 [SMILES: COc1ccc(C(=O)/C=C/c2ccc(F)cc2)cc1C(F)(F)F]

¹H NMR (CDCl₃, 300 MHz, ppm): d=4.01 (s, 3H), 7.11-7.15 (m, 3H), 7.45 (d, 1H), 7.66 (dd, 2H), 7.81 (d, 1H), 8.18-8.33 (m, 2H). Yield: 53%, white needles (from EtOH); MW=324.27.

### Synthesis of Chalcone A9

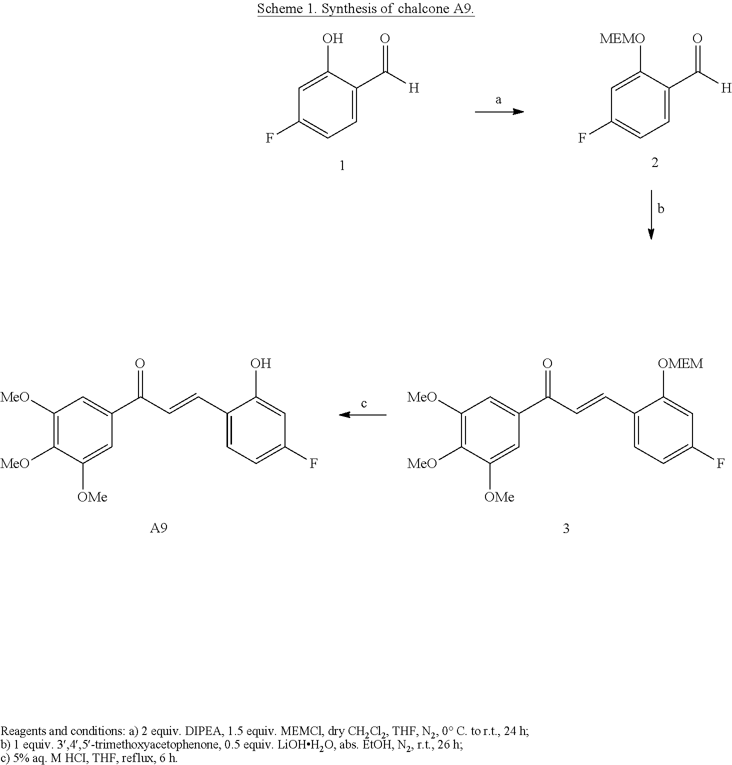

The synthesis of chalcone A9 is depicted in Scheme 1. A flame-dried, round-bottom flask, equipped with a stirring bar and kept under a nitrogen atmosphere, is loaded with 2-hydroxy-4-fluorobenzaldehyde 1 (4.59 mmol), dry CH₂Cl₂ (45 mL) and dry THF (3 mL). The benzaldehyde does not dissolve in CH₂Cl₂, but in THF. The solution is cooled in an ice bath to 0° C. after which 2 equiv. of DIPEA (9.18 mmol) and 1.5 equiv. MEMCl are added. The ice bath is removed and the mixture is stirred at room temperature for 24 h. After completion of the reaction, the mixture is quenched by addition of aq. qat. NaHCO₃ (3 portions of 6 mL). The organics are extracted with EtOAc (2×6 mL) after which the combined organic phases are washed with 1N NaOH (20 mL) and water (20 mL). The organic phase is dried over MgSO₄. If LCMS analysis points towards the residual presence of MEMCl, the product is dissolved in a mixture of sat. aq. NaHCO₃ and THF and stirred intensively for a 20 min period under a N₂ atmosphere, and then extracted with EtOAc and dried over MgSO₄. The protected benzaldehyde 2 (yield 52%) is used as such in the next step.

In a round bottom flask, equipped with a stirring bar, 18.18 mmol 3′,4′,5′-trimethoxyacetophenone is dissolved in 20 mL abs. EtOH. After addition 0.5 equiv. (9.09 mmol) LiOH·H₂O, the mixture is stirred for 10 min under a N₂ atmosphere at room temperature. After addition of 1 equiv. of benzaldehyde 2, the mixture is stirred under a N₂ atmosphere at room temperature for 26 h. The mixture is quenched by addition of 30 mL of water, leading to the formation of a precipitate. The precipitate is isolated by filtration and washed with water until the filtrate is clear. Crystallization of the residue in abs. EtOH yields 3 in the form of white-yellow crystals in a yield of 93%.

Next, the protected chalcone 3 (8.73 mmol) is dissolved in a mixture of 5% aq. HCl (20 mL) and THF (40 mL), after which the mixture is refluxed for 6 h. After completion of the deprotection step (LCMS) the mixture is quenched with brine (60 mL) and the organics are extracted with diethyl ether (2×60 mL). The combined organic phases are extracted with aq. 1 M NaOH (120 mL), and the aqueous phase is subsequently neutralized (pH 7) with aq. HCl. During this step, the clear solution turns into a milky fluid. The product is extracted from this fluid with EtOAc (2×60 mL) and the organic phase is dried over MgSO₄. Crystallization in abs. EtOH yields chalcone A9 in a yield of 55%.

### 3-(4-Fluoro-2-hydroxyphenyl)-1-(3,4,5-trimethoxyphenyl)prop-2-en-1-one A9

Compound A9 was synthesized according to the protocol described above.

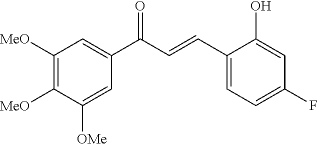 [SMILES: COc1cc(C(=O)/C=C/c2ccc(F)cc2O)cc(OC)c1OC]

¹H NMR (CDCl₃, 300 MHz, ppm): δ=3.95 (s, 6H), 3.95 (s, 3H), 6.67 (ddd, 1H, J=8.5 Hz, J=8.5 Hz, J=2.2 Hz), 6.76 (dd, 1H, J=9.9 Hz, J=2.2 Hz), 7.30 (s, 2H), 7.58 (dd, 1H, J=8.5 Hz, J=6.6 Hz), 7.60 (d, 1H, J=15.4 Hz), 8.21 (d, 1H, J=15.4 Hz), 8.35 (s, 1H); ¹³C NMR (CDCl₃, 75 MHz, ppm): δ=56.4, 61.1, 104.2 (d, J=24.2 Hz), 106.4, 108.0 (d, J=23.1 Hz), 118.7 (d, J=2.3 Hz), 121.2, 130.7 (d, J=10.4 Hz), 133.4, 141.2, 142.6, 153.1, 158.2 (d, J=11.5 Hz), 165.0 (d, J=252.7 Hz), 191.7; ¹⁹F NMR (CDCl₃, 282 MHz, ppm): δ=(−)107.36-(−)107.28 (m); IR (ATR, cm⁻¹) v=717, 801, 853, 976, 1093, 1123, 1161, 1269, 1289, 1314, 1342, 1414, 1495, 1505, 1568, 1643 (C═C), 1656 (C═O), 3268 (OH). MS (ES+): m/z (%)=315.3 (5), 333.3 ([M+H]⁺, 100), 334.0 ([M+H+1]⁺, 17), 396.3 (5), 687.3 (14). MP (° C.)=160. Yield: 55%. brown crystals; MW=332.33.

### General Protocol: Synthesis of Hydroxychalcones

A round-bottom flask, kept under a nitrogen atmosphere, is loaded with 15 mL of 1,2-dimethoxyethane, 2.2 mmol of the appropriate hydroxyacetophenone and 3.5-6 equiv. (0.55 g) of LiOH·H₂O. The resulting suspension is stirred at room temperature for 10 min, whereupon 1.5-3 equiv. of the appropriately substituted benzaldehyde are added. The flask is equipped with a reflux condenser and the mixture is stirred at 60° C. for 48 h. The reaction is quenched via addition of 10 mL of 10% aq. HCl. The organics are extracted with EtOAc (2×15 mL); the organic layers are combined, dried over MgSO₄ and concentrated in vacuo. The resulting crude chalcone is purified via recrystallization in absolute EtOH.

### 3-(4-Fluorophenyl)-1-(6-hydroxy-2,3,4-trimethoxyphenyl)prop-2-en-1-one A10

Compound A10 was synthesized according to the general protocol described above.

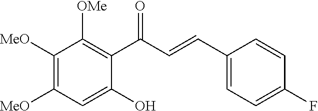 [SMILES: COc1cc(O)c(C(=O)/C=C/c2ccc(F)cc2)c(OC)c1OC]

¹H NMR (CDCl₃, 400 MHz, ppm): δ=3.82 (3H, s), 3.88 (3H, s), 3.91 (3H, s), 6.28 (1H, s), 7.08 (2H, dd, J=8.5 Hz, J=8.5 Hz), 7.60 (2H, dd, J=8.5 Hz, J=5.5 Hz), 7.76 (1H, d, J=15.7 Hz), 7.86 (1H, d, J=15.7 Hz), 13.63 (1H, s). ¹³C NMR (CDCl₃, 100 MHz, ppm): δ=56.2, 61.3, 61.9, 96.7, 108.7, 116.2 (d, J=21.9 Hz), 126.3 (d, J=2.2 Hz), 130.3 (d, J=8.3 Hz), 131.7 (d, J=3.2 Hz), 135.4, 141.9, 155.0, 160.3, 162.8 (3C), 164.0 (d, J=251.3 Hz), 192.8. IR (ATR, cm⁻¹) v=1493, 1507, 1576, 1601, 1610, 1629. MS (ES+): m/z (%)=333.1 ([M+H]⁺, 100), 334.1 ([M+H+1]⁻, 20), 355.0 ([M+Na]⁺, 5). MP (° C.)=103.2. Yield: 76%; bright orange crystals (from EtOH); MW=332.32.

### 3-(4-Fluorophenyl)-1-(2-hydroxy-3,4,5-trimethoxyphenyl)prop-2-en-1-one A11

Compound A11 was synthesized according to the general protocol described above.

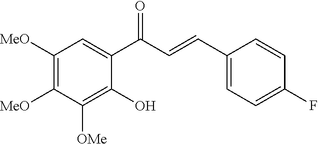 [SMILES: COc1cc(C(=O)/C=C/c2ccc(F)cc2)c(O)c(OC)c1OC]

¹H NMR (CDCl₃, 300 MHz, ppm): δ=3.90 (3H, s), 3.95 (s, 3H), 4.07 (s, 3H), 7.10 (s, 1H), 7.14 (dd, 2H, J=8.7 Hz, J=8.5 Hz), 7.44 (d, 1H, J=15.4 Hz), 7.66 (dd, 2H, J=8.7 Hz, J=5.5 Hz), 7.88 (d, 1H, J=15.4 Hz). ¹³C NMR (CDCl₃, 75 MHz, ppm): δ=57.2, 61.2, 61.5, 107.0, 114.6, 116.4 (d, J=21.9 Hz), 120.1, 130.7 (d, J=8.1 Hz), 131.1 (d, J=2.3 Hz), 141.7, 144.0, 145.2, 150.3, 154.8, 164.4 (d, J=252.7 Hz), 192.4. IR (ATR, cm⁻¹) v=1010, 1039, 1096, 1194, 1270, 1277, 1359, 1407, 1485, 1446, 1505, 1563, 1634. MS (ES+): m/z (%)=333.1 ([M+H]⁺, 100), 333.7 ([M+H+1]⁺, 20), 354.7 ([M+Na]⁺, 9). MP (° C.)=67-68. Yield: 63%; orange-red crystals (from EtOH); MW=332.32.

### 3-(4-fluorophenyl)-1-(2-hydroxy-4-methoxyphenyl)prop-2-en-1-one A12

Compound A12 was synthesized according to the general protocol described above.

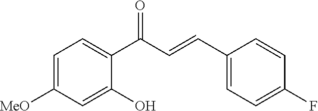 [SMILES: COc1ccc(C(=O)/C=C/c2ccc(F)cc2)c(O)c1]

¹H NMR (CDCl₃, 300 MHz, ppm): δ=3.86 (s, 3H), 6.48 (d, 1H, J=2.2 Hz), 6.49 (dd, 1H, J=9.4 Hz, J=2.2 Hz), 7.12 (dd, 2H, J=8.5 Hz, J=8.5 Hz), 7.50 (d, 1H, J=15.7 Hz), 7.65 (dd, 2H, J=8.5 Hz, J=5.2 Hz), 7.82 (d, 1H, J=9.4 Hz), 7.85 (d, 1H, J=15.7 Hz). ¹³C NMR (CDCl₃, 75 MHz, ppm): 5=55.6, 101.1, 107.9, 114.1, 116.2 (d, J=21.9 Hz), 120.1, 130.5 (d, J=9.2 Hz), 131.1 (d, J=2.3 Hz), 131.3, 143.1, 164.2 (d, J=252.7 Hz), 166.3, 166.8, 191.6. ¹⁹F NMR (CDCl₃, 282 MHz, ppm): δ=(−)109.70-(−)109.00 (m). IR (ATR, cm⁻¹) v=802, 837, 958, 1018, 1137, 1166, 1204, 1218, 1365, 1510, 1568, 1600, 1622, 1633. MS (ES+): m/z (%)=273.3 ([M+H]⁺, 100), 274.3 ([M+H+1]⁺, 16), 308.3 (25). MP (° C.)=135. Yield: 71%, pale-yellow crystals (from EtOH); MW=272.27.

### 3-(4-chlorophenyl)-1-(2-hydroxy-4-methoxyphenyl)prop-2-en-1-one A13

Compound A13 was synthesized according to the general protocol described above.

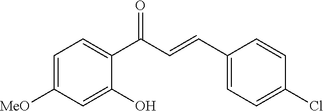 [SMILES: COc1ccc(C(=O)/C=C/c2ccc(Cl)cc2)c(O)c1]

1H NMR (CDCl₃, 300 MHz, ppm): δ=3.86 (s, 3H), 4.77 (br. s, 1H), 6.48 (d, 1H, J=2.2 Hz), 6.49 (dd, 1H, J=8.8 Hz, J=2.2 Hz), 7.40 (d, 2H, J=8.8 Hz), 7.54 (d, 1H, J=15.4 Hz), 7.58 (d, 1H, J=8.8 Hz), 7.81 (d, 1H, J=8.8 Hz), 7.82 (d, 1H, J=15.4 Hz). 13C NMR (CDCl₃, 75 MHz, ppm): δ=55.7, 101.2, 108.0, 114.1, 120.9, 129.4, 129.8, 131.3, 133.4, 136.7, 143.0, 166.5, 166.9, 191.6. IR (ATR, cm⁻¹) v=790, 824, 958, 1085, 1122, 1202, 1218, 1273, 1359, 1562, 1577, 1618, 1636, 3364. MS (ES+): m/z (%)=289.1 ([M+H]⁺, 100), 290.0 ([M+1+H]⁺, 19), 291.0 ([M+2+H]⁺, 37), 292.0 ([M+3+H]⁺, 7). HRMS (ES+): m/z=Calculated for C₁₆H₁₃ClO₃+H⁺ [M+H]⁺: 289.0626; found: 289.0624. MP (° C.)=131. Yield: 69%, bright yellow fibers (from EtOH); MW=288.73.

### Reaction Procedures for the Synthesis of Flav(an)ones

### General Protocol: Oxidative Cyclization of 2′-Hydroxychalcones Towards Flavones

To a round-bottom flask containing 5 mL of DMSO, 0.63 mmol of the appropriate 2′-hydroxychalcone and 4-11 mol % of I₂ are added. The resulting solution is heated to reflux temperature for 2-5 h, and then poured onto 10 mL of an ice-water mixture. The resulting blue residue is isolated by filtration and redissolved in 10 mL EtOAc. The thus obtained solution is washed with 10 mL 10% aq. Na₂S₂O₃, dried over MgSO₄ and evaporated till dryness, furnishing the crude flavone as a pure solid.

### 2-(4-Fluorophenyl)-5,6,7-trimethoxy-4H-chromen-4-one B1

Compound B1 was synthesized according to the general protocol for oxidative cyclization described above.

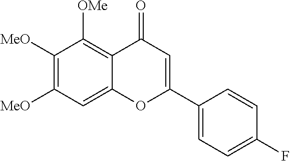 [SMILES: COc1cc2oc(-c3ccc(F)cc3)cc(=O)c2c(OC)c1OC]

¹H NMR (CDCl₃, 300 MHz, ppm): δ=3.92 (3H, s), 3.99 (3H, s, 3.99 (3H, s), 6.61 (s, 1H), 6.80 (s, 1H), 7.20 (2H, dd, J=8.8 Hz, J=8.8 Hz), 7.88 (2H, dd, J=8.8 Hz, J=5.2 Hz). ¹³C NMR (CDCl₃, 75 MHz, ppm): δ=56.4, 61.7, 62.3, 96.3, 108.3, 113.0, 116.3 (d, J=21.9 Hz), 127.9 (d, J=3.5 Hz), 128.3 (d, J=8.1 Hz), 140.6, 152.7, 154.6, 157.9, 160.3, 164.7 (d, J=252.7 Hz), 177.2. IR (ATR, cm-1) v=1120, 1346, 1420, 1486, 1513, 1605, 1653. MS (ES+): m/z (%)=331.1 ([M+H]⁺, 100), 332.1 ([M+H+1]⁺, 19). HRMS (ES+): m/z=Calculated for C₁₈H₁₅FO₅+H+[M+H]⁺: 331.0976; Found: 331.0974. MP (° C.)=172-173. Yield: 98%; beige powder; MW=330.31.

### 2-(4-Fluorophenyl)-6,7,8-trimethoxy-4H-chromen-4-one B2

Compound B2 was synthesized according to the general protocol for oxidative cyclization described above.

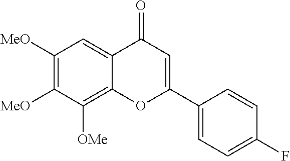 [SMILES: COc1cc2c(=O)cc(-c3ccc(F)cc3)oc2c(OC)c1OC]

¹H NMR (CDCl₃, 300 MHz, ppm): δ=3.97 (s, 3H), 4.05 (s, 3H), 4.09 (s, 3H), 6.75 (s, 1H), 7.23 (dd, 2H, J=9.1 Hz, J=9.1 Hz), 7.39 (s, 1H), 7.95 (dd, 2H, J=9.1 Hz, J=5.2 Hz). ¹³C NMR (CDCl₃, 75 MHz, ppm): δ=56.31, 61.50, 62.08, 100.02, 106.59, 116.38 (d, J=21.9 Hz), 119.76, 128.15 (d, J=3.5 Hz), 128.34 (d, J=9.2 Hz), 142.12, 145.78, 147.54, 151.32, 161.90, 164.73 (d, J=253.8 Hz), 177.63. IR (ATR, cm⁻¹) v=1075, 1108, 1125, 1366, 1378, 1414, 1466 (C═C), 1650 (C═O). MS (ES+): m/z (%)=330.7 ([M+H]⁺, 100), 331.8 ([M+H+1]⁺, 19). Yield: 70%; off-white fibers (from EtOH); MW=330.31.

### 2-(4-Fluorophenyl)-7-methoxy-4H-chromen-4-one B3

Compound B3 was synthesized according to the general protocol for oxidative cyclization described above. The product was crystallized in EtOH to increase the degree of purity.

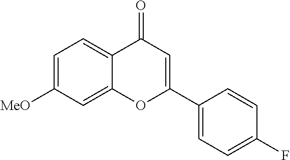 [SMILES: COc1ccc2c(=O)cc(-c3ccc(F)cc3)oc2c1]

¹H NMR (CDCl₃, 300 MHz, ppm): δ=3.93 (s, 3H), 6.70 (s, 1H), 6.96 (d, 1H, J=2.2 Hz), 6.99 (dd, 1H, J=8.8 Hz, J=2.2 Hz), 7.21 (dd, 2H, J=8.8 Hz, J=8.5 Hz), 7.92 (dd, 2H, J=8.8 Hz, J=5.2 Hz), 8.13 (d, 1H, J=8.8 Hz). ¹³C NMR (CDCl₃, 75 MHz, ppm): δ=55.9, 100.4, 107.2, 114.5, 116.2 (d, J=23.1 Hz), 117.7, 127.0, 128.0 (d, J=3.5 Hz), 128.3 (d, J=9.2 Hz), 157.9, 161.9, 164.2, 164.6 (d, J=253.8 Hz), 177.6. ¹⁹F NMR (CDCl₃, 282 MHz, ppm): δ=(−)108.37-(−)108.27 (m). IR (ATR, cm⁻¹) v=810, 827, 838, 1084, 1164, 1202, 1233, 1351, 1372, 1416, 1437, 1509, 1586, 1606, 1628, 1636. MS (ES+): m/z (%)=271.3 ([M+H]⁺, 100), 272.0 ([M+H+1]⁺, 15). MP (° C.)=163. Yield: 74%, off-white crystals (from EtOH); MW=270.26.

### 2-(4-Chlorophenyl)-7-methoxy-4H-chromen-4-one B4

Compound B4 was synthesized according to the general protocol for oxidative cyclization described above.

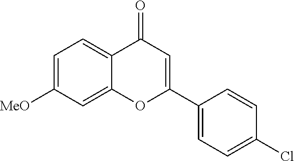 [SMILES: COc1ccc2c(=O)cc(-c3ccc(Cl)cc3)oc2c1]

¹H NMR (CDCl₃, 300 MHz, ppm): δ=3.94 (s, 3H), 6.73 (s, 1H), 6.96 (d, 1H, J=2.2 Hz), 7.00 (dd, 1H, J=8.8 Hz, J=2.2 Hz), 7.50 (d, 2H, J=8.8 Hz), 7.85 (d, 2H, J=8.8 Hz), 8.13 (d, 1H, J=8.8 Hz). MS (ES+): m/z (%)=271.3 ([M+H]⁺, 100), 272.0 ([M+H+1]⁺, 15). Yield: 97%, beige powder; MW=286.71.

### General Protocol: Cyclization of 2′-Hydroxychalcones Towards Flavanones Under Acidic Conditions

To a round-bottom flask containing 7 mL of 3 N HCl in MeOH, 0.27 mmol of the appropriate 2′-hydroxychalcone is added. The resulting solution is heated to reflux temperature for 24 h, and then concentrated in vacuo. Then, H₂O (7 mL) is added, and the resulting suspension is neutralized with saturated aq. NaHCO₃ (7 mL). Next, an extraction with EtOAc (15 mL) is performed. The thus obtained organic layer is washed with H₂O (10 mL), dried over MgSO₄ and evaporated till dryness, furnishing the crude flavanone as a solid. Subsequent recrystallization in absolute EtOH furnishes the pure product.

### 2-(4-Fluorophenyl)-5,6,7-dimethoxychroman-4-one B5

Compound B5 was synthesized according to the general protocol for cyclization under acidic conditions described above.

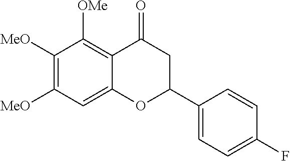 [SMILES: COc1cc2c(c(OC)c1OC)C(=O)CC(c1ccc(F)cc1)O2]

¹H NMR (CDCl₃, 400 MHz, ppm): δ=2.77 (dd, 1H, J=16.7 Hz, J=2.8 Hz), 2.98 (dd, 1H, J=16.7 Hz, J=13.3 Hz), 3.82 (s, 3H), 3.88 (s, 3H), 3.94 (s, 3H), 5.38 (dd, 1H, J=13.3 Hz, J=2.8 Hz), 6.34 (s, 1H), 7.11 (t, 2H, J=8.7 Hz), 7.43 (dd, 2H, J=8.7 Hz, J=5.3 Hz). ¹³C NMR (CDCl₃, 100 MHz, ppm): δ=45.7, 56.3, 61.5, 61.7, 78.8, 96.4, 109.3, 115.9 (d, J=21.9 Hz), 128.1 (d, J=8.1 Hz), 134.7 (d, J=2.9 Hz), 137.8, 154.4, 159.54 and 159.57 (3C), 162.9 (d, J=247.6 Hz), 189.1. IR (ATR, cm⁻¹) v=1083, 1103, 1264 (C—O), 1412, 1461, 1483, 1514 (C═C), 1599 (C═O). MS (ES+): m/z (%)=333.1 ([M+H]⁺, 100), 334.1 ([M+1+H]⁺, 19). HRMS (ES+): m/z=Calculated for C₁₈H₁₇FO₅+H+[M+H]⁺: 333.1133; found: 333.1134. Yield: 44%; white powder (from EtOH); MW=332.32.

### 2-(4-Fluorophenyl)-6,7,8-dimethoxychroman-4-one B6

Compound B6 was synthesized according to the general protocol for cyclization under acidic conditions described above.

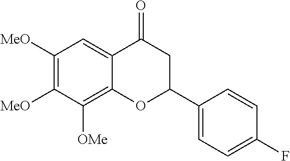 [SMILES: COc1cc2c(c(OC)c1OC)OC(c1ccc(F)cc1)CC2=O]

¹H NMR (CDCl₃, 300 MHz, ppm): δ=2.87 (dd, 1H, J=16.9 Hz, J=3.3 Hz), 3.03 (dd, 1H, J=16.9 Hz, J=12.8 Hz), 3.87 (s, 3H), 3.90 (s, 3H), 4.01 (s, 3H), 5.46 (dd, 1H, J=12.8 Hz, J=3.3 Hz), 7.12 (t, 2H, J=8.7 Hz, J=8.7 Hz), 7.17 (s, 1H), 7.47 (dd, 2H, J=8.7 Hz, J=5.2 Hz). ¹³C NMR (CDCl₃, 75 MHz, ppm): δ=44.6, 56.3, 61.5, 61.6, 79.6, 102.5, 115.9 (d, J=21.9 Hz), 116.0, 128.1 (d, J=9.2 Hz), 134.8 (d, J=3.5 Hz), 142.2, 148.2, 149.7, 151.1 (3C), 162.9 (d, J=246.9 Hz), 190.9. IR (ATR, cm⁻¹) v=1068, 1111, 1157, 1219, 1283, 1354, 1418, 1455, 1470 (C═C), 1681 (C═O). MS (ES+): m/z (%)=332.8 ([M+H]⁺, 100), 333.7 ([M+1+H]⁺, 20), 354.7 ([M+Na]⁺, 20), 395.7 (5). Yield: 50%; pale yellow solid (from EtOH); MW=332.32.

### General Protocol: Cyclization of 2′-Hydroxychalcones Towards Flavanones Under Basic Conditions

A flame-dried round-bottom flask equipped with a reflux condenser and a CaCl₂-tube is charged with 10 mL of absolute EtOH. The solvent is heated to reflux after which the appropriate 2′-hydroxychalcone (1 mmol) and 10 equiv. of NaOAc are added. Reflux is maintained and the reaction is monitored via LC/MS. When maximum conversion has been obtained, the yellow reaction mixture is allowed to cool to room temperature and poured into 10 mL of ice water. The organics are extracted with EtOAc (3×20 mL) and the combined organic layers are dried washed with brine, dried over MgSO4 and concentrated in vacuo. A yellow residue is obtained containing the containing a mixture of 2′hydroxychalcone and flavanone. Separation is performed using preparative TLC using an eluent mixture of petroleum ether and EtOAc (8:1). The flavanone is thus obtained as a beige solid.

### 2-(4-Fluorophenvl)-7-methoxvchroman-4-one B7

Compound B7 was synthesized according to the general protocol for cyclization under basic conditions described above.

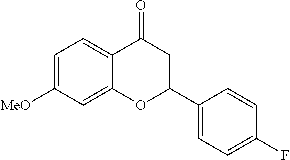 [SMILES: COc1ccc2c(c1)OC(c1ccc(F)cc1)CC2=O]

¹H NMR (CDCl₃, 300 MHz, ppm): δ=2.81 (dd, 1H, J=16.7 Hz, J=2.8 Hz), 3.02 (dd, 1H, J=16.7 Hz, J=12.9 Hz), 3.84 (s, 3H), 5.46 (dd, 1H, J=12.9 Hz, J=2.8 Hz), 6.49 (d, 1H, J=2.2 Hz), 6.63 (dd, 1H, J=8.8 Hz, J=2.2 Hz), 7.12 (dd, 2H, J=8.5 Hz, J=8.5 Hz), 7.46 (dd, 2H, J=8.4 Hz, J=5.2 Hz), 7.87 (d, 1H, J=8.8 Hz). ¹³C NMR (CDCl₃, 75 MHz, ppm): δ=44.4, 55.7, 79.4, 101.0, 110.4, 114.8, 115.8 (d, J=21.9 Hz), 128.1 (d, J=8.1 Hz), 128.8, 134.8 (d, J=2.3 Hz), 162.8 (d, J=248.1 Hz), 163.4, 166.3, 190.3. ¹⁹F NMR (CDCl₃, 282 MHz, ppm): δ=(−)113.39-(−)113.30 (m). IR (ATR, cm⁻¹) v=804, 820, 830, 841, 1112, 1160, 1200, 1220, 1232, 1256, 1442, 1514, 1572, 1602, 1671. MS (ES+): m/z (%)=273.3 ([M+H]⁺, 100), 274.3 ([M+H+1]⁺, 29). MP (° C.)=93. Yield: 66%, beige solid; Chromatography: Rf: 0.15 (petroleum ether/EtOAc 8:1); MW=272.27.

### 2-(4-Chlorophenyl)-7-methoxvchroman-4-one B8

Compound B8 was synthesized according to the general protocol for cyclization under basic conditions described above.

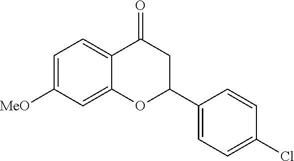 [SMILES: COc1ccc2c(c1)OC(c1ccc(Cl)cc1)CC2=O]

¹H NMR (CDCl₃, 300 MHz, ppm): δ=2.82 (dd, 1H, J=16.7 Hz, J=3.0 Hz), 2.99 (dd, 1H, J=16.7 Hz, J=13.1 Hz), 3.85 (s, 3H), 5.45 (dd, 1H, J=13.1 Hz, J=3.0 Hz), 6.50 (d, 1H, J=2.2 Hz), 6.63 (dd, 1H, J=8.8 Hz, J=2.2 Hz), 7.41 (s, 4H′), 7.87 (d, 1H, J=8.8 Hz). IR (ATR, cm⁻¹) v=811, 825, 832, 1015, 1062, 1116, 1226, 1446, 1574, 1615, 1674. MS (ES+): m/z (%)=289.0 ([M+H]⁺, 100), 290.0 ([M+H+1]⁺, 16), 291.1 ([M+H+2]⁺, 36), 292.0 ([M+H+3]⁺, 7). Yield: 52%, beige solid; Chromatography: Rf: 0.18 (petroleum ether/EtOAc 8:1); MW=288.73.

### 2 Cell Cultures

The Michigan Cancer Foundation-7 (MCF-7) cell line is a human breast cancer cell line originally isolated from a pleural effusion of a breast adenocarcinoma. This cell line was further selected for invasiveness and metastatic activity, which resulted in the MCF-7/6 cell line, donated by H. Rochefort (Unité d'Endocrinologie Cellulaire et Moléculaire, Montpellier, France). Human 13B20332 hTERT (also referred to as 13B2 hTERT) breast CAFs were derived from a tumor that was 99% estrogen receptor-positive, 25% progesterone receptor-positive and HER2-negative, and were isolated as previously described (Primac et al. 2019). IF staining and flow cytometry of 13B2 hTERT CAF cells showed positive staining for α-SMA, FAP, VIM, CD44, CD105 and CD90 and negative expression of epithelial marker CD326. Human CT5.3 colon CAFs were isolated from a colorectal adenocarcinoma resection specimen obtained in accordance with the local ethics committee (Ghent University Hospital) and infected with a pBABE retroviral vector expressing the hTERT open reading frame (De Vlieghere et al. 2015). IF staining and flow cytometry of CT5.3 hTERT CAF cells showed positive staining for α-SMA, FAP, VIM, CD44, CD105 and CD90 and negative expression of epithelial marker CD326. HCA2 hTERT dermal fibroblasts were donated by C. Jones (Cardiff University, UK). 4T1-luc mammary tumor cells were a kind gift from Prof. Clare Isacke (Breakthrough Breast Cancer Research Centre, London, UK). This aggressive cell line resembles human metastatic triple negative breast cancer (TNBC) and constitutively expresses firefly luciferase. Primary human atrial fibroblasts were obtained from right atrial appendage tissue of patients in sinus rhythm undergoing open heart surgery and isolated using the tissue explant method. The epicardial layer was removed and pieces of 1-2 mm² were cultured in plates with abraded surfaces using 2 mL Dulbecco's Modified Eagle Medium supplemented with 10% fetal bovine serum and 1% penicillin/streptomycin (all Sigma-Aldrich, Germany). Resulting outgrowing fibroblasts were considered passage zero. Primary glioblastoma cells were obtained using a similar protocol from patients undergoing glioblastoma resection surgery. All patients gave informed consent on the use of the tissue. CAF03 glioblastoma CAFs were purchased from Vitro Biopharma (US).

MCF-7/6 cells were cultured in cultured in medium containing Dulbecco's modified Eagle medium (DMEM) (cat. no. 41965039, Thermo Fisher Scientific) and F12 Nut mix (Ham) medium (cat. no. 11765054, Thermo Fisher Scientific) in a 1:1 ratio supplemented with 10% heat-inactivated fetal bovine serum (FBS) (cat. no. ATCC-30-2030, LGC Standards), 100 IU ml-1 penicillin and 100 mg ml-1 streptomycin (cat. no. 15070063, Thermo Fisher Scientific). CT5.3 hTERT, HCA2 hTERT and 4T1-luc cells were cultured in DMEM (cat. no. 41965039, Thermo Fisher Scientific) supplemented with 10% heat-inactivated fetal bovine serum (FBS) (cat. no. ATCC-30-2030, LGC Standards), 100 IU ml-1 penicillin and 100 mg ml-1 streptomycin (cat. no. 15070063, Thermo Fisher Scientific). The 13B2 hTERT cell line were cultured in the same medium as described above with the following additives: 0.05 μg/mL choleratoxin (cat. no. C8052, Sigma-Aldrich), 5% human serum (cat. no. P2918, Sigma-Aldrich), 0.01 μM triiodothyronine (cat. no. IRM6649, Sigma-Aldrich), 0.01 μM hydrocortisone (cat. no. H0888, Sigma-Aldrich), 0.01 μg/mL epidermal growth factor (EGF) (cat. no. E9644, Sigma-Aldrich) and 0.05 mg/mL insulin (cat. no. 16634, Sigma-Aldrich). Cells were expanded and maintained as a monolayer at 37° C. in an atmosphere of 5% CO2 (MCF-7/6, 4T1-luc) or 10% CO2 (CT5.3 hTERT, HCA2 hTERT, 13B2 hTERT) in air and passaged at 80% confluence. All human cell lines were tested monthly using the Mycoalert Mycoplasma Detection Kit (cat. no. LT07-318, Lonza) to exclude mycoplasma contamination. Primary human atrial fibroblasts were cultured in DMEM supplemented with 2 mM L-alanyl-L-glutamine (GlutaMAX, cat. no. 31966021, LifeTechnologies), 10% fetal calf serum (cat. no. F9665, Sigma-Aldrich) and 1% penicillin/streptomycin (cat. no. 15070063, Thermo Fisher Scientific). CAF03 glioblastoma CAFs and primary glioblastoma cells were grown in MSC-GRO™ VitroPlus III Low Serum Complete Medium (Vitro Biopharma, US).

### 3 Chick Heart Invasion Assay

Chick heart invasion assays were performed as described earlier (Bracke et al. 2014). Precultured heart fragments of 9-day-old chicken embryos were confronted with MCF-7/6 aggregates on top of a semi-solid agar at 37° C. overnight, after which the confronting pairs were cultured in suspension for 8 days at 37° C., 10% CO₂ in presence or absence of a test compound (in duplo). This procedure can also be performed with aggregates of 4T1-Luc cells, for which the suspension culture of the confronting pairs is performed for 3.5 days. All compounds were evaluated at 1 μM and 100 nM; depending on the outcome higher or lower concentrations were subsequently evaluated. Upon fixation in Bouin-Hollande, the cultures were dehydrated and embedded in paraffin. Sections of the confrontation cultures were stained with hematoxylin-eosin are blinded and scored by a trained person (Bracke et al., 2014).

In the chick heart invasion assay cancer cells are confronted with a fragment of normal tissue, so as not to neglect the contribution of the host tissue in the micro-ecosystem that governs tumor behavior. As such, precultured heart tissue fragments (PHFs), dissected from 9-day-old chicken embryos, may be confronted with aggregates of human invasive MCF-7/6 mammary carcinoma cells or other cells in the presence of a certain concentration of a test compound. After eight days for MCF-7/6, or another period depending on the cell type, the interaction between the cancer cells and the PHF is evaluated histologically and classified along a 5-grades subjective scale (Bracke et al. 2014). Grades III and IV are typical for invasion, while grades 0, I and II correspond to absence of invasion. Compounds that inhibit invasion of confronting cancer cells i.e. from III/IV to 0/I/II, are determined as anti-invasive. Possible alternative assays generally known in the art are the type I collagen invasion assay, wound healing assay and the matrigel invasion assay.

### 4 Atomic Force Microscopy

U-shaped, 384-well ULA plates (cat. no. MS-9384UZ, S-bio) were seeded with a suspension of cell-specific culture media with cells. Cells were allowed to form spheroids for an appropriate time under treatment conditions before use in experiments. Cell culture dishes (cat. no. 734-2814, VWR) were filled with collagen gel and placed at 37° C. for an appropriate time to allow solidification. For each condition, spheroids were pooled in one cell culture dish. The medium was removed (stored at 37° C.) to promote attachment of the bottom of each spheroid to the collagen gel. The cell culture dishes were incubated at 37° C. After the incubation period, the stored medium was carefully (prevent detachment of spheroids) added to the dishes.

Mechanical measurements were performed using an atomic force microscope. To indent the spheroids an in-house made colloidal probe was used. Force curves were collected using an appropriate setpoint and probe speed (depending on the stiffness of the spheroids). On each spheroid multiple force curves were collected. Collected curves were then processed using the JPK DP software. Young's modulus was extracted from the force curves. All experiments were performed in biological triplicate.

### 5 Stiffness Measurements

CAFs are a dominant cell type in the TME and are implicated in all stages in cancer progression. CAFs are generally larger and more metabolically active than healthy fibroblasts. They promote angiogenesis and immune evasion by producing an array of growth factors and cytokines, such as transforming growth factor-β (TGF-β), vascular endothelial growth factor (VEGF), interleukin-6 (IL-6) and CXC-chemokine ligand (CXCL12). Another important characteristic of CAFs is their capacity to remodel the ECM, which ultimately contributes to metastasis. CAFs are the main producers of ECM constituents and thus contribute extensively to its remodeling, including the stiffness of the ECM.

To generate 3D cultures mimicking the complexity and pathophysiology of in vivo tumors, spheroids were used (cfr. point 4). Spheroids are considered to better simulate cellular heterogeneity, nutrient and oxygen gradients, cell-cell interactions, cell-matrix interactions, gene expression profiles, stromal interactions, therapeutic responses, multicellular resistance, drug penetration and anti-apoptotic signaling.

### 6 Evaluation of Cell Viability.

The half maximal inhibitory concentration (IC₅₀) of A1 was evaluated using a CellTiter-Glo® 2.0 Cell Viability Assay (cat. no. G9243, Promega) and a CellTiter-Glo® 3D Cell Viability Assay (cat. no. G9681, Promega). 13B2 hTERT cells (2D: 2×10⁵, 3D: 8×10⁴) were plated in each well of a Nunc™ MicroWell™ 96-Well, Nunclon Delta-Treated, Flat-Bottom Microplate (cat. no. 167008, Thermo Fisher Scientific) for 2D experiments or 96-well ULA plate (cat. no. MS-9096UZ, S-bio) in a volume of 90 μL/well. A1 was serially diluted in the appropriate medium to 10× the final concentrations. For each treatment condition, 10 μL was added to 3 replicate wells (final concentrations ranging from 1-100 μM) and plates were incubated for 48 h.

The CellTiter-Glo Cell Viability Assay was performed according to manufacturer's instructions. Briefly, the plates and the CellTiter-Glo® Reagent were equilibrated to room temperature (RT). The CellTiter-Glo® Reagent was added to each well in a 1:1 (v/v) ratio. For 2D experiments, cell lysis was induced by placing the plates on an orbital shaker for 2 min, followed by stabilization of the luminescent signal for 10 min. For 3D experiments, the plates were placed on the orbital shaker for 5 minutes, followed by stabilization for 25 min. Afterwards, the luminescence was measured using a Synergy™ HTX Multi-Mode Microplate Reader (BioTek). The IC₅₀ value was determined using the Graphpad Prism software. This was performed for 3 biological replications.

### 7 In Vivo Experiment: 4T1-Based Intraductal Model

### 7.1 Intraductal Inoculation of 4T1-Luc Cells

Experiments in mice were performed according to Good Scientific Practice-principles and approved by the Ethical Committee (EC) of the Faculty of Veterinary Medicine, Ghent University (EC 2019-105).

Intraductal inoculations were performed in the third mammary gland pair of lactating female mice. In order to become lactating, 8-w-old female BALB/c mice were mated with male BALB/c mice of the same age and pups were weaned 12-14 d after delivery. One hour after weaning, lactating females received inhalation anesthesia using a mixture of oxygen and isoflurane (2-3%) and were subsequently inoculated through the mammary teat canal using a 32-gauge blunt needle with 5×10⁴ 4T1-luc cells suspended in a 100 μl mixture of PBS and Matrigel® (1:10; Corning, Bedford, MA, USA). All necessary materials for inoculation were pre-cooled to avoid Matrigel® coagulation. Analgesia was provided through i.p. administration of buprenorphine (10 μg/kg, Val d'Hony Verdifarm NV-Belgium).

### 7.2 Treatment Preparation and Administration

Compounds were dissolved in DMSO. The compound solution was subsequently aspired using a 1 mL syringe and an 18 G (pink) needle, including additional air to help eject all the solution and avoid retention of compound in the dead volume. The solution was injected into a Medigel in five parts at five different sites and a volume of gel was aspired and reinjected into the cup at the last site to rinse the dead volume of the syringe. The medication was shaken vigorously afterwards and visually examined for even dispersion of compound. Mice received freshly prepared cups with medication every 48 h and gels were placed in gel cup holders to avoid contact with bedding material and absorbent shavings in the mouse cage. The amount of Medigel given to the mice was weekly determined and depended on the mean mouse body weight and consumption. The dosing level was kept constant at 100 mg/kg three times per day.

### 7.3 Analysis of Tumor and Metastasis Progression

Body weight and body temperature were measured every day during the first week p.i., and once a week during the following 5 weeks (i.e. up to 6 w p.i.) using a digital weighing scale and a rectal temperature probe, respectively. As a measurement of primary tumor growth, primary tumor volumes were weekly monitored using a digital caliper and 4T1-luc-derived bioluminescence in primary tumors was monitored at 1, 3 and 6 w p.i. using the IVIS lumina III system (PerkinElmer, Zaventem, Belgium). To perform in vivo imaging, mice were injected with 200 μl D-luciferin suspended in PBS (2 mg/100 μl; Gold Biotechnology, St. Louis, MO) and images were acquired 10 min later under inhalation anesthesia. To perform ex vivo imaging, mice were sedated with a mixture of 100 mg/kg ketamine (Ketamidor, Ecuphar nv/sa, Oostkamp, Belgium) and 10 mg/kg xylazine (Xylazini Hydrochloridum, Val d'HonyVerdifarm, Beringen, Belgium) and subsequently sacrificed through cervical dislocation. Images of bioluminescent signals in isolated primary tumors, spleens and metastases-bearing organs (including axillary lymph nodes, lungs and liver) were quickly acquired using the IVIS system. Primary tumors and spleens were also weighed using a digital weighing scale. Analysis of the in vivo and ex vivo bioluminescent signals was performed using the living image software 4.7.2.

### 7.4 Histology and Immunohistological Stainings

Isolated tissue of primary tumors, axillary lymph nodes, lungs and livers was fixed in buffered 3.5% formaldehyde for 24 h at room temperature (RT) and embedded in paraffin wax. Sections of 5 μm were deparaffinized, hydrated and stained with hematoxylin and eosin (H&E). To allow analysis, sections were dehydrated and mounted.

For Masson's trichrome staining, 3-5 μm thick sections were deparaffinized, hydrated and incubated ON in Bouin's solution to improve staining quality. The following day, slides were washed in distilled H₂O (dH₂O) and subsequently submerged 3 times in Biebrich scarlet-acid fuchsin solution, washed again in dH₂O and incubated for 20 min in phosphomolybdic-phosphotungstic acid solution. Following the incubation in phosphomolybdic-phosphotungstic acid solution, slides were not rinsed, but immediately incubated in aniline blue solution for 8 min. After a final washing step with dH₂O, slides were incubated in 1% acetic acid solution for 8 min. Sections were then quickly dehydrated through 94% ethyl alcohol, absolute alcohol to remove Biebrich scarlet-acid fuchsin staining and xylene to clear the slide before mounting with Tissue-Tek glass mounting medium. The staining visualizes collagen in blue, nuclei in dark red, cytoplasm in red/pink. Quantification of positive staining was established using color deconvolution (for Masson's trichrome staining) followed by automatic counting in ImageJ.

For immunohistochemical staining, antigen retrieval was performed on 3-5 μm thick deparaffinized sections with citrate buffer (10 mM tri-sodium citrate (Santa Cruz Biotechnology, Heidelberg, Germany) for α-SMA, CD11 b, Ly6G, and CD163), pH 6 or with Tris-EDTA buffer [pH 9, 10 mM Tris, 1 mM EDTA (Thermo Fisher Scientific) for PDPN and FAP-α) at 95° C. for 30 min using a pressurized Decloaking Chamber NxGen (Biocare Medical, CA, USA). The slides were cooled down to RT for 30 min and subsequently incubated on an orbital shaker at 20 rpm in a closed microscope box with tris-buffered saline (TBS, Biocare Medical)-wetted tissue paper for all blocking, rinsing (between every incubation step with TBS, 3 times for 2 min at RT) and staining steps. To block endogenous peroxidase activity, slides were first treated with 3% H₂O₂ in methanol for 10 min. To block non-specific binding sites, the slides were subsequently treated with serum-free protein block (Dako, Heverlee, Belgium) for 10 min.

The slides were then stained with primary antibodies diluted in Antibody Diluent (Dako) for 1 h at RT followed by incubation with secondary antibody for 30 min at RT. The used primary antibodies and dilutions were: anti-α-SMA (1:2000, clone EPR5368, Abcam, Cambridge, UK), anti-PDPN (1:100, clone PMab-1, Abcam), anti-FAP-α (1:100, polyclonal, Abcam), anti-CD11b (1:4000, clone EPR1344, Abcam), anti-Ly6G (1:1000, clone 1A8, Biolegend, CA, USA), anti-CD163 (1:500, clone EPR19518, Abcam). The used secondary antibodies were Rat-on-Mouse HRP-Polymer (Biocare Medical) for PDPN and Ly6G, and ready to-use Dako EnVision+ Rabbit for α-SMA, FAP-α, CD11b, and CD163. For visualization of HRP-positive staining, slides were treated with a 3,3′-diaminobenzidine (DAB)-containing buffer (Dako) for 10 min at RT. Counterstaining with hematoxylin was applied for 5 min at RT, followed by dehydration and mounting of the slides with Tissue-Tek glass mounting medium. Quantification of positive staining was established using color deconvolution (for DAB and hematoxylin counterstaining) followed by automatic counting in ImageJ.

### 7.5 Cytokine Array Analysis

The Proteome Profiler Mouse XL Cytokine Array (cat. no. ARY028, R&D Systems) was performed with syngeneic 4T1 TNBC mouse model primary tumor lysates according to manufacturer's instructions. Analyses were performed using the ImageJ plugin ‘Protein Array Analyzer’.

### 7.6 Statistical Analysis

Data are presented in graphs as the means+/−standard error of the mean (SEM). *, ** and represent statistically significant differences at the level of the values P<0.05, P<0.01, and P<0.001, respectively. Statistics were performed using Prism (Graphpad) and data were normalized using log 10 normalization when necessary. P-values were calculated by one-tailed unpaired t-tests or one-way Analysis of Variance (ANOVA) tests followed by Newman-Keuls post-hoc test for multiple comparisons.

### 8 Combination Treatment

Combined inactivation of E-cadherin and PTEN in mouse mammary epithelium results in rapid formation of classical invasive lobular carcinoma (CLC). Ninety mice of the Ecad;Pten mouse model (Boelens et al. 2016) are injected intraductally with lentiCre to induce Cre-conditional inactivation of E-cadherin and PTEN, resulting in formation of CLC. Animals are divided in five treatment groups: (i) daily oral vehicle (Medigel Sucralose, ClearH2O) and injection vehicle, (ii) daily A1 in oral vehicle, (iii) oral vehicle and twice weekly injection of anti-PD1 monoclonal antibody, (iv) A1 in oral vehicle and twice weekly injection of anti-PD1 monoclonal antibody and (v) oral vehicle and daily oral PI3K inhibitor BEZ-235. Throughout the experiment, mice are weighed every 48 h to evaluate tolerability and tumors are measured every 48 h. Treatment will start when tumor volume reaches 75 mm³ and lasts six weeks, after which animals are sacrificed. Tumors are weighed and tissues (including tumors and lymph nodes) are examined using histological and other techniques for comparing the tolerability and effects of the different treatments.

### Results

### Example 1—Anti-Invasive Flavonoids

The synthesis of the target flav(an)ones proceeded from appropriately substituted 2′-hydroxyacetophenones and benzaldehydes via intermediacy of 2′-hydroxychalcones. Due to the presence of the 2′-hydroxyl group, the Claisen-Schmidt condensation in the first step prompted use of excess benzaldehyde and base in 1,2-dimethoxyethane at 60° C.

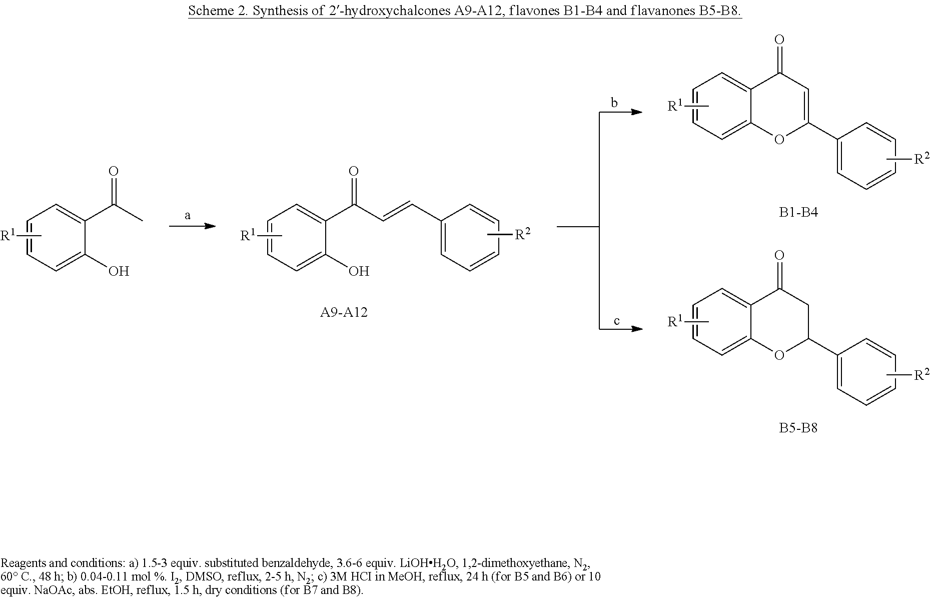

Mild oxidative cyclization of chalcones A9-A12 to flavones B1-B4 was accomplished in DMSO, using a catalytic amount of 12. This method proved fast and convenient and delivered the desired products in high yield and purity, often not requiring further purification via crystallization.

Treatment of A9-A10 with HCl in MeOH at reflux temperature afforded flavanones B5-B6 in moderate yields. The starting material was completely consumed after 24 h, but purification (extraction and crystallization in EtOH) led to considerable product loss and moderate yields (44-50%). (Hsieh et al. 1998) Treatment of chalcones A11-A12 with HCl in MeOH also resulted in equilibria with the desired flavanones B7-B8 after 18-24 h (approximately 70% conversion based on LC/MS data). We therefore also evaluated the cyclization of A11-A12 in basic environment (NaOAc in EtOH). Although this led to similar reaction equilibria (~70% conversion), work-up proved easier thus affording moderate yields of flavanones B7-B8.

The chick heart invasion (CHI) assay is a phenotypic assay that confronts precultured heart tissue fragments (PHFs, ‘normal tissue’), obtained from 9-day-old chicken embryos, with aggregates of human invasive MCF-7/6 mammary carcinoma cells (Bracke et al. 2014). In the control situation, the MCF-7/6 cells encircle, invade and destroy the PHF over an 8-day period. Invasion in this assay is defined as the progressive occupation and destruction of the PHF by the MCF-7/6 cells. Three-dimensional reconstruction of this interaction is obtained by microscopic analysis of all consecutive histological sections of the culture. The read-out is an invasion score according to a 5-grades histological scale.

The CHI closely reproduces the temporal, spatial and histological invasion patterns in man. The co-culture set-up probes for effects on both tumor cells and host tissue, and thus interrogates potential pharmacological targets in either interaction partner. Its use of histological evaluation allows discerning anti-invasive action from cytotoxicity or other effects.

We have submitted flavones B1-B4, flavanones B5-B8 and the intermediate 2′-hydroxychalcones A9, All and A12 to the chick heart invasion assay using MCF-7/6 aggregates. All compounds were evaluated at 1 μM and 100 nM; depending on the outcome higher or lower concentrations were subsequently evaluated. Table 1 displays the lowest concentration at which an anti-invasive effect was observed (histological grade I or II—cfr. Bracke et al., 2014). This table also includes CHI data on chalcones A1, A2 and A5. Table 2 displays the anti-invasive effect of A1 and B1 in the CHI assay using 4T1-Luc aggregates

The results indicate that all tested compounds show suitable activity, although particular substitution patterns lead to molecules with higher activity.

The anti-invasive activity data confirm that combinations of a (tri)methoxy decoration pattern at the left-hand side of the molecule with a halo (e.g., fluoro, chloro) substituent at the right-hand side can be favorable.

TABLE 1
Activity data for the compounds in the CHI assay with MCF-7/6 aggregates.

| Compound | Structure | Lowest active concentration (μM) |
|---|---|---|
| A1 | 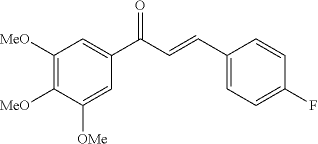 [SMILES: COc1cc(C(=O)/C=C/c2ccc(F)cc2)cc(OC)c1OC] | 0.01 |
| A10 | 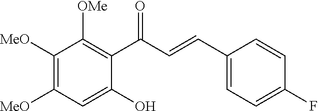 [SMILES: COc1cc(O)c(C(=O)/C=C/c2ccc(F)cc2)c(OC)c1OC] | 0.01 |
| B5 | 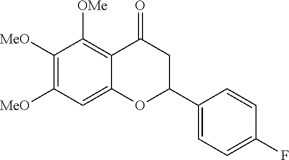 [SMILES: COc1cc2c(c(OC)c1OC)C(=O)CC(c1ccc(F)cc1)O2] | 0.1 |
| B1 | 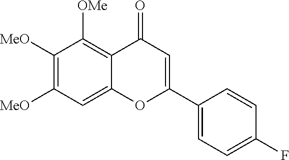 [SMILES: COc1cc2oc(-c3ccc(F)cc3)cc(=O)c2c(OC)c1OC] | 0.01 |
| B6 | 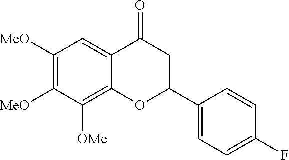 [SMILES: COc1cc2c(c(OC)c1OC)OC(c1ccc(F)cc1)CC2=O] | 100 |
| B2 | 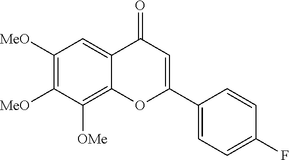 [SMILES: COc1cc2c(=O)cc(-c3ccc(F)cc3)oc2c(OC)c1OC] | 10 |
| A5 | 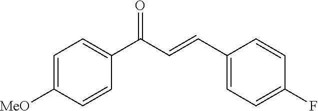 [SMILES: COc1ccc(C(=O)/C=C/c2ccc(F)cc2)cc1] | 1 |
| A12 | 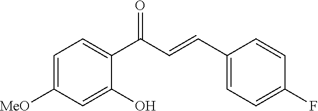 [SMILES: COc1ccc(C(=O)/C=C/c2ccc(F)cc2)c(O)c1] | 1 |
| B7 | 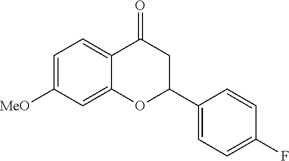 [SMILES: COc1ccc2c(c1)OC(c1ccc(F)cc1)CC2=O] | 10 |
| B3 | 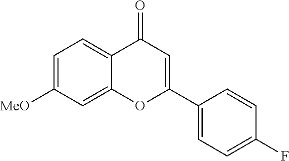 [SMILES: COc1ccc2c(=O)cc(-c3ccc(F)cc3)oc2c1] | 0.01 |
| A2 | 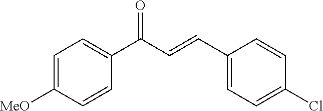 [SMILES: COc1ccc(C(=O)/C=C/c2ccc(Cl)cc2)cc1] | 0.01 |
| A13 | 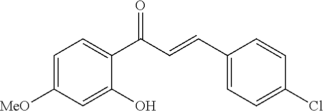 [SMILES: COc1ccc(C(=O)/C=C/c2ccc(Cl)cc2)c(O)c1] | 0.1 |
| B8 | 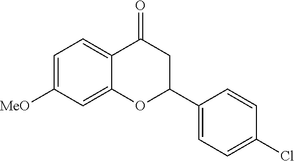 [SMILES: COc1ccc2c(c1)OC(c1ccc(Cl)cc1)CC2=O] | 0.1 |
| B4 | 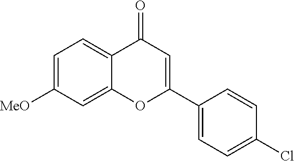 [SMILES: COc1ccc2c(=O)cc(-c3ccc(Cl)cc3)oc2c1] | 0.01 |

TABLE 2
Activity data for compounds A1 and B1 at 1 μM in the CHI assay with 4T1-Luc aggregates.

| Compound | Structure | Histological invasion grade |
|---|---|---|
| A1 | 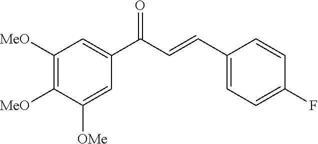 [SMILES: COc1cc(C(=O)/C=C/c2ccc(F)cc2)cc(OC)c1OC] | I/II-III |
| B1 | 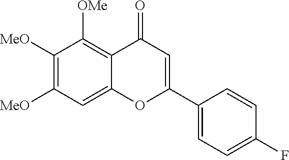 [SMILES: COc1cc2oc(-c3ccc(F)cc3)cc(=O)c2c(OC)c1OC] | II/II |

### Example 2—Modulation of the Tumor Microenvironment (TME)

### CAF Selectivity

13B2 hTERT breast CAFs showed a significant decrease in Young's modulus upon treatment with 1 μM A1 or B1, whilst 1 μM of the inactive control compound A8 did not have an effect. Compound A14 also decreased the Young's modulus of 13B2 hTERT breast CAFs at 1 μM. Using a concentration range of 30 nM-3 μM A1, a dose-response behavior was obtained (FIG. 1A-C). A similar result was obtained for CT5.3 hTERT colon CAF spheroids, as 1 μM A1 or B1 decreased the CT5.3 hTERT spheroid stiffness (FIG. 1D). A further similar result was obtained for glioblastoma CAF03 CAF spheroids, as 1 μM A1 decreased spheroid stiffness (FIG. 1E).

In an example of the CAF-selectivity of the fibroblast modulators in this invention, the reduction in spheroid stiffness due to 1 μM A1 or 1 μM B1 was observed in CAFs (FIG. 1A-E) but not in normal fibroblasts (HCA2 hTERT, FIG. 1F). A treatment of 0.03-3 μM A1 did, furthermore, not result in a change in spheroid stiffness for an epithelial cell line, such as MCF-7/6 (FIG. 1G), nor did it change the spheroid stiffness for primary glioblastoma cells (FIG. 1H). Notably, and in view of avoiding the risk of cardiac toxicity, treatment with 1 μM A1 or 1 μM B1 did not alter the spheroid stiffness of primary human atrial fibroblasts (FIG. 1I).

The novelty of this finding is exemplified by comparison of the selectivity of the fibroblast modulators in this invention with the behavior of existing CAF modulators myosin II-inhibitor blebbistatin, ROCK-inhibitor Y-27632 or actin polymerization inhibitor cytochalasin D (FIG. 2). While A1, B1 and said existing CAF-modulators were all active in spheroids of CAFs (13B2 hTERT, FIG. 2A; CAF03, FIG. 2B), A1 and B1 had no effect on the stiffness of spheroids of normal fibroblasts (HCA2 hTERT, FIG. 2C), spheroids of epithelial cells (MCF-7/6, FIG. 2D), spheroids of primary glioblastoma cells (FIG. 2E) or spheroids of primary human atrial fibroblasts (FIG. 2F). Said existing CAF modulators, however, did not only reduce the stiffness of spheroids of CAFs, but also reduced the stiffness of spheroids of normal fibroblasts (HCA2 hTERT), epithelial cells (MCF-7/6), primary glioblastoma cells and primary human atrial fibroblasts (FIG. 2A-F).

Selectivity data for further compounds of the invention for the reduction of the stiffness of spheroids of CAFs (13B2 hTERT) versus normal fibroblasts (HCA2 hTERT) is displayed in FIG. 3. The tested chalcones, flavones and flavanones are effective modulators of CAF spheroid stiffness but not of normal fibroblast stiffness. Combinations of one or more methoxy groups on one aromatic ring of the molecule and one or more halogens, with or without a hydroxyl group, on the other aromatic ring of the molecule, can be advantageous for the desired activity.

### Cell Viability

To investigate whether the effect observed in the AFM experiments was not caused by cytotoxicity, 2D and/or 3D CellTiter-Glo® Cell Viability Assay were performed on 13B2 hTERT, CT5.3 hTERT, HCA2 hTERT, MCF-7/6 and 4T1-Luc cells after treatment with A1 or B1. After 48 h of treatment, the metabolic activity of 2D cultures was inhibited in a dose-dependent manner with a high micromolar IC₅₀ value. Cells cultured as spheroids showed no response to treatment with A1 or B1 up to 100 μM. From these results, it can be concluded that the observed activity against CAFs is not due to a reduction of viability of these CAFs, nor that the observed selectivity for CAFs with respect to normal fibroblasts or cancer cells is due to a higher sensitivity of the CAFs in terms of viability reduction by A1 or B1.

### 4T1 Triple Negative Breast Cancer Model

The anti-metastatic efficacy of A1 and B1 was investigated in a 4T1-based intraductal model for triple-negative breast cancer (TBNC). A1 and B1 were added to Medigel for oral uptake and compared to DMSO added to Medigel as a negative control. Axillary lymph nodes, lung and liver metastases were the main parameters analyzed 6 weeks (w) post-inoculation (p.i.) of tumor cell suspensions in the mammary ducts, as well as histological analysis of the microenvironment of the primary tumor and axillary lymph nodes, lungs and liver.

### Tolerability

Body weight of all intraductally inoculated mice decreased during the first 3-4 days post-inoculation (p.i.) due to a decreased milk production after weaning of the pups (FIG. 4A), while body temperature remained stable during the first week p.i. in all mice (FIG. 4B). Upon weekly evaluation of body weight and temperature across the 6-week study period, the DMSO-, A1- and B1-treated mice showed no significant variation across the 6 w study period (FIG. 4A-B).

### Metastases

Treatment with A1 or B1 showed an overall reduction of the metastatic burden in lung, liver and axillary lymph node metastases compared to DMSO, based on ex-vivo bioluminescence data (FIG. 5A-C).

H&E images of the primary tumors corroborate the aggressive character of the 4T1-based intraductal model for triple negative breast cancer (TNBC) with tumor invasion in the mammary fat tissue and blood vasculature, areas of cellular necrosis, the presence of an immune infiltrate and tumor cells displaying a spindle shape characteristic for epithelial-to-mesenchymal transition (EMT) preceding metastasis. Smaller lung as well as liver metastases were found after A1 treatment, and smaller axillary lymph node metastases with B1 treatment compared to DMSO treatment (data not shown), corroborating the IVIS measurement-based bioluminescence data.

### Histology of TME of Primary Tumors and Metastases

Based on staining for α-smooth muscle actin (α-SMA), a CAF marker, the amount of CAFs in primary tumors significantly decreased by treatment with A1 or B1 compared to DMSO treatment (FIG. 6A).

Podoplanin (PDPN) as alternative marker for CAFs confirmed the α-SMA results, showing significantly decreased PDPN staining in primary tumors treated with A1 and B1 (P<0.001) (FIG. 6B). As an additional validation of both the α-SMA and PDPN staining, a significantly decreased staining was observed for CAF marker fibroblast activation protein alpha (FAP-α) in primary tumor tissue treated with A1 or B1 compared to DMSO (FIG. 6C). This serine protease is highly expressed in reactive fibroblasts and is indicated to regulate fibroblast growth.

Masson's trichrome staining, which visualizes collagen fibers, showed significantly decreased collagen deposition upon A1 or B1 treatment compared to DMSO (FIG. 6D) again corroborating the α-SMA, PDPN and also the FAP-α results for CAFs, and collectively identifying reduced tumor stiffness upon treatment with A1 and B1 compared to DMSO.

In addition to a decreased number of CAFs, CD11b staining of primary tumor tissue also showed significantly decreased amounts of myeloid cells upon treatment with A1 or B1 compared to DMSO (FIG. 6E). Ly6G staining, which is more specific for TANs and for neutrophil precursors such as granulocytic myeloid-derived suppressor cells (G-MDSCs), corroborated this reduction upon A1 or B1 treatment compared to DMSO (FIG. 6F). Complementing the innate immune cell modulation, CD163 staining of primary tumor tissue showed significantly decreased tumor-associated macrophages (TAMs) upon treatment with A1 or B1 (FIG. 6G). CD163 is a marker for M2-polarized macrophages, which promote tumor progression, angiogenesis, metastasis and immunosuppression. Due to these protumoral activities, associated with negative patient prognosis, the M2 macrophage phenotype is referred to as the tumor-associated macrophage (TAM). TAMs support an immunosuppressive TME through the secretion of cytokines, chemokines, growth factors. They also trigger the release of inhibitory immune checkpoint proteins in T cells.

In liver tissue, α-SMA and Masson's trichrome staining showed a significant reduction in CAFs and in collagen deposition at sites with proliferating myeloid cells upon A1 or B1 treatment compared to DMSO (FIGS. 7A and 7C). PDPN staining was also significantly decreased upon A1 or B1 treatment compared to DMSO in the liver tissue (FIG. 7B). Similar reductions were seen in axillary lymph node tissue and in lung tissue (FIG. 7D-G) (Liu et al. 2017)(Liu et al. 2017)(Liu et al. 2017).

### Cytokine Profile of the Primary Tumor

4T1 primary tumor lysates from animals treated with DMSO or A1 were used in a cytokine array that quantified the relative expression levels of 111 soluble mouse proteins. A total of 13 proteins were significantly affected by A1 treatment: CC-chemokine ligand (CCL) 6, CD40, chitinase-3-like protein 1 (CHI3L1), chemokine (C—X—C motif) ligand (CXCL) 16, fibroblast growth factor (FGF) acidic, interleukin (IL)-12 p40, low-density lipoprotein receptor (LDLR), MMP-3, MMP-9, myeloperoxidase (MPO), serine proteinase inhibitor (serpin) E1 and WNT1-inducible-signaling pathway protein 1 (WISP-1). This perturbation pattern points to an action not only selective for CAFs, but specifically for protumoral CAFs of the subtypes S1 and S4 (Costa et al., 2020). Both CAF-S₁ (CD29^{Med} FAP^{Hi} FSP1^{Med} aSMA^{Hi} PDGFRb^{Med-Hi} CAV1^{Low}) and CAF-S4 (CD29^{Hi} FAP^{Neg} FSP1^{Low}-Med aSMA^{Hi} PDGFRb^{Low-Med} CAV1^{Low}) subsets are detected in e.g. aggressive (HER2 and TN) breast cancer subtypes.

### REFERENCES

Wu J., Liang C., Chen M., Su W. Association between tumor-stroma ratio and prognosis in solid tumor patients: a systematic review and meta-analysis. Oncotarget. 2016; 7: 68954-68965
Kemi, N., Eskuri, M., Herva, A. et al. Tumour-stroma ratio and prognosis in gastric adenocarcinoma. Br J Cancer 119, 435-439 (2018).
Boelens, Mirjam C., Micha Nethe, Sjoerd Klarenbeek, Julian R. de Ruiter, Eva Schut, Nicola Bonzanni, Amber L. Zeeman, et al. 2016. “PTEN Loss in E-Cadherin-Deficient Mouse Mammary Epithelial Cells Rescues Apoptosis and Results in Development of Classical Invasive Lobular Carcinoma.” Cell Reports 16 (8): 2087-2101. https://doi.org/10.1016/j.celrep.2016.07.059.
Bracke, Marc E., Virinder S. Parmar, Anthony L. DePass, Christian V. Stevens, Barbara W. Vanhoecke, and Marc M. Mareel. 2014. Chick Heart Invasion Assay. Edited by Miriam Dwek, Udo Schumacher, and Susan A. Brooks. Methods in Molecular Biology, Vol. 1070. New York: Springer Science+Business Media. https://doi.org/10.1007/978-1-4614-8244-4_7.
Costa, A., Kieffer, Y., Scholer-Dahirel, A., Pelon, F., Bourachot, B., Cardon, M., Sirven, P., Magagna, I., Fuhrmann, L., Bernard, C., Bonneau, C., Kondratova, M., Kuperstein, I., Zinovyev, A., Givel, A. M., Parrini, M. C., Soumelis, V., Vincent-Salomon, A., & Mechta-Grigoriou, F. (2018). “Fibroblast Heterogeneity and Immunosuppressive Environment in Human Breast Cancer.” Cancer cell, 33(3), 463-479.e10. https://doi.org/10.1016/j.ccell.2018.01.011
Han, Chencheng, Tongyan Liu, and Rong Yin. 2020. “Biomarkers for Cancer-Associated Fibroblasts.” Biomarker Research 8 (1): 64. https://doi.org/10.1186/s40364-020-00245-w.
Hsieh, H K, T H Lee, J P Wang, J J Wang, and C N Lin. 1998. “Synthesis and Anti-Inflammatory Effect of Chalcones and Related Compounds.” Pharmaceutical Research 15 (1): 39-46. https://doi.org/10.1023/a:1011940401754.
Liu, Qiang, Hongfei Zhang, Xiaoli Jiang, Caiyun Qian, Zhuoqi Liu, and Daya Luo. 2017. “Factors Involved in Cancer Metastasis: A Better Understanding to ‘ Seed and Soil’ Hypothesis.” Molecular Cancer 16 (1): 176. https://doi.org/10.1186/s12943-017-0742-4.
Primac I, Maquoi E, Blacher S, Heljasvaara R, Van Deun J, Smeland H Y, Canale A, Louis T, Stuhr L, Sounni N E, Cataldo D, Pihlajaniemi T, Pequeux C, De Wever O, Gullberg D, Noel A. “Stromal integrin alpha11 regulates PDGFR beta signaling and promotes breast cancer progression.” J Clin Invest 2019; 129:4609-28. https://doi.org/10.1172/JC1125890.
Vlieghere, Elly De, F6lix Gremonprez, Laurine Verset, Lore Marien, Christopher J Jones, Bram De Craene, Geert Berx, et al. 2015. “Tumor-Environment Biomimetics Delay Peritoneal Metastasis Formation by Deceiving and Redirecting Disseminated Cancer Cells.” Biomaterials 54: 148-57. https://doi.org/10.1016/j.biomaterials.2015.03.012.

1. A compound of Formula I or a salt, hydrate, or solvate thereof, for use in the treatment of invasive lobular breast carcinoma, tumors having a Tumor-Stroma Ratio (TSR) of >50%; and/or pancreatic ductal adenocarcinoma; 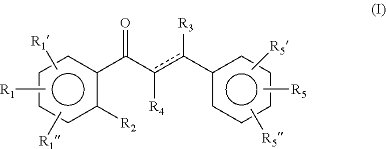
wherein [symbol]
represents a single or double bond;
each of said R₁, R₁′ and R₁″ is independently selected from the group consisting of —H, -halo, —CF₃, —OCF₃, and —OC₁₋₃alkyl;
R₂ is selected from the group consisting of —H, -halo, —CF₃, —OCF₃, and —OC₁₋₃alkyl;
R₃ is selected from the group consisting of —H, -halo, —C₁₋₃alkyl and —CF₃;
or R₂ and R₃ together with the C atoms to which they are attached form a 6-membered aromatic or non-aromatic heterocycle;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —S₀₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N; wherein each of said Het₁ is optionally substituted with from 1 to 3 substituents selected from the group consisting of —C₁₋₃alkyl, —OC₁₋₃alkyl, —C₁₋₃alkyl-OH and —OC₁₋₃alkyl-OH;
wherein at least one of said R₁, R₁′ and R₁″ is —OC₁₋₃alkyl or —CF₃;
wherein when R₂ and R₃ do not form a heterocycle then [symbol] represents a double bond.

2. The compound for use as defined in claim 1, wherein
when R₂ and R₃ form a 6-membered aromatic or non-aromatic heterocycle, then said compound is represented by formula (II) 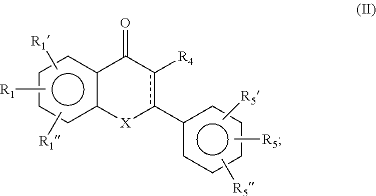
wherein X is selected from the group consisting of O, S, NH or N—C₁₋₃alkyl.

3. The compound for use as defined in anyone of claims 1 or 2, wherein [symbol]
represents a single or double bond;
each of said R₁, R₁′ and R₁″ is independently selected from the group consisting of —H, —CF₃ and —OC₁₋₂alkyl;
R₂ is —H;
R₃ is selected from the group consisting of —H, and -Me;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆, and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is a 6-membered heterocycle having from 1 to 3 heteroatoms selected from O and N; wherein each of said Het₁ is optionally substituted with from 1 to 3 substituents selected from the group consisting of —C₁₋₂alkyl, and —C₁₋₂alkyl-OH;
wherein at least one of said R₁, R₁′ and R₁″ is —OC₁₋₂alkyl; [symbol] wherein when R₂ and R₃ do not form a heterocycle then [symbol] represents a double bond.

4. The compound for use as defined in anyone of claims 1 to 3, wherein [symbol]
represents a single or double bond;
each of said R₁, R₁′ and R₁″ is independently selected from the group consisting of —H, —CF₃ and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —C₁₋₂alkyl, —NR₆R₇, —SO₂—NH₂, -Het₁ and -halo; each of said —C₁₋₂alkyl being optionally substituted with from 1 to 3 substituents selected from the group consisting of ═O, and -Het₁;
R₆, and R₇ are selected from the group consisting of —H, and —C₁₋₂alkyl; each of said —C₁₋₂alkyl being optionally substituted with from 1 to 3 substituents selected from the group consisting of ═O, and —NH-iPr,
Het₁ is selected from morpholinyl and piperazinyl; wherein each of said Het₁ is optionally substituted with from 1 to 3 substituents selected from the group consisting of —C₁₋₂alkyl, and —C₁₋₂alkyl-OH;
wherein at least one of said R₁, R₁′ and R₁″ is —OCH₃;
wherein when R₂ and R₃ do not form a heterocycle then [symbol] represents a double bond.

5. The compound for use as defined in anyone of claims 1 or 4, wherein [symbol]
represents a single or double bond;
each of said R₁, R₁′ and R₁″ is independently selected from the group consisting of —H, and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, and -halo;
wherein at least one of said R₁, R₁′ and R₁″ is —OCH₃;
wherein when R₂ and R₃ do not form a heterocycle then [symbol] represents a double bond.

6. The compound for use as defined in anyone of claims 1 to 5, wherein
each of said R₁, R₁′ and R₁″ is independently selected from the group consisting of —H—and —OCH₃;
R₂ is —H;
R₃ is —H;
or R₂ and R₃ together with the C atoms to which they are attached form a 6-membered aromatic or non-aromatic heterocycle,
R₄ is —H;
each of said R₅, R₅′ and R₅″ is independently selected from the group consisting of —H, —OH, —F and —Cl;
wherein at least one of said R₁, R₁′ and R₁″ is —OCH₃;
wherein when R₂ and R₃ do not form a heterocycle then [symbol] represents a double bond.

7. The compound for use as defined in any one of claims 1 to 5; wherein said compound is represented by anyone of formula (Ia), (IIa) or (IIb)

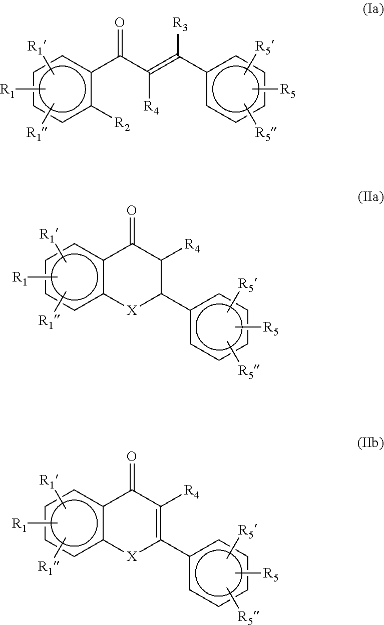

8. The compound for use as defined in anyone of claims 1 to 5, wherein said compound or a salt, hydrate or solvate thereof is selected from the list comprising:

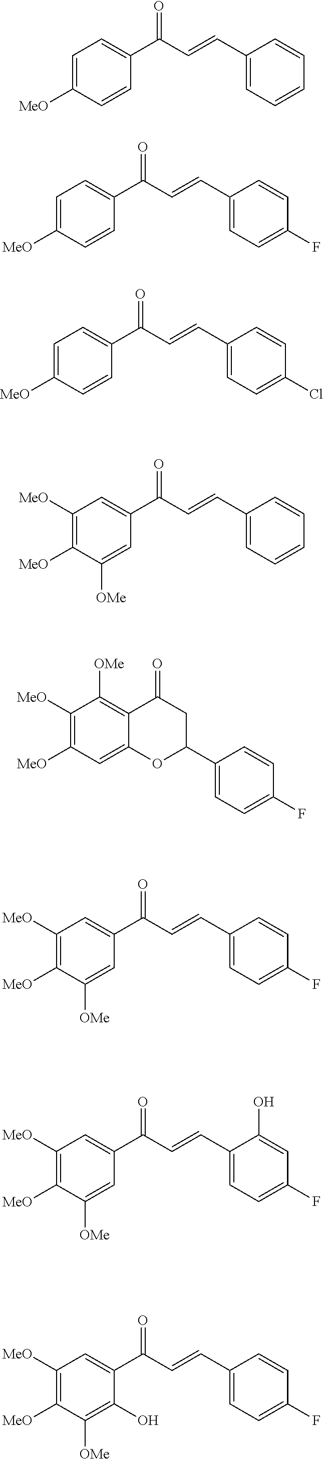

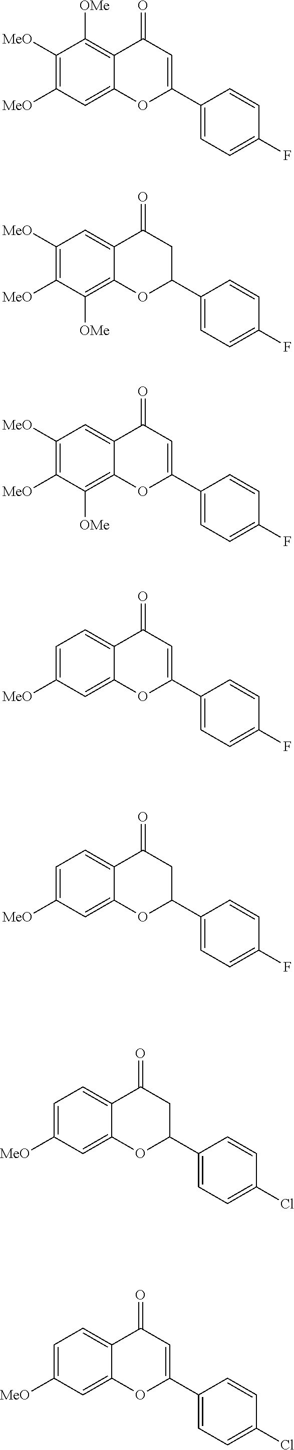

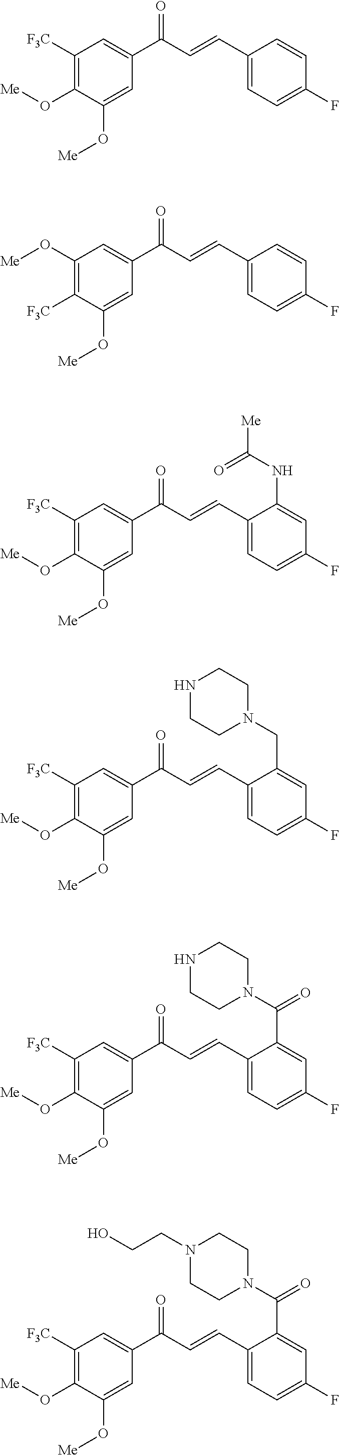 [SMILES: COc1cc(C(=O)/C=C/c2ccc(F)cc2)cc(C(F)(F)F)c1OC ; COc1cc(C(=O)/C=C/c2ccc(F)cc2)cc(OC)c1C(F)(F)F ; COc1cc(C(=O)/C=C/c2ccc(F)cc2NC(C)=O)cc(C(F)(F)F)c1OC ; COc1cc(C(=O)/C=C/c2ccc(F)cc2CN2CCNCC2)cc(C(F)(F)F)c1OC ; COc1cc(C(=O)/C=C/c2ccc(F)cc2C(=O)N2CCNCC2)cc(C(F)(F)F)c1OC ; COc1cc(C(=O)/C=C/c2ccc(F)cc2C(=O)N2CCN(CCO)CC2)cc(C(F)(F)F)c1OC]

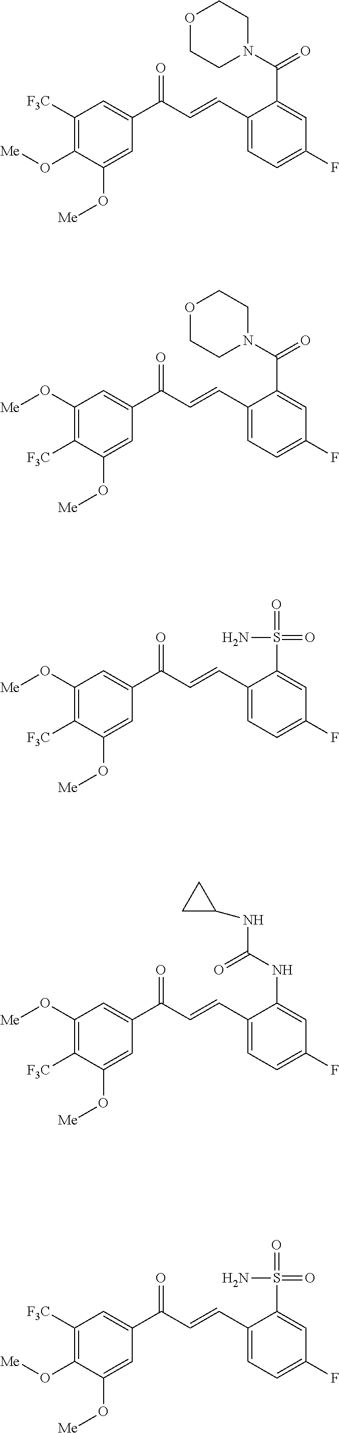 [SMILES: COc1cc(C(=O)/C=C/c2ccc(F)cc2C(=O)N2CCOCC2)cc(C(F)(F)F)c1OC ; COc1cc(C(=O)/C=C/c2ccc(F)cc2C(=O)N2CCOCC2)cc(OC)c1C(F)(F)F ; COc1cc(C(=O)/C=C/c2ccc(F)cc2S(N)(=O)=O)cc(OC)c1C(F)(F)F ; COc1cc(C(=O)/C=C/c2ccc(F)cc2NC(=O)NC2CC2)cc(OC)c1C(F)(F)F ; COc1cc(C(=O)/C=C/c2ccc(F)cc2S(N)(=O)=O)cc(C(F)(F)F)c1OC]

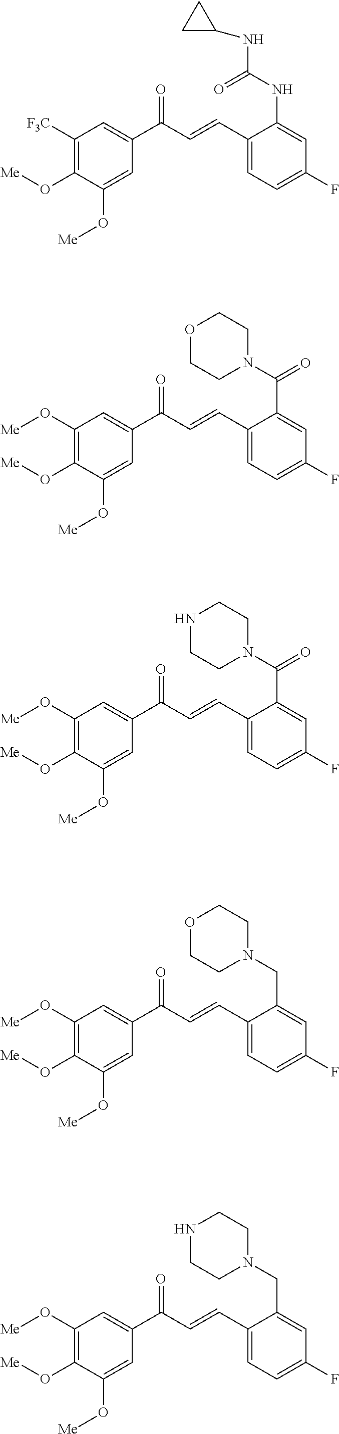 [SMILES: COc1cc(C(=O)/C=C/c2ccc(F)cc2NC(=O)NC2CC2)cc(C(F)(F)F)c1OC ; COc1cc(C(=O)/C=C/c2ccc(F)cc2C(=O)N2CCOCC2)cc(OC)c1OC ; COc1cc(C(=O)/C=C/c2ccc(F)cc2C(=O)N2CCNCC2)cc(OC)c1OC ; COc1cc(C(=O)/C=C/c2ccc(F)cc2CN2CCOCC2)cc(OC)c1OC ; COc1cc(C(=O)/C=C/c2ccc(F)cc2CN2CCNCC2)cc(OC)c1OC]

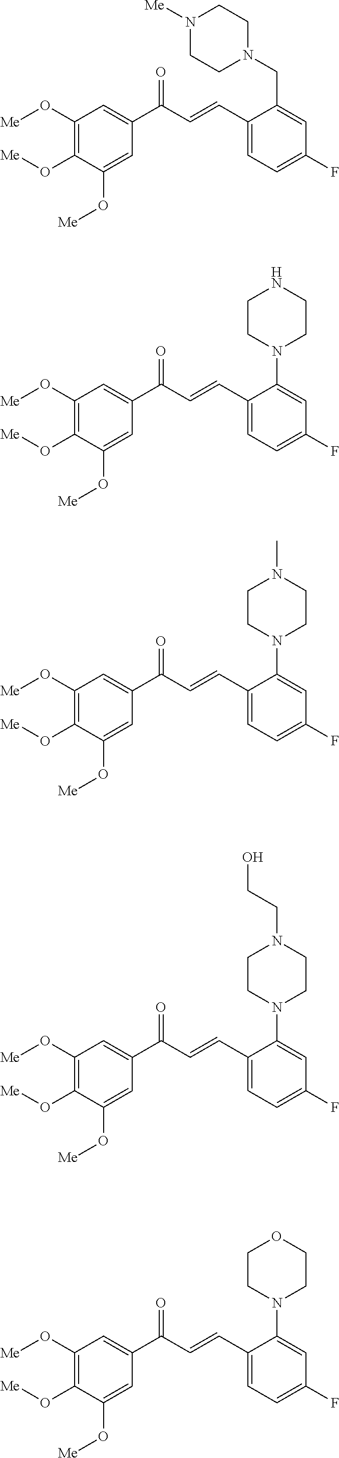 [SMILES: COc1cc(C(=O)/C=C/c2ccc(F)cc2CN2CCN(C)CC2)cc(OC)c1OC ; COc1cc(C(=O)/C=C/c2ccc(F)cc2N2CCNCC2)cc(OC)c1OC ; COc1cc(C(=O)/C=C/c2ccc(F)cc2N2CCN(C)CC2)cc(OC)c1OC ; COc1cc(C(=O)/C=C/c2ccc(F)cc2N2CCN(CCO)CC2)cc(OC)c1OC ; COc1cc(C(=O)/C=C/c2ccc(F)cc2N2CCOCC2)cc(OC)c1OC]

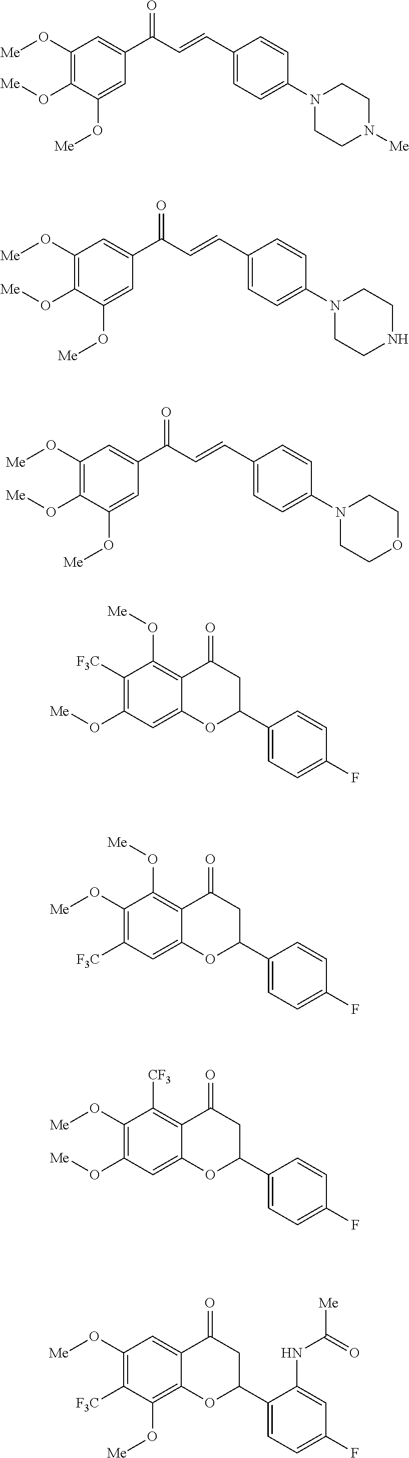

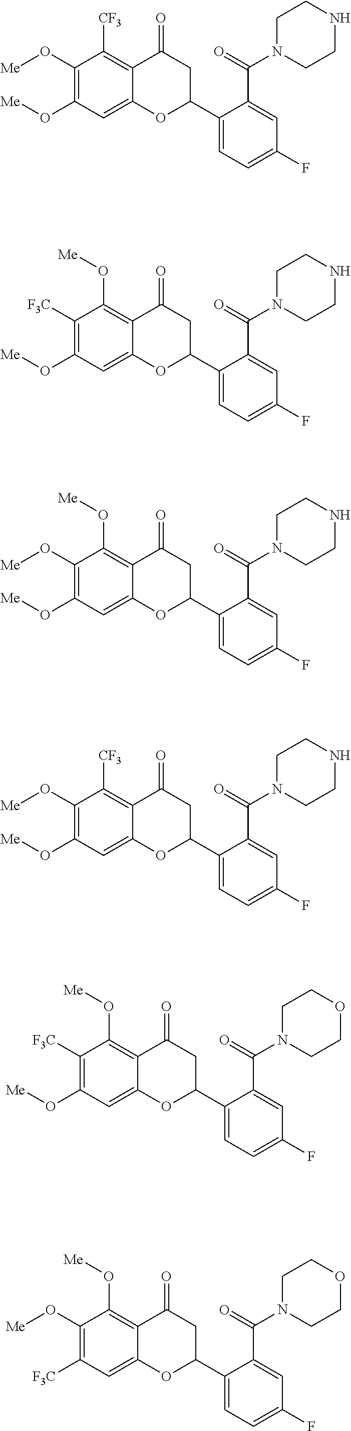 [SMILES: COc1cc2c(c(C(F)(F)F)c1OC)C(=O)CC(c1ccc(F)cc1C(=O)N1CCNCC1)O2 ; COc1cc2c(c(OC)c1C(F)(F)F)C(=O)CC(c1ccc(F)cc1C(=O)N1CCNCC1)O2 ; COc1cc2c(c(OC)c1OC)C(=O)CC(c1ccc(F)cc1C(=O)N1CCNCC1)O2 ; COc1cc2c(c(C(F)(F)F)c1OC)C(=O)CC(c1ccc(F)cc1C(=O)N1CCNCC1)O2 ; COc1cc2c(c(OC)c1C(F)(F)F)C(=O)CC(c1ccc(F)cc1C(=O)N1CCOCC1)O2 ; COc1c(C(F)(F)F)cc2c(c1OC)C(=O)CC(c1ccc(F)cc1C(=O)N1CCOCC1)O2]

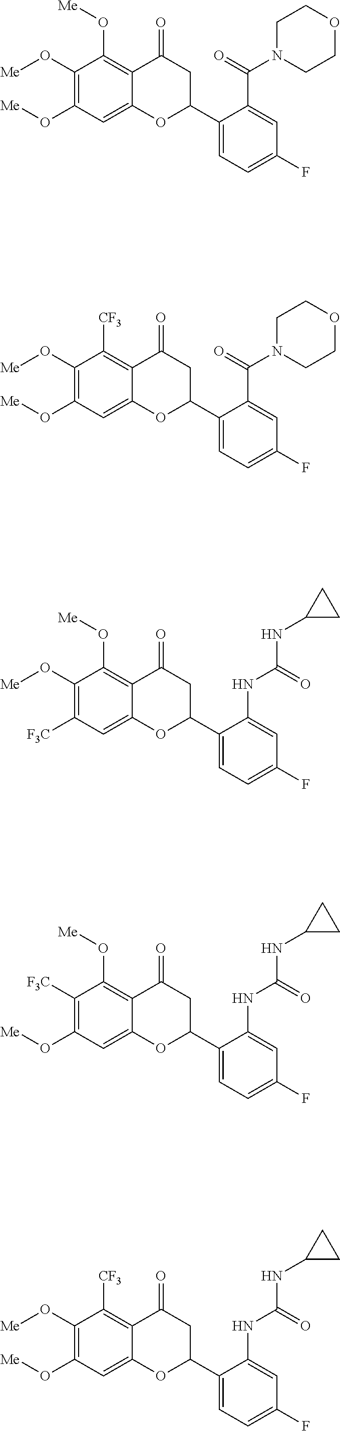 [SMILES: COc1cc2c(c(OC)c1OC)C(=O)CC(c1ccc(F)cc1C(=O)N1CCOCC1)O2 ; COc1cc2c(c(C(F)(F)F)c1OC)C(=O)CC(c1ccc(F)cc1C(=O)N1CCOCC1)O2 ; COc1c(C(F)(F)F)cc2c(c1OC)C(=O)CC(c1ccc(F)cc1NC(=O)NC1CC1)O2 ; COc1cc2c(c(OC)c1C(F)(F)F)C(=O)CC(c1ccc(F)cc1NC(=O)NC1CC1)O2 ; COc1cc2c(c(C(F)(F)F)c1OC)C(=O)CC(c1ccc(F)cc1NC(=O)NC1CC1)O2]

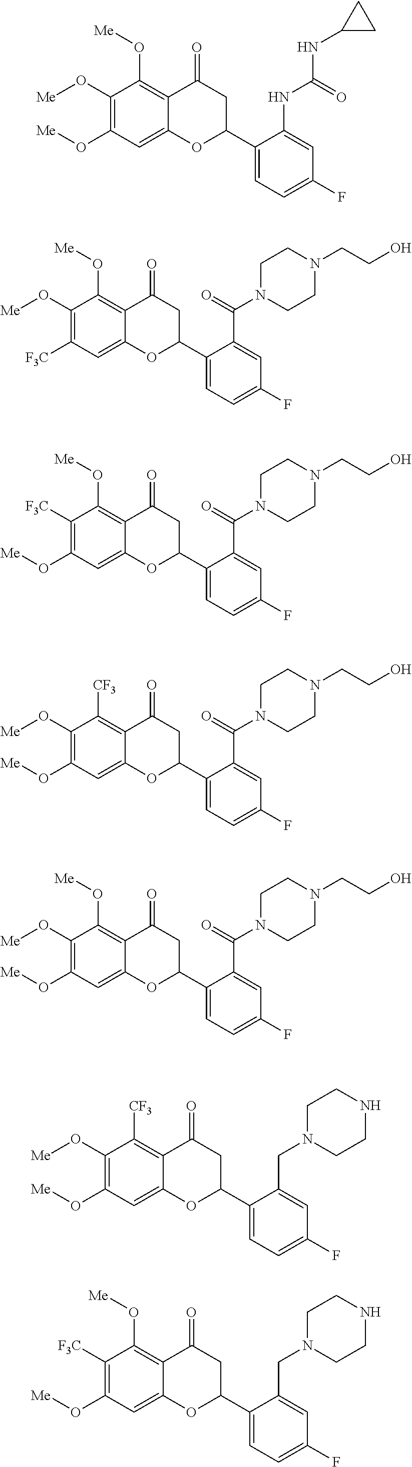

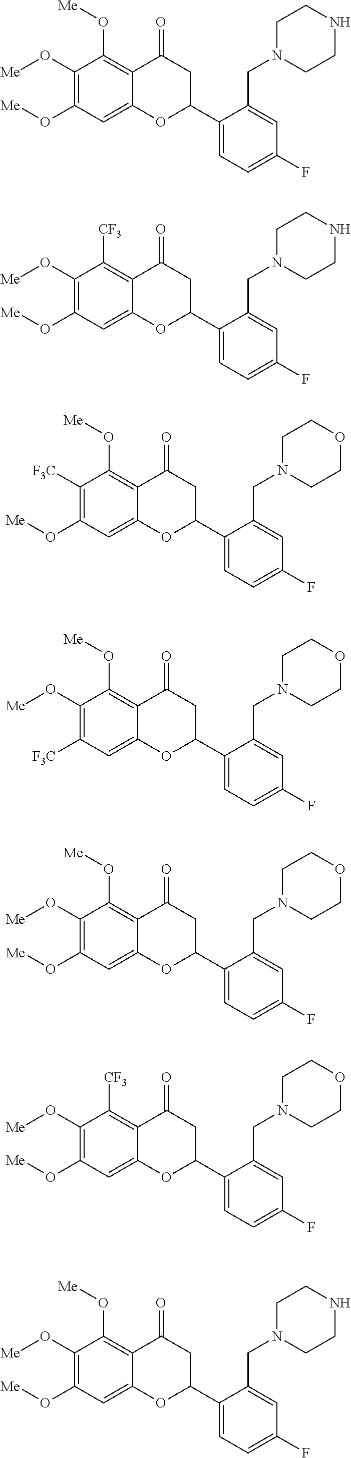

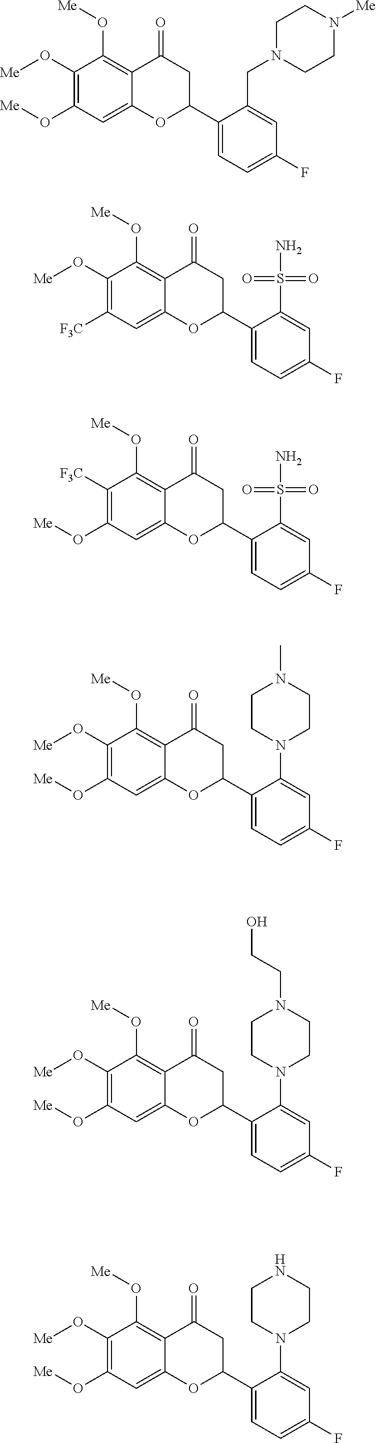 [SMILES: COc1cc2c(c(OC)c1OC)C(=O)CC(c1ccc(F)cc1CN1CCN(C)CC1)O2 ; COc1c(C(F)(F)F)cc2c(c1OC)C(=O)CC(c1ccc(F)cc1S(N)(=O)=O)O2 ; COc1cc2c(c(OC)c1C(F)(F)F)C(=O)CC(c1ccc(F)cc1S(N)(=O)=O)O2 ; COc1cc2c(c(OC)c1OC)C(=O)CC(c1ccc(F)cc1N1CCN(C)CC1)O2 ; COc1cc2c(c(OC)c1OC)C(=O)CC(c1ccc(F)cc1N1CCN(CCO)CC1)O2 ; COc1cc2c(c(OC)c1OC)C(=O)CC(c1ccc(F)cc1N1CCNCC1)O2]

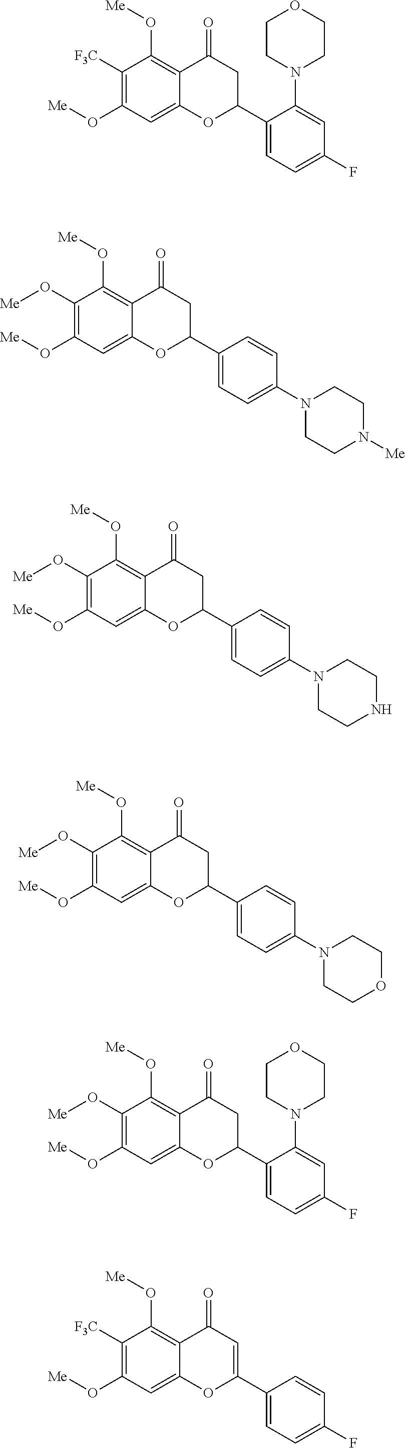 [SMILES: COc1cc2c(c(OC)c1C(F)(F)F)C(=O)CC(c1ccc(F)cc1N1CCOCC1)O2 ; COc1cc2c(c(OC)c1OC)C(=O)CC(c1ccc(N3CCN(C)CC3)cc1)O2 ; COc1cc2c(c(OC)c1OC)C(=O)CC(c1ccc(N3CCNCC3)cc1)O2 ; COc1cc2c(c(OC)c1OC)C(=O)CC(c1ccc(N3CCOCC3)cc1)O2 ; COc1cc2c(c(OC)c1OC)C(=O)CC(c1ccc(F)cc1N1CCOCC1)O2 ; COc1cc2oc(-c3ccc(F)cc3)cc(=O)c2c(OC)c1C(F)(F)F]

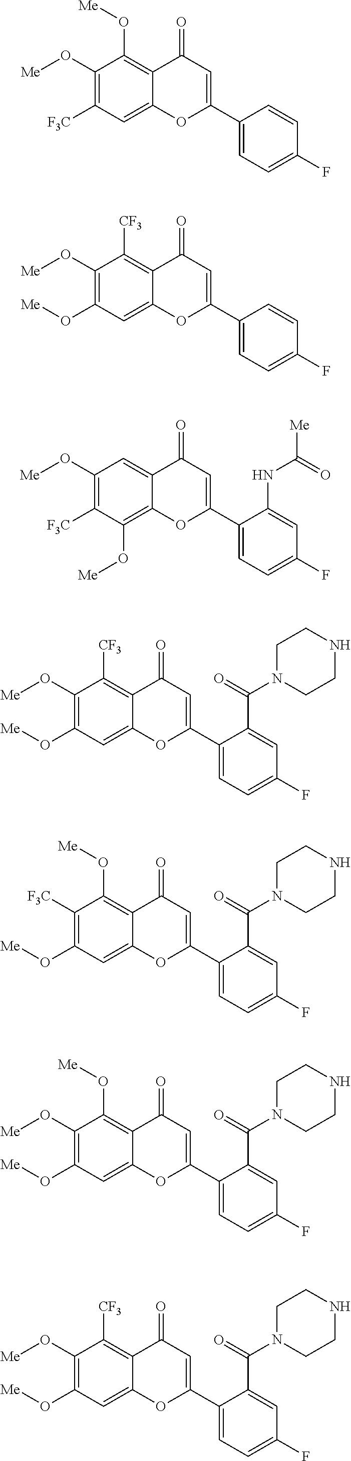

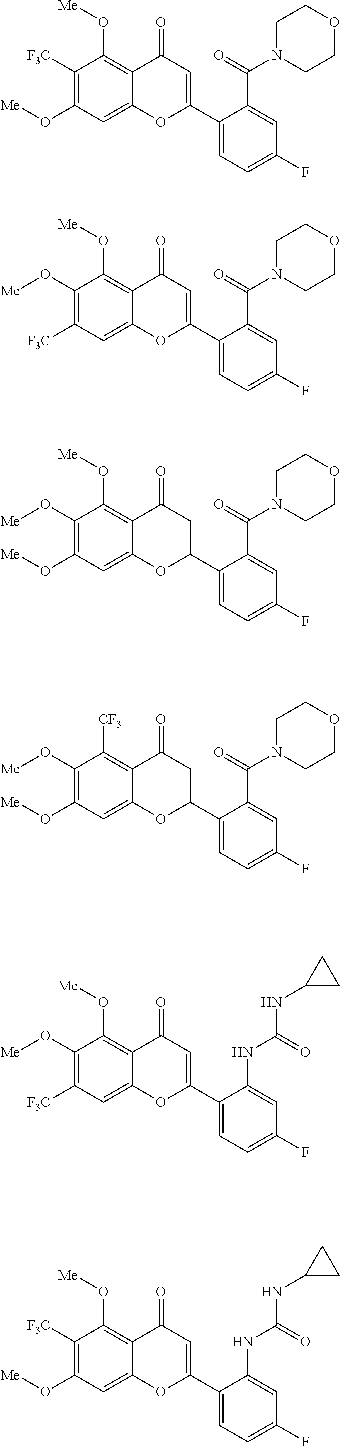 [SMILES: COc1cc2oc(-c3ccc(F)cc3C(=O)N3CCOCC3)cc(=O)c2c(OC)c1C(F)(F)F ; COc1c(C(F)(F)F)cc2oc(-c3ccc(F)cc3C(=O)N3CCOCC3)cc(=O)c2c1OC ; COc1cc2c(c(OC)c1OC)C(=O)CC(c1ccc(F)cc1C(=O)N1CCOCC1)O2 ; COc1cc2c(c(C(F)(F)F)c1OC)C(=O)CC(c1ccc(F)cc1C(=O)N1CCOCC1)O2 ; COc1c(C(F)(F)F)cc2oc(-c3ccc(F)cc3NC(=O)NC3CC3)cc(=O)c2c1OC ; COc1cc2oc(-c3ccc(F)cc3NC(=O)NC3CC3)cc(=O)c2c(OC)c1C(F)(F)F]

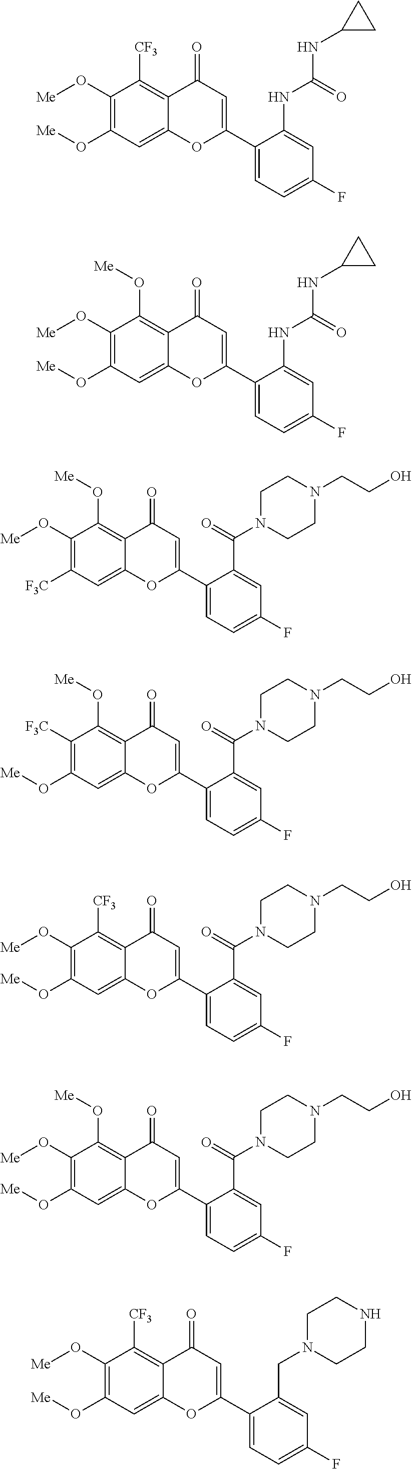

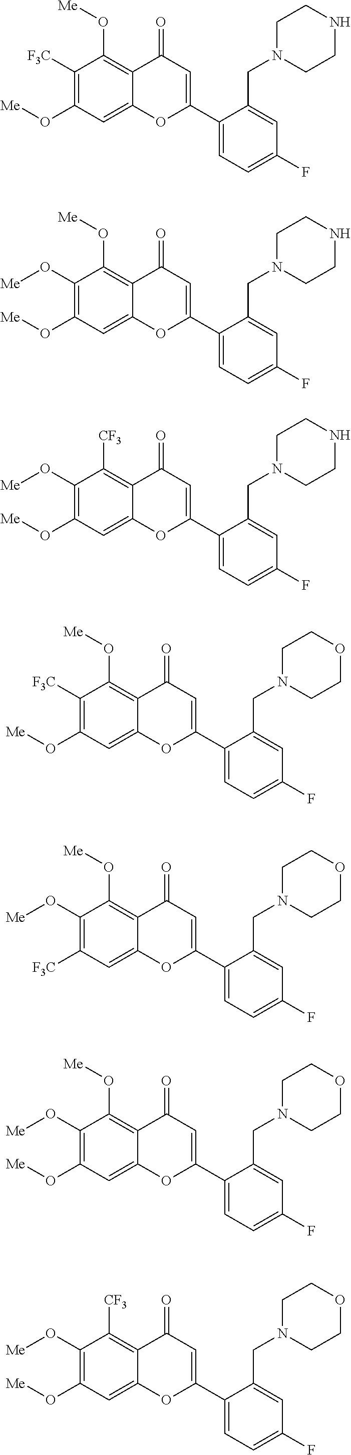

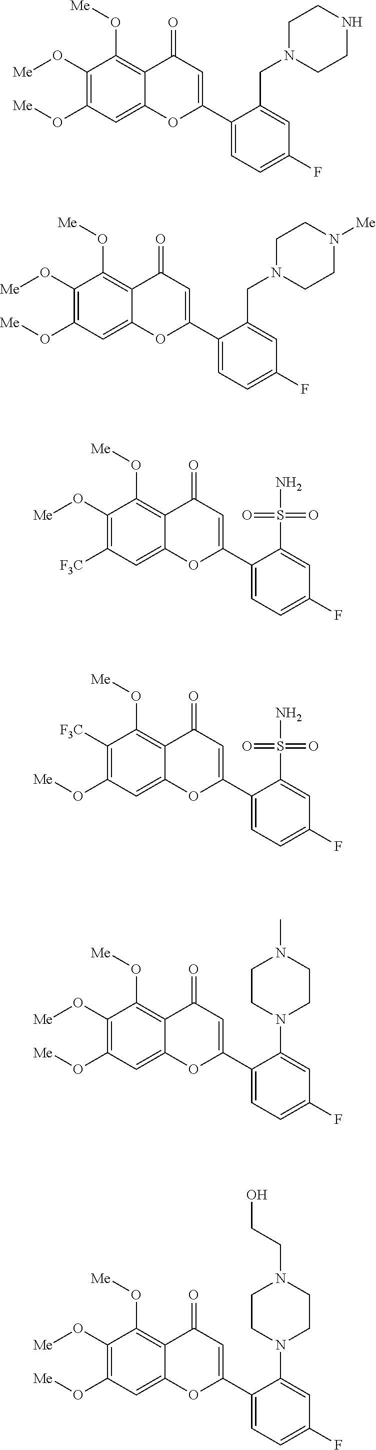 [SMILES: COc1cc2oc(-c3ccc(F)cc3CN3CCNCC3)cc(=O)c2c(OC)c1OC ; COc1cc2oc(-c3ccc(F)cc3CN3CCN(C)CC3)cc(=O)c2c(OC)c1OC ; COc1c(C(F)(F)F)cc2oc(-c3ccc(F)cc3S(N)(=O)=O)cc(=O)c2c1OC ; COc1cc2oc(-c3ccc(F)cc3S(N)(=O)=O)cc(=O)c2c(OC)c1C(F)(F)F ; COc1cc2oc(-c3ccc(F)cc3N3CCN(C)CC3)cc(=O)c2c(OC)c1OC ; COc1cc2oc(-c3ccc(F)cc3N3CCN(CCO)CC3)cc(=O)c2c(OC)c1OC]

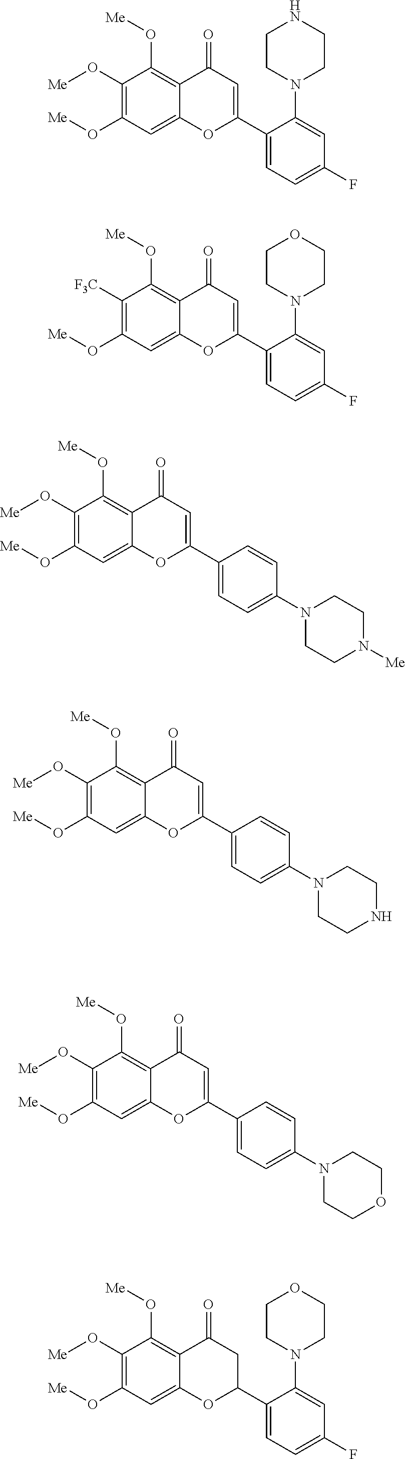 [SMILES: COc1cc2oc(-c3ccc(F)cc3N3CCNCC3)cc(=O)c2c(OC)c1OC ; COc1cc2oc(-c3ccc(F)cc3N3CCOCC3)cc(=O)c2c(OC)c1C(F)(F)F ; COc1cc2oc(-c3ccc(N4CCN(C)CC4)cc3)cc(=O)c2c(OC)c1OC ; COc1cc2oc(-c3ccc(N4CCNCC4)cc3)cc(=O)c2c(OC)c1OC ; COc1cc2oc(-c3ccc(N4CCOCC4)cc3)cc(=O)c2c(OC)c1OC ; COc1cc2c(c(OC)c1OC)C(=O)CC(c1ccc(F)cc1N1CCOCC1)O2]

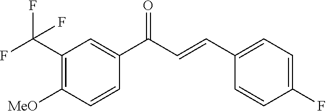 [SMILES: COc1ccc(C(=O)/C=C/c2ccc(F)cc2)cc1C(F)(F)F]

9. A compound or a salt, hydrate, or solvate thereof, selected from the list comprising:

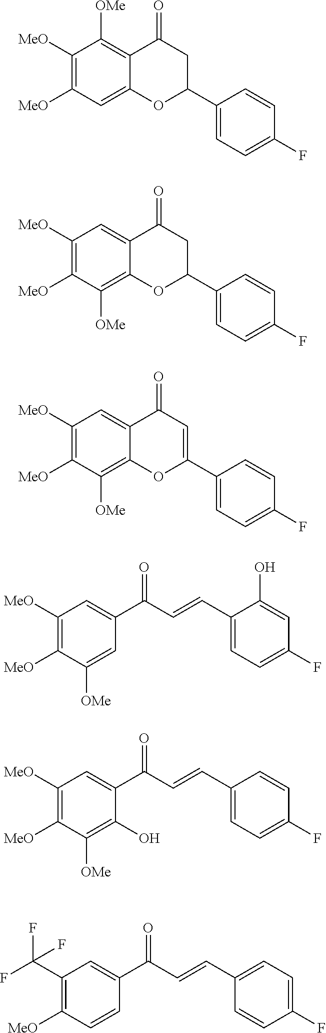

10. A pharmaceutical composition comprising a compound as defined in claim 9 and a pharmaceutically acceptable excipient.

11. A compound as defined in claim 9 or a composition as defined in claim 10; for use in human or veterinary medicine.

12. A compound as defined in claim 9 or a composition as defined in claim 10; for use in the treatment of cancer.

13. A compound as defined in claim 9, or a composition as defined in claim 10; for use in the treatment of invasive lobular breast carcinoma, tumors having a Tumor-Stroma Ratio (TSR) of >50%; and/or pancreatic ductal adenocarcinoma.

14. A compound for use as an anti-invasive compound of tumor cells in a subject having cancer; said compound being represented by any one of formula (IIa) or (IIb) 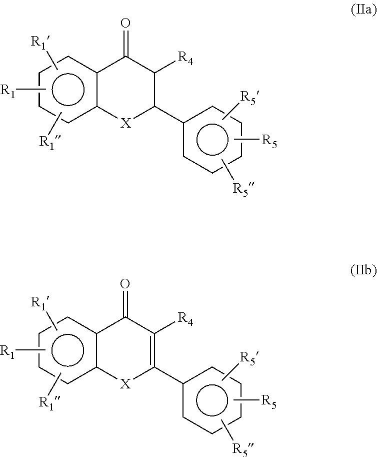
wherein
each of said R₁, R₁′ and R₁″ is independently selected from the group consisting of —H, -halo, —CF₃, —OCF₃, and —OC₁₋₃alkyl;
R₄ is selected from the group consisting of —H, -halo, —CF₃, —OCF₃, and —OC₁₋₃alkyl;
each of said R₅, R₅′ and R₅″ is independently selected from the group consisting of —H, —OH, —CN, —C₁₋₃alkyl, —OC₁₋₃alkyl, —NR₆R₇, —SO₂—NH₂, —SO₂-iPr, -Het₁ and -halo; each of said —C₁₋₃alkyl and —OC₁₋₃alkyl being optionally substituted with from 1 to 3 substituents selected from the group consisting of ═O, —NR₈R₉, and -Het₁;
R₆, R₇, R₈ and R₉ are selected from the group consisting of —H, and —C₁₋₃alkyl; each of said —C₁₋₃alkyl being optionally substituted with from 1 to 3 substituents selected from the group consisting of ═O, —NH₂, and —NH-iPr,
Het₁ is a 5- or 6-membered heterocycle having from 1 to 3 heteroatoms selected from S, O and N;
wherein each of said Het₁ is optionally substituted with from 1 to 3 substituents selected from the group consisting of —C₁₋₃alkyl, —OC₁₋₃alkyl, —C₁₋₃alkyl-OH and —OC₁₋₃alkyl-OH;
wherein at least one of said R₁, R₁′ and R₁″ is —OC₁₋₃alkyl or —CF₃.
